(19) United States
(12) Patent Application Publication
(10) Pub. No.: US 2014/0059718 A1
(43) Pub. Date: Feb. 27, 2014

(54) VECTORS FOR NUCLEIC ACID EXPRESSION IN PLANTS

(71) Applicant: Prisca Campanoni, Villars-Burquin (CH)

(72) Inventor: Prisca Campanoni, Villars-Burquin (CH)

(73) Assignee: PHILIP MORRIS PRODUCTS S.A., Neuchatel, ` (CH)

(21) Appl. No.: 13/980,123

(22) PCT Filed: Jan. 17, 2012
(86) PCT No.: PCT/EP2012/050630

§ 371 Date: Aug. 20, 2013

(30) Foreign Application Priority Data
Jan. 17, 2011 (EP) 11151187.9

(51) Int. Cl. C12N 15/82

(52) U.S. Cl. CPC C12N 15/8251
USPC 800/294; 435/320.1; 435/69.1; 435/419

(57) ABSTRACT

The present invention provides a binary vector containing only the elements essential for maintenance in E. coli and Agrobacterium and transforming plant cells, for single copy insertions in transgenic plants with little or no vector backbone integrations. The vectors of the invention are useful for stable or transient expression of one or more genes of interest.

The present invention is directed to vectors for expressing nucleic acids in plants and their applications. In particular, the vectors of the invention are useful for stable and transient expression of nucleic acids in a cell of a plant from the genus Nicotiana.

Overexpression, silencing and knock-out of a gene in a plant cell are powerful tools for studying gene expression and function in various plant tissues or subcellular locations. Regulated expression of exogenous nucleic acids in a plant cell is also useful for studying or engineering metabolic pathways with a view towards producing certain metabolites or proteins in selected plant organ(s) such as a root, leaf, stem, seed or trichome. Given the widespread interest in expressing nucleic acids in a plant cell, there is a need for vectors that are designed to be easy to use. It is an object of the present invention to meet these needs.

Various types of vectors have been constructed for the purpose of transforming a plant cell. Co-integrate vectors are hybrid tumour-inducing plasmids engineered for Agrobacterium-mediated transformation of plant cells and are constructed by homologous recombination of a bacterial plasmid with a transfer DNA (T-DNA) region of an Agrobacterium endogenous tumour-inducing plasmid (Zambryski et al., 1983, EMBO J. 2: 2143-2150). Binary vectors are vectors in which the virulence genes were placed on a different plasmid than the one carrying the T-DNA region (Bevan, 1984, Nucl. Acids. Res. 12: 8711-8721). The development of T-DNA binary vectors has made the transformation of plant cells easier as they do not require recombination.

The sizes of binary vectors for expression in plants are relatively large compared to bacterial plasmids and generally they have a lower copy number. Their size as well as their low copy number is a hurdle to cloning genes in such vectors, especially for high throughput screening. Multicopy binary vectors have been developed to facilitate the ease of cloning but these tend to result in multicopy T-DNA integrations in the plant nuclear genome and integration of binary vector backbone sequences. Multicopy T-DNA integrations are not desired as they tend to result in post-transcriptional gene silencing leading to low or no expression of the transferred nucleic acid and protein encoded by said nucleic acid. Integration of backbone vector DNA is also not desired from a regulatory perspective as the backbone is comprised of bacterial sequences with a function in bacteria. For Arabidopsis and tobacco, it was reported that up to 50% of the transgenic plants analysed contained vector backbone sequences, either linked to the left T-DNA border or right T-DNA border sequence. For Nicotiana tabacum W38, up to 75% of transgenic tobacco plants contained vector backbone sequences as established via PCR and Southern blot analysis. In these plants, the vector backbone sequences were linked either to T-DNA left or T-DNA right borders (Kononov et al., 1997. Plant J. 11: 945-957). WO 01/18192 discloses binary vectors for transformation of plants with Agrobacterium. pMRT1118 of 5970 bp, comprises a marker for selection in E. coli (nptIII); two origins of replication that are located on the plasmid adjacent to each other (a RK2 origin of replication and an on ColE1); gene for a replication initiator protein (trfA); Left and right borders of Agrobacterium tumefaciens (LB and RB); a selectable marker expressible in plants (nptII under promoter Pnos and terminator Tnos); and a multicloning site suitable for cloning further genes of interest.

Although many binary vectors have been developed for either stable or transient expression, they are only available for use in a limited number of plant species. There is a continuing need for improved binary vectors that can be used for various experimental and industrial purposes. When a vector is used to generate a transgenic plant, it is preferred that only a single copy is integrated without vector backbone sequence(s).

The present invention provides a binary vector containing basically only the elements which are essential for maintenance in E. coli and Agrobacterium and transforming plant cells, and results in a significant reduction of the size of the vector. The vectors of the invention are suitable for use in transient as well as stable transformation of plants and plant cells. Surprisingly, use of such vectors did not result in any vector backbone insertion at the right T-DNA border junction in single copy transgenic tobacco plants and only about 25% of the transgenic tobacco plants contained vector backbone sequences at the T-DNA left border junction. The vectors of the invention enabled the expression of one or more genes after Agrobacterium-mediated delivery to plants and cells of plants, such as Nicotiana tabacum and N. benthamiana.

Additional uses of the vectors include for example the screening for promoter activity or function of a cryptic nucleic acid sequence, for tissue specific expression including the direction of a gene expression product to a subcellular location. The vectors of the present invention can be used for stable as well as transient expression of a polypeptide of interest in cells of various plant species and are especially suitable for use in plants from the genus Nicotiana.

Accordingly, in a first embodiment of the present invention, a vector molecule is provided comprising, consisting of, or consisting essentially of the following nucleic acid elements:
a) a first nucleic acid element comprising a nucleotide sequence encoding a selectable marker which is functional in Escherichia coli and Agrobacterium species;
b) a second nucleic acid element comprising a nucleotide sequence of a first origin of replication which is functional in Escherichia coli;
c) a third nucleic acid element comprising a nucleotide sequence encoding a replication initiator protein;
d) a fourth nucleic acid element comprising a nucleotide sequence of a second origin of replication, which is different from the first origin of replication and which is functional in Agrobacterium; and
e) a fifth nucleic acid element comprising a nucleotide sequence of a T-DNA region comprising a T-DNA right border sequence and a T-DNA left border sequence of a tumour-inducing Agrobacterium tumefaciens plasmid or a root-inducing plasmid of Agrobacterium rhizogenes;
   wherein the above nucleic acid elements are provided on a circular polynucleotide molecule and are separated by gap nucleotide sequences which have no function in replication, maintenance or nucleic acid transfer, and wherein said gap nucleotide sequences account for less than 20%, 25%, 30%, 35%, 40%, 45%, of the total vector size. Preferably, the gap nucleotide sequences account for less than 20% of the total vector size.

In a specific embodiment of the invention, a vector molecule according to the present invention and as defined in any one of the preceding embodiments is provided, wherein
(i) the T-DNA left border sequence and the nucleotide sequence encoding a selectable marker (a) is separated by a first gap nucleotide sequence of not more than 300 bp;
(ii) the nucleotide sequence encoding a selectable marker (a) and the nucleotide sequence of a first origin of replication (b) is separated by a second gap nucleotide sequence of not more than 200 bp;
(iii) the nucleotide sequence of a first origin of replication (b) and the nucleotide sequence encoding a replication initiator protein (c) is separated by a third gap nucleotide sequence of not more than 200 bp;
(iv) the nucleotide sequence encoding a replication initiator protein (c) and the nucleotide sequence of a second origin of replication (d) is separated by a fourth gap nucleotide sequence of not more than 500 bp; and
(v) the nucleotide sequence of a second origin of replication (d) and the T-DNA right border sequence is separated by a fifth gap nucleotide sequence of not more than 150 bp.

In certain embodiments of the invention, the vector molecule according to the present invention and as defined in any one of the preceding embodiments has a total size of less than 5,900 bp, less than 5,500 bp, less than 5,200 bp, or less than 5,100 bp.

In a specific embodiment, the vector molecule according to the present invention and as defined in any one of the preceding embodiments has a total size of 5,150 bp.

In another specific embodiment of the invention, a vector molecule according to the present invention and as defined in any one of the preceding paragraph is provided, wherein the nucleic acid elements (a) through to (e) are arranged linearly relative to each other on the vector molecule in the order set out in the first embodiment of the invention, i.e, (a) (b) (c) (d) (e).

One skilled in the art will be readily capable of generating a vector molecule according to the invention and as defined in any one of the preceding embodiments comprising a backbone with a different order of the nucleic acids elements a) to e) as defined in any one of the preceding embodiments.

Accordingly, in one embodiment of the invention, the vector molecule according to the present invention and as defined in any one of the preceding embodiments is provided, wherein the nucleic acid element comprising a nucleotide sequence encoding a selectable marker functional in an Escherichia coli and Agrobacterium cell (a) is located proximally to the T-DNA left border sequence. In a specific embodiment, the nucleic acid element comprising a nucleotide sequence encoding a selectable marker functional in an Escherichia coli and Agrobacterium cell (a) and the T-DNA left border sequence is separated by a gap nucleotide sequence of not more than 300 bp.

In one embodiment of the invention, the vector molecule according to the present invention and as defined in any one of the preceding embodiments is provided, wherein the nucleic acid element comprising a nucleotide sequence encoding a selectable marker functional in an Escherichia coli and Agrobacterium cell (a) is located proximally to the T-DNA right border sequence. In a specific embodiment, the nucleic acid element comprising a nucleotide sequence encoding a selectable marker functional in an Escherichia coli and Agrobacterium cell (a) and the T-DNA right border sequence is separated by a gap nucleotide sequence of not more than 150 bp.

In one embodiment of the invention, the vector molecule according to the present invention and as defined in any one of the preceding embodiments is provided, wherein the nucleic acid elements comprising the nucleotide sequence of the first origin of replication (b) and the second origin of replication (d) are located proximally to the T-DNA left border sequence and the T-DNA right border sequence, respectively.

In a specific embodiment of the invention, the vector molecule according to the present invention and as defined in any one of the preceding embodiments is provided, wherein, the first origin of replication (b) and the second origin of replication (d) are not immediately adjacent to each other and at least one other functional element of the vector separates the first origin of replication (b) and the second origin of replication (d).

In another specific embodiment of the invention, the first origin of replication (b) and the second origin of replication (d) are selected from the group consisting of Col E1 on and RK2 oriV, respectively.

In one embodiment of the invention, the vector molecule according to the present invention and as defined in any one of the preceding embodiments is provided, wherein the nucleic acid element comprising the nucleotide sequence of the first origin of replication (b) is located proximally to the T-DNA left border sequence and the nucleic acid element comprising the nucleotide sequence of the second origin of replication (d) is located proximally to the T-DNA right border sequence.

In one embodiment of the invention, the vector molecule according to the present invention and as defined in any one of the preceding embodiments is provided, wherein the nucleic acid element comprising the nucleotide sequence of the first origin of replication (b) is located proximally to the T-DNA right border sequence and the nucleic acid element comprising the nucleotide sequence of the second origin of replication (d) is located proximally to the T-DNA left border sequence.

In one embodiment of the invention, the vector molecule according to the present invention and as defined in any one of the preceding embodiments is provided, wherein the first origin of replication (b) and the second origin of replication (d) are not immediately adjacent to each other and at least one other functional element of the vector separates the first origin of replication (b) and the second origin of replication (d).

In another embodiment, the nucleic acid element comprising the nucleotide sequence of a first origin of replication (b) or second origin of replication (d) and the T-DNA left border sequence is separated by a gap nucleotide sequence of not more than 300 bp. In still another embodiment, the nucleic acid element comprising the nucleotide sequence of a first origin of replication (b) or second origin of replication (d) and the T-DNA right border sequence is separated by a gap nucleotide sequence of not more than 150 bp.

In one embodiment of the invention, the vector molecule according to the present invention and as defined in any one of the preceding embodiments is provided, wherein the nucleic acid elements comprising the nucleotide sequences of the first origin of replication (b) and second origin of replication (d) are adjacent to each other and located proximally to the T-DNA left border sequence. In a specific embodiment, a vector molecule as defined in any one of the preceding embodiments is provided wherein the nucleic acid element comprising the nucleotide sequence of the first origin of replication (b) or the nucleotide sequence of the second origin of replication (d) and the T-DNA left border sequence is separated by a gap nucleotide sequence of not more than 300 bp and the nucleic acid elements comprising the nucleotide sequence of the first origin of replication (b) and the second origin of replication (d) are separated by a gap nucleotide sequence of not more than 200 bp.

In one embodiment of the invention, the vector molecule according to the present invention and as defined in any one of the preceding embodiments is provided, wherein the nucleic acid elements comprising the nucleotide sequences of the first origin of replication (b) and second origin of replication (d) are adjacent to each other and located proximally to the T-DNA right border sequence. In a specific embodiment of the invention, a vector molecule as defined in any one of the preceding embodiments is provided wherein the nucleic acid element comprising the nucleotide sequence of the first origin of replication (b) or the nucleotide sequence of the second origin of replication (d) and the T-DNA right border sequence is separated by a gap nucleotide sequence of not more than 150 bp and the nucleic acid elements comprising the nucleotide sequence of the first origin of replication (b) and the second origin of replication (d) are separated by a gap nucleotide sequence of not more than 500 bp.

In one embodiment of the invention, the vector molecule according to the present invention and as defined in any one of the preceding embodiments is provided, wherein the nucleic acid element comprising a nucleotide sequence encoding a replication initiator protein (c) is flanked by the nucleic acid elements comprising the nucleotide sequence of the first origin of replication (b) and the nucleotide sequence of the second origin of replication (d).

In one embodiment of the invention, the vector molecule according to the present invention and as defined in any one of the preceding embodiments is provided, wherein the nucleic acid element comprising a nucleotide sequence encoding a selectable marker functional in an Escherichia coli and Agrobacterium cell (a) is flanked by the nucleic acid elements comprising the nucleotide sequence of the first origin of replication (b) and the nucleotide sequence of the second origin of replication (d). In a specific embodiment, the flanking nucleic acid elements comprising the nucleotide sequence of the first origin of replication (b) and the nucleotide sequence of the second origin of replication (d) are separated from the nucleic acid elements comprising the nucleotide sequence encoding a replication initiator protein (c) or the nucleic acid elements comprising the nucleotide sequence encoding a selectable marker functional in an Escherichia coli and Agrobacterium cell (a) by a gap nucleotide sequence of not more than 200 bp and 500 bp, respectively.

In one embodiment of the invention, the vector molecule according to the present invention and as defined in any one of the preceding embodiments is provided, wherein the nucleic acid element (a) comprises a nucleotide sequence encoding a selectable marker functional in an Escherichia coli and Agrobacterium cell. The selectable marker may be an antibiotic resistance, particularly a resistance to an antibiotic selected from the group consisting of ampicillin, chloramphenicol, kanamycin, tetracycline, gentamycin, spectinomycin, bleomycin, phleomycin, rifampicin, streptomycin and blasticidin S.

In certain embodiments of the invention, the vector molecule according to the present invention and as defined in any one of the preceding embodiments is provided, wherein the nucleic acid element (b) comprises a nucleotide sequence of a first origin of replication functional in Escherichia coli selected from the group consisting of a ColE1 origin of replication, an origin of replication belonging to the ColE1 incompatibility group; a pMB1 origin of replication, and an origin of replication belonging to any one of the incompatibility group FI, FII, FIII, FIV, I J, N, O, P, Q, T, or W.

In a specific embodiment of the invention, the vector molecule according to the present invention and as defined in any one of the preceding embodiments is provided, wherein the nucleic acid element (b) comprises the nucleic acid of a ColE1 origin of replication. The ColE1 origin of replication can be obtained, for example, from a pBluescript vector (Agilent Technologies, Santa Clara, Calif., USA).

In another specific embodiment of the invention, the invention provides a vector molecule according to the present invention and as defined in any one of the preceding embodiments wherein the nucleic acid element (b) comprises the nucleic acid of a pMB1 origin of replication. The pMB1 origin of replication encodes two RNA's, RNAI and RNAII, and one protein known as Rom or Rop. For example, the pMB1 origin of replication can be that of a pGEM vector (Promega Corporation, Madison, Wis., USA) or a pUC vector such as, but not limited to, pUC8 (GenBank: L08959.1) and resulting in high copy number.

In one embodiment of the invention, the vector molecule according to the present invention and as defined in any one of the preceding embodiments is provided, wherein the nucleic acid element (c) comprises a nucleotide sequence encoding a replication initiator protein which is a RK2 TrfA replication initiator protein.

In certain embodiments of the invention, the vector molecule according to the present invention and as defined in any one of the preceding embodiments is provided, wherein the nucleic acid element (d) comprises a nucleotide sequence of a second origin of replication, which is different from the first origin of replication and is functional in Agrobacterium, and comprises a nucleotide sequence selected from the group consisting of a minimal oriV origin of replication, RK2 oriV, and an origin of replication belonging to any one of the incompatibility group FI, FII, FIII, FIV, I J, N, O, P, Q, T, or W.

In one embodiment of the invention, the vector molecule according to the present invention and as defined in any one of the preceding embodiments is provided, wherein the second nucleic acid element b) or the fourth nucleic acid element d) is the replication origin (oriV) and the third nucleic acid element c) is the TrfA replication initiator protein of the broad host range plasmid RK2, functional in both Escherichia coli and Agrobacterium spp. (Schmidhauser and Helinski (1985). J. Bacteriol. 164: 446-455).

In one embodiment of the invention, the vector molecule according to the present invention and as defined in any one of the preceding embodiments is provided, wherein the fifth nucleic acid element e) comprises two T-DNA border sequences, namely a T-DNA left border sequence and a T-DNA right border sequence.

In certain embodiments of the invention, the nucleic acid element e) comprises a T-DNA border sequence of an Agrobacterium spp. strain of the nopaline family, which is capable of catalyzing nopaline, nopalinic acid, leucinopine, glutaminopine or succinamopine.

In alternative embodiments of the invention, the nucleic acid element e) comprises a T-DNA border sequence of an Agrobacterium spp. strain of the octopine family, which is capable of catalyzing octopine, octopinic acid, lysopine or histopine. In certain other embodiments of the invention, the nucleic acid element e) comprises a T-DNA border sequence of an Agrobacterium spp. strain of the mannityl family catalyzing mannopine, mannopinic acid, agropine or agropinic acid.

In one embodiment of the invention, the vector molecule according to the present invention and as defined in any one of the preceding embodiments is provided, wherein the nucleic acid element (e) comprising a nucleotide sequence of a T-DNA region comprising a T-DNA right border sequence and a T-DNA left border sequence of an Agrobacterium tumefaciens tumour-inducing plasmid or an Agrobacterium rhizogenes root-inducing plasmid. contains at least one unique restriction endonuclease cleavage site, particularly at least two, three, four, or five unique restriction endonuclease cleavage sites.

The restriction endonuclease cleavage site may be a cleavage site selected form the group consisting of AatII, Acc65I, AcII, AfIII, AfIIII, AhdI, AloI, ApaBI, ApaI, AseI, AsiSI, AvrII, BaeI, BamHI, BanII, Bbr7I, BbsI, BbvCI, BfrBI, BlpI, BmtI, BplI, BpmI, Bpu10I, BsaAI, BsaI, BsaXI, BsiWI, BspEI, BsrGI, BstAPI, BstBI, BstZ17I, Bsu36I, DraIII, EcoICR1, EcoNI, EcoRI, FaII, FseI, FspAI, HindIII, HpaI, KpnI, M.AcII, M.AfIIII, M.AloI, M.ApaI, M.BaeI, M.BanII, M.BbvCIA, M.BbvCIB, M.BnaI, M.BsaAI, M.BstI, M.BstVI, M.DraIII, M.EcoAI, M.EcoKI, M.EcoR124I, M.HindIII, M.HpaI, M.KpnBI, M.KpnI, M.MunI, M.PaeR7I, M.PhiBssHII, M.PshAI, M.Rrh4273I, M.SacI, M.SalI, M.Sau3239I, M.SnaBI, M.Tth111I, M.VspI, M.XbaI, M.XhoI, MfeI, MluI, NheI, NruI, NsiI, PciI, PmII, Ppu10I, PshAI, PspOMI, PsrI, RsrII, SacI, SalI, SanDI, SapI, SciI, SnaBI, SrtI, SwaI, Tth111I, XbaI, XhoI, XmnI and ZraI. Such cleavage sites can accommodate the insertion of any DNA (such as an expression cassette) that comprises a compatible 5′ end, a compatible 3′ end, or one or two blunt ends.

In one embodiment, said expression cassette comprises a regulatory element that is functional in a plant, particularly a plant of the genus Nicotiana, and a nucleotide sequence of interest.

The skilled person in the art can readily remove an endonuclease recognition site that cuts once, or more, by mutating or altering one or more basepairs of the nucleic acid comprising said recognition site without altering the properties of the vector. It will be appreciated that any such restriction endonuclease recognition site that is outside of a coding sequence, regulatory sequence or other sequence with a function essential to the vector, can be altered without affecting the properties and function of the vector. Similarly, it will be appreciated that one can mutate a sequence comprised within a fragment coding for a protein without altering the function of said protein by introducing a silent mutation. It will be appreciated that one skilled in the art might not need an unique restriction site or any restriction site or combination of sites for cloning purposes since a nucleic acid sequence for expression in a plant cell, or any other nucleic acid sequence, can also be directly incorporated into the T-DNA region of the vector or elsewhere by design and chemically synthesized together with the nucleic acid elements a) to e) of the vector molecule according to the invention and as defined in any one of the preceding embodiments without the need to use restriction endonucleases.

The invention also provides a vector molecule as defined in any one of the preceding embodiments, wherein the fifth nucleic acid element (e) further comprises, between the T-DNA right border sequence and T-DNA left border sequence, a regulatory element which is functional in a transformed plant or plant cell and that will be operably linked to a nucleotide sequence encoding a protein of interest when such a nucleotide sequence is inserted in the vector molecule. Such vector molecules can be readily used for insertion of a nucleotide sequence of interest. The one or more unique restriction cleavage sites may be present between the regulatory element and one of the T-DNA border sequences to facilitate the insertion of a nucleotide sequence of interest. Accordingly, in certain embodiments the invention further provides a vector molecule as defined in any one of the preceding embodiments wherein the fifth nucleic acid element (e) further comprises, between the T-DNA right and T-DNA left border sequences, a regulatory element which is functional in a plant cell and which is operably linked to a nucleotide sequence encoding a protein of interest.

In various embodiments of the invention, the regulatory element that is present in the T-DNA region is a promoter selected from the group consisting of cauliflower mosaic virus 35S promoter, a modified cauliflower mosaic virus 35S promoter, a double cauliflower mosaic virus 35S promoter, a minimal 35 S promoter, nopaline synthase promoter, a cowpea mosaic virus promoter, a HT-CPMV promoter, a tobacco copalyl synthase CPS2p promoter, a dihydrinin promoter, a plastocyanin promoter, a 35S/HT-CPMV promoter, and many other promoters that are derived from caulimoviruses, such as but not limited to mirabilis mosaic virus (MMV), figwort mosaic virus (FMV), peanut chlorotic streak virus (PCLSV), double CaMV 35S promoter (35S×2), double MMV promoter (MMV×2), and double FMV promoter (FMV×2).

In certain embodiments of the invention, the nucleotide sequence under control of a plant regulatory element encodes a selectable marker which is functional in a plant cell, particularly a selectable marker selected from a group consisting of antibiotic resistance, herbicide resistance and a reporter protein or polypeptide that produces visually identifiable characteristics.

The plant selectable marker may be a marker providing resistance to an aminoglycoside antibiotic such as kanamycin or neomycin, a herbicide such as phosphinotricin or gluphosinate. In the alternative, the selectable marker may be a screenable marker such as a fluorescent protein including but not limited to green fluorescent protein (GFP).

However, for purpose of transient expression, the utility of a selectable marker for use in plant may be minimal and can be omitted from the vector. This allows a further significant reduction of the size of the vector. For example, as shown in example section 1.3, pPMP1 was constructed by deleting the pBIN61-derived neomycin phosphotransferase gene (nptII) encoding kanamycin resistance from pC100. Thus, pPMP1 is an example of a vector of the invention that lacks a plant selectable marker.

Accordingly, in one embodiment of the invention, the vector molecule according to the present invention and as defined in any one of the preceding embodiments is provided, wherein the plant selectable marker gene has been omitted.

In various embodiments of the invention, the nucleotide sequence encoding a protein of interest for expression in a plant or plant cell encodes, without limitation, an antigen, an immunogen, an active ingredient of a vaccine, a cytokine, a chemokine, a blood protein, a hormone, an enzyme, a growth factor, an antibody or a fragment thereof, and a suppressor of gene silencing.

The protein or polypeptide of interest may be an antigen or immunogen, isolated or derived from a respiratory syncytial virus (RSV), a rabies virus, an influenza virus, a Hepatitis virus or a Norwalk virus.

In a specific embodiment, the virus-like particle is composed of influenza haemagglutinin 5 (H5) which was successfully produced in a Nicotiana tabacum plant cell using the minimal vector-derived pC229 vector as described in Example 4. The influenza virus can be isolated from humans, domestic animals (e.g., swine, chicken, duck) or wild animals (e.g., migrating birds).

The protein or polypeptide may also be an enzyme such as a glucocerebrosidase, a glycosyltransferase, an esterase, or a hydrolase.

In certain embodiments, the vector molecule according to the present invention and as defined in any one of the preceding embodiments is provided, wherein the vector molecule further comprises in the T-DNA region a nucleotide sequence encoding a signal peptide that targets the newly expressed protein of interest to a subcellular location. Signal peptides that may be used within the vector molecules according the invention are, for example, those selected from a group consisting of a vacuolar targeting sequence, a chloroplast targeting sequence, a mitochondrial targeting sequence, a sequence that induces the formation of protein bodies in a plant cell or a sequence that induces the formation of oil bodies in a plant cell.

In one embodiment of the invention, the targeting sequence is a signal peptide for import of a protein into the endoplasmic reticulum. Signal peptides are transit peptides that are located at the extreme N-terminus of a protein and cleaved co-translationally during translocation across the endoplasmatic reticulum membrane. A signal peptide that can be used in a vector molecule according to the invention, without being limited thereto, is that naturally occurring at the N-terminus of a light or heavy chain sequence of an IgG, or the patatin signal peptide of pC148 as described in Example 3. Further signal peptides can, for example, be predicted by the SignalP prediction tool (Emanuelsson et al., 2007, Nature Protocols 2: 953-971).

In another embodiment of the invention, the targeting sequence may be an endoplasmatic reticulum retention peptide. Endoplasmatic reticulum retention targeting sequences occur at the extreme C-terminus of a protein and can be a four amino acid sequence such as KDEL, HDEL or DDEL, wherein K is lysine, D is aspartic acid, E is glutamic acid, L is leucine and H is histidine.

In still another embodiment of the invention, the targeting sequence may be a sequence that when fused to a protein results in the formation of non-secretory storage organelles in the endoplasmatic reticulum such as but not limited to those described in WO07/096,192, WO06/056483 and WO06/056484.

In certain embodiments of the invention, the targeting sequence can be a vacuolar targeting sequence, a chloroplast targeting sequence, a mitochondrial targeting sequence or any other sequence the addition of which results in a specific targeting of the protein fused there onto to a specific organelle within the plant or plant cell.

In one embodiment, the vector molecule according to the invention and as defined in any one of the preceding embodiments further comprises in the T-DNA region a site-specific recombination site for site-specific recombination.

In one embodiment, the site-specific recombination site is located downstream of the plant regulatory element. In another embodiment, the site-specific recombination site is located upstream of the plant regulatory element.

In a specific embodiment of the invention, the recombination site is a LoxP site and part of a Cre-Lox site-specific recombination system.

The Cre-Lox site-specific recombination system uses a cyclic recombinase (Cre) which catalyses the recombination between specific sites (LoxP) that contain specific binding sites for Cre.

In another specific embodiment, the recombination site is a Gateway destination site. For example, nucleic acids of interest are first cloned into a commercially available “entry vector” and subsequently recombined into a “destination vector”. The destination vector can be used for the analysis of promoter activity of a given nucleic acid sequence or number of sequences, for analysis of function, for protein localization, for protein-protein interaction, for silencing of a given gene or for affinity purification experiments.

In one embodiment of the invention, the vector molecule according to the present invention and as defined in any one of the preceding embodiments is provided, wherein the vector comprises in the T-DNA region a plant selectable marker gene that is under control of a regulatory element functional in a plant cell and a recombination site for site-specific recombination which is located between the T-DNA right border sequence and the plant selectable marker gene.

In another embodiment of the present invention, the vector molecule as defined in any one of the preceding embodiments further comprises a nucleotide sequence that encodes a suppressor of gene silencing.

In certain embodiments of the invention, the suppressor of gene silencing is of viral origin, particularly selected from the group consisting of Havel river virus (HaRV), pear latent virus (PeLV), lisianthus necrosis virus, grapevine Algerian latent virus, Pelargonium necrotic spot virus (PeNSV), Cymbidium ringspot virus (CymRSV), artichoke mottled crinkle virus (AMCV), carnation Italian ringspot virus (CIRV), lettuce necrotic stunt virus, rice yellow mottle virus (RYMV), potato virus X (PVX), African cassaya mosaic virus (ACMV), cucumber mosaic virus (CMV), cucumber necrosis virus (CNV), potato virus Y (PVY), or tomato bushy stunt virus (TBSV).

In certain embodiments of the invention, the suppressor of gene silencing is the helper-component proteinase (HcPro) of tobacco etch virus (TEV), the p1 protein of rice yellow mottle virus (RYMV), the p25 protein of potato virus X (PVX), the AC2 protein of African cassaya mosaic virus (ACMV), the 2b protein of cucumber mosaic virus (CMV), the 19 kDa p19 protein of cucumber necrosis virus (CNV), the p19 protein of tomato bushy stunt virus (TBSV), or the helper-component proteinase (HcPro) of potato virus Y (PVY), or tomato bushy stunt virus (TBSV).

In a specific embodiment of the invention, the suppressor of gene silencing is HcPro of tobacco etch virus (TEV) as disclosed by Mallory et al. (2001. Plant Cell 13: 571-583) and examplified in Example 4 for the production of an influenza H5 virus-like particle, or the p19 protein of Tomato bushy stunt virus (TBSV) as successfully used in Example 3 for the production of a rituximab monoclonal antibody in tobacco.

In one embodiment, the present invention relates to a vector molecule having a polynucleotide sequence being at least 80%, 81%, 82%, 83%, 84%, 85%, 86%, 87%, 88%, 89%, 90%, 91%, 92%, 93%, 94%, 95%, 96%, 97%, 98% or 99% identical to the polynucleotide sequence as depicted in SEQ ID NO: 1 and wherein the nucleic acid elements (a) to (e) exhibit the same functionality as the counterpart elements provided in SEQ ID NO:1.

In a specific embodiment, the vector molecule has a polynucleotide sequence as depicted in SEQ ID NO: 1.

The vectors of the present invention and the nucleic acid elements a) to e) as defined in any one of the preceding embodiments and comprised within such vectors may either be naturally occurring nucleic acid sequences covalently linked on a circular DNA plasmid, or chemically synthesized nucleic acid sequences, or a mixture thereof. When chemically synthesized, the nucleic acid elements a) to e) can be based on naturally occurring nucleic acid and protein or polypeptide sequences of bacteria or other organisms of interest, and exhibit the same functionality as the naturally occurring sequences.

The invention also encompasses bacterial cells, particularly a bacterial cell selected from the group of Rhizobium, Sinorhizobium, Mesorhizobiu, Bradyrhizobium, Pseudomonas, Azospirillum, Rhodococcus, Phyllobacterium, Xanthomonas, Burkholderia, Erwinia, Bacillus, Escherichia, and Agrobacterium, that comprises the vector molecule according to the invention and as defined in any one of the preceding embodiments.

In a specific embodiment, the invention relates to an Agrobacterium cell, particularly an Agrobacterium tumefaciens or an Agrobacterium rhizogenes cell, comprising the vector molecule according to the invention and as defined in any one of the preceding embodiments.

In another specific embodiment, the invention relates to a cell of an Agrobacterium tumefaciens strain selected from the group consisting of Agrobacterium strain AGL1, EHA105, GV2260, GV3101 and Chry5, but particularly Agrobacterium tumefaciens strain AGL1 or EHA105, comprising the vector molecule according to the invention and as defined in any one of the preceding embodiments.

The invention also encompasses a plant or plant cells that comprise a vector of the invention and as defined in any one of the preceding embodiments.

In a preferred embodiment, the plant or plant cell according to the invention and as defined in any one of the preceding embodiments, particularly a Nicotiana tabacum plant or plant cell which has been transformed by a vector molecule of the invention, contains a single copy of a T-DNA region or a functional part thereof which is integrated into the plant genome without the vector sequence that is adjacent to the left T-DNA border or the vector sequence that is adjacent to the right T-DNA border, or both.

The plant can be a monocotyledonous or a dicotyledonous plant including but not limited to those of the genus Nicotiana. Exemplary species of the Nicotiana genus include, but are not limited to: Nicotiana africana, Nicotiana amplexicaulis, Nicotiana arentsii, Nicotiana benthamiana, Nicotiana bigelovii, Nicotiana corymbosa, Nicotiana debneyi, Nicotiana excelsior, Nicotiana exigua, Nicotiana glutinosa, Nicotiana goodspeedii, Nicotiana gossei, Nicotiana hesperis, Nicotiana ingulba, Nicotiana knightiana, Nicotiana maritime, Nicotiana megalosiphon, Nicotiana miersii, Nicotiana nesophila, Nicotiana noctiflora, Nicotiana nudicaulis, Nicotiana otophora, Nicotiana palmeri, Nicotiana paniculata, Nicotiana petunioides, Nicotiana plumbaginifolia, Nicotiana repanda, Nicotiana rosulata, Nicotiana rotundifolia, Nicotiana rustica, Nicotiana setchelli, Nicotiana stocktonii, Nicotiana eastii, Nicotiana suaveolens or Nicotiana trigonophylla. Desirably the first tobacco plant is Nicotiana amplexicaulis, Nicotiana benthamiana, Nicotiana bigelovii, Nicotiana debneyi, Nicotiana excelsior, Nicotiana glutinosa, Nicotiana goodspeedii, Nicotiana gossei, Nicotiana hesperis, Nicotiana knightiana, Nicotiana maritima, Nicotiana megalosiphon, Nicotiana nudicaulis, Nicotiana paniculata, Nicotiana plumbaginifolia, Nicotiana repanda, Nicotiana rustica, Nicotiana suaveolens or Nicotiana trigonophylla.

In a specific embodiment, the invention relates to a plant or plant cells that comprise a vector of the invention and as defined in any one of the preceding embodiments, wherein said plant or plant cell is a Nicotiana tabacum plant. Exemplary varieties of Nicotiana tabacum include commercial varieties such as DAC Mata Fina, 81V9, Ottawa 705, Labu, TI 115, Havana 307, Xanthi, T190, Kentucky 16, Havana 38, Wisconsin 38, Con. Havana 38, Burley 49, 81V9 MS, Judy's Pride, CT 572, TI 158, Cannelle, T194, CT 157, White Mammoth, Kelly, Gold Dollar, White Gold, Bonanza, Havana 425, Delfield, Coker 48, Dehli 76, Yellow Mammoth, Burley 1, Delgold, Green Briar, TI 161, Maryland 201, Duquesne, CT 681, TI 170, TI 164, Kentucky 10, Bell C, TI 75, Vinica, Grande Rouge, Belgique 3007, 164, TI 124, TI 95, PO2, BY-64, AS44, RG17, RG8, HBO4P, Basma Xanthi BX 2A, Coker 319, Hicks, McNair, 944 (MN 944), Burley 21, K149, Yaka JB 125/3, Kasturi Mawar, NC 297, Coker 371 Gold, Wislica, Simmaba, Turkish Samsun, AA37-1, B13P, F4 from the cross BU21×Hoja Parado, line 97, Samsun, PO1BU 64, CC 101, CC 200, CC 27, CC 301, CC 400, CC 500, CC 600, CC 700, CC 800, CC 900, Coker 176, Coker 319, Coker 371 Gold, Coker 48, CU 263, DF911, Galpao tobacco, GL 261-1, GL 350, GL 737, GL 939, GL 973, HB 04P, K 149, K 326, K 346, K 358, K 394, K 399, K 730, KT 200, KY 10, KY 14, KY 160, KY 17, KY 171, KY 907, KY 160, Little Crittenden, McNair 373, McNair 944, msKY 14.times.L8, Narrow Leaf Madole, NC 100, NC 102, NC 2000, NC 291, NC 297, NC 299, NC 3, NC 4, NC 5, NC 6, NC 606, NC 71, NC 72, NC 810, NC BH 129, OXFORD 207, ‘Perique’ tobacco, PM016, PMO21, PM092, PM102, PM132, PM204, PM205, PM215, PM216, PM217, PVH03, PVH09, PVH19, PVH50, PVH51, R 610, R 630, R 7-11, R 7-12, RG 17, RG 81, RG H4, RG H51, RGH 4, RGH 51, RS 1410, SP 168, SP 172, SP 179, SP 210, SP 220, SP G-28, SP G-70, SP H20, SP NF3, TN 86, TN 90, TN 97, TN D94, TN D950, TR (Tom Rosson) Madole, VA 309, VA 309, VA 359, Xanthi (Mitchell-Mor), KTRD#3 Hybrid 107, Bel-W3, 79-615, Samsun Holmes NN, F4 from cross N. tabacum BU21×N. tabacum Hoja Parado, line 97, KTRDC#2 Hybrid 49, KTRDC#4 Hybrid 110, Burley 21, BY-64, KTRDC#5 KY 160 SI, KTRDC#7 FCA, KTRDC#6 TN 86 SI, KY 8959, KY 9, KY 907, MD 609, NC 2000, PG 01, PG 04, P01, PO3, RG 11, RG 8, Speight G-28, VA 509, AS44, Banket A1, Basma Drama B84/31, Basma I Zichna ZP4/B, Basma Xanthi BX 2A, Batek, Besuki Jember, C104, Coker 347, Criollo Misionero, Delcrest, Djebel 81, DVH 405, Galpao Comum, HBO4P, Hicks Broadleaf, Kabakulak Elassona, Kasturi Mawar, Kutsage E1, KY 14×L8, KY 171, LA BU 21, McNair 944, NC 2326, NC 71, PVH 2110, Red Russian, Samsun, Saplak, Simmaba, Talgar 28, Turkish Samsun, Wislica, Yayaldag, NC 4, TR Madole, Prilep HC-72, Prilep P23, Prilep PB 156/1, Prilep P12-2/1, Yaka JK-48, Yaka JB 125/3, TI-1068, KDH-960, TI-1070, TW136, Samsun NN, Izmir, Karabalgar, Denizli, Basma, TKF 4028, L8, TKF 2002, TN90, GR141, Basma xanthi, GR149, GR153, Petit Havana or Xanthi NN.

Preferred breeding lines, varieties or cultivars of N. tabacum that are suitable for transient expression include but not are limited to PO2, AS44, Wislica, Simmaba, PM132, PM092, PM016, RG17, RG8, HBO4P, Basma Xanthi BX 2A, Coker 319, Hicks, McNair 944 (MN 944), Burley 21, K149, Yaka JB 125/3, PM102, NC 297, PMO21, AA37-1, B13P, F4 from the cross BU21×Hoja Parado, line 97, Samsun, P01, PM204, PM205, PM215, PM216 and PM217.

In still another specific embodiment, the invention relates to a plant or plant cells that comprise a vector of the invention and as defined in any one of the preceding embodiments, wherein said plant or plant cell is a Nicotiana tabacum plant variety, breeding line, or cultivar selected from the group consisting of Nicotiana tabacum line PM016, the seeds of which were deposited on 6 Jan. 2011 at NCIMB Ltd, (an International Depositary Authority under the Budapest Treaty, located at Ferguson Building, Craibstone Estate, Bucksburn, Aberdeen, AB21 9YA, United Kingdom) under accession number NCIMB 41798; PMO21, the seeds of which were deposited on 6 Jan. 2011 at NCIMB Ltd. under accession number NCIMB 41799; PM092, the seeds of which were deposited on 6 Jan. 2011 at NCIMB Ltd. under accession number NCIMB 41800; PM102, the seeds of which were deposited on 6 Jan. 2011 at NCIMB Ltd. under accession number NCIMB 41801; PM132, the seeds of which were deposited on 6 Jan. 2011 at NCIMB Ltd. under accession number NCIMB 41802; PM204, the seeds of which were deposited on 6 Jan. 2011 at NCIMB Ltd. under accession number NCIMB 41803; PM205, the seeds of which were deposited on 6 Jan. 2011 at NCIMB Ltd. under accession number NCIMB 41804; PM215, the seeds of which were deposited on 6 Jan. 2011 at NCIMB Ltd. under accession number NCIMB 41805; PM216, deposited under accession number NCIMB 41806; and PM217, the seeds of which were deposited on 6 Jan. 2011 at NCIMB Ltd. under accession number NCIMB 41807. Until the grant of a patent or for 20 years from the date of filing if the application is refused or withdrawn, a sample shall only be issued to an independent expert nominated by the requester (Rule 13bis.6 PCT).

In one embodiment of the invention, the invention provides the use of a vector molecule according to the present invention and as defined in any one of the preceding embodiments for the transfection of a bacterial cell or transformation of a plant or a plant cell and for expressing in said plant or plant cell a nucleic acid of interest.

In a specific embodiment of the invention, the expression of the nucleic acid of interest is directed to a specific plant tissue or a subcellular location.

In certain embodiments of the invention, the vector molecule according to the present invention and as defined in any one of the preceding embodiments is used for stable or transient expression of the gene of interest in a plant or plant cell, particularly a Nicotiana tabacum plant or plant cell, but especially in a plant or plant cell of any one of the Nicotiana tabacum plant varieties, breeding lines, or cultivars specified in the preceeding paragraphs. In particular, the vector molecule according to the invention and as defined in any one of the preceding embodiments is used for the generation of single copy transformation events without bacterial backbone sequences.

The invention also provides that the vector molecule be used for screening for promoter activity or function of a cryptic nucleic acid sequence.

The present application provides vector molecules and uses thereof which include the expression of one or more nucleic acids of interest in a plant or plant cell for the production of one or more proteins, metabolites or other compounds of interest, for regulating the expression of a nucleic acid of interest, for the identification of sequences with regulatory function in a plant cell, for the identification of gene and nucleic acid function, of either exogenous or endogenous nucleic acids, for directing tissue specific expression of a nucleic acid or protein of interest or for directing the expressed protein to a subcellular or extracellular location of a plant.

In one embodiment, the invention provides a vector molecule according to any of the preceding embodiments comprising an nucleic acid element containing a DNA fragment coding for a protein or polypeptide of interest for expression in a plant or plant cell. The protein or polynucleotide can be selected from the group consisting of growth factors, receptors, ligands, signaling molecules; kinases, enzymes, hormones, tumor suppressors, blood clotting proteins, cell cycle proteins, metabolic proteins, neuronal proteins, cardiac proteins, proteins deficient in specific disease states, antibodies andimmunoglobulins or a fragment thereof, antigens, proteins that provide resistance to diseases, antimicrobial proteins, interferons, and cytokines.

In one embodiment, the invention provides a vector molecule according to any of the preceding embodiments, wherein the fifth nucleic acid element further comprises, between the T-DNA right and T-DNA left border sequences, a regulatory element which is functional in a plant cell.

In one embodiment, the invention provides a vector molecule according to any of the preceding embodiments having a polynucleotide sequence being at least 80%, 81%, 82%, 83%, 84%, 85%, 86%, 87%, 88%, 89%, 90%, 91%, 92%, 93%, 94%, 95%, 96%, 97%, 98% or 99%, but particularly 100% identical to the polynucleotide sequence as depicted in SEQ ID NO: 1 and wherein the nucleic acid elements (a) to (e) exhibit the same functionality as the counterpart elements provided in SEQ ID NO:1.

In one embodiment, the invention provides a vector molecule according to any of the preceding embodiments, wherein the fifth nucleic acid element further comprises, between the T-DNA right and T-DNA left border sequences, a nucleotide sequence encoding a protein of interest which is operably linked to a regulatory element which is functional in a plant cell.

In a particular embodiment, the invention provides a vector molecule according to any of the preceding embodiments, wherein the nucleotide sequence encoding the protein of interest is influenza haemagglutinin 5 (H5), particularly influenza haemagglutinin 5 (H5) as shown in SEQ ID NO: 24.

In various embodiments, the invention provides methods for producing a protein of interest in a plant cell comprising introducing into a plant cell at least one vector according to any of the preceding embodiments, wherein the fifth nucleic acid element further comprises, between the T-DNA right and T-DNA left border sequences, a nucleotide sequence encoding a protein of interest which is operably linked to a regulatory element which is functional in a plant cell, particularly a plant cell of a plant of the genus Nicotiana. Also encompassed is the plant cell prepared according to the methods of the invention as described above.

### DEFINITIONS

Technical and scientific terms and expressions used within the scope of this application are generally to be given the meaning commonly applied to them in the pertinent art of plant biology. Reference is made herein to various methodologies known to those of skill in the art. Publications and other materials setting forth such known methodologies to which reference is made are incorporated herein by reference in their entireties as though set forth in full. The practice of the invention will employ, unless otherwise indicated, conventional techniques of chemistry, molecular biology, microbiology, genetic engineering and plant biology, which are within the skill of the art.

Any suitable materials and/or methods known to those of skill can be utilized in carrying out the present invention: however, preferred materials and/or methods are described. Materials, reagents and the like to which reference is made in the following description and examples are obtainable from commercial sources, unless otherwise noted.

All of the following term definitions apply to the complete content of this application. The word “comprising” does not exclude other elements or steps, and the indefinite article “a” or “an” does not exclude a plurality. A single step may fulfil the functions of several features recited in the claims. The terms “essentially”, “about”, “approximately” and the like in connection with an attribute or a value particularly also define exactly the attribute or exactly the value, respectively. The term “about” in the context of a given numerate value or range refers to a value or range that is within 20%, within 10%, or within 5% of the given value or range.

A “plant” as used within the present invention refers to any plant at any stage of its life cycle or development, and its progenies.

A “plant part” or “part of a plant” as used herein is meant to refer to any part of a plant, i.e. a plant organ, a plant tissue, a plant cell, an embryo, a leaf, etc. in planta or in culture. In certain embodiments of the invention relating to plant inoculation under high or low pressure or a combination thereof, this term refers to plant parts in planta.

A “tobacco plant” as used within the present invention refers to a plant of a species belonging to the genus Nicotiana, including but not limited to Nicotiana tabacum (or N. tabacum). Certain embodiments of the invention are described herein using the term “tobacco plant” without specifying Nicotiana tabacum, such descriptions are to be construed to have included Nicotiana tabacum specifically.

A “plant cell” or “tobacco plant cell” as used within the present invention refers to a structural and physiological unit of a plant, particularly a tobacco plant. The plant cell may be in form of a protoplast without a cell wall, an isolated single cell or a cultured cell, or as a part of higher organized unit such as but not limited to, plant tissue, a plant organ, or a whole plant.

“Plant material” as used within the present invention refers to any solid, liquid or gaseous composition, or a combination thereof, obtainable from a plant, including leaves, stems, roots, flowers or flower parts, fruits, pollen, egg cells, zygotes, seeds, cuttings, secretions, extracts, cell or tissue cultures, or any other parts or products of a plant.

“Plant tissue” as used herein means a group of plant cells organized into a structural or functional unit. Any tissue of a plant in planta or in culture is included. This term includes, but is not limited to, whole plants, plant organs, and seeds.

A “plant organ” as used herein relates to a distinct or a differentiated part of a plant such as a root, stem, leaf, flower bud or embryo.

The term “optical density” or “OD” relates to the optical determination of absorbance of an optical element at a given wavelength (e.g. 600 nm=OD₆₀₀) measured in a spectrophotometer.

The term “polynucleotide” is used herein to refer to a polymer of nucleotides, which may be unmodified or modified deoxyribonucleic acid (DNA) or ribonucleic acid (RNA). Accordingly, a polynucleotide can be, without limitation, a genomic DNA, complementary DNA (cDNA), mRNA, or antisense RNA. Moreover, a polynucleotide can be single-stranded or double-stranded DNA, DNA that is a mixture of single-stranded and double-stranded regions, a hybrid molecule comprising DNA and RNA, or a hybrid molecule with a mixture of single-stranded and double-stranded regions. In addition, the polynucleotide can be composed of triple-stranded regions comprising DNA, RNA, or both. A polynucleotide can contain one or more modified bases, such as phosphothioates, and can be a peptide nucleic acid (PNA). Generally, polynucleotides provided by this invention can be assembled from isolated or cloned fragments of cDNA, genome DNA, oligonucleotides, or individual nucleotides, or a combination of the foregoing.

The term “gene sequence” as used herein refers to the nucleotide sequence of a nucleic acid molecule or polynucleotide that encodes a protein or polypeptide, particularly a heterologous protein or polypeptide or a biologically active RNA, and encompasses the nucleotide sequence of a partial coding sequence that only encodes a fragment of a heterologous protein. A gene sequence can also include sequences having a regulatory function on expression of a gene that are located upstream or downstream relative to the coding sequence as well as intron sequences of a gene.

The term “transcription regulating nucleotide sequence” or “regulatory sequences”, each refer to nucleotide sequences influencing the transcription, RNA processing or stability, or translation of the associated (or functionally linked) nucleotide sequence to be transcribed. The transcription regulating nucleotide sequence may have various localizations with the respect to the nucleotide sequences to be transcribed. The transcription regulating nucleotide sequence may be located upstream (5′ non-coding sequences), within, or downstream (3′ non-coding sequences) of the sequence to be transcribed (e.g., a coding sequence). The transcription regulating nucleotide sequences may be selected from the group comprising enhancers, promoters, translation leader sequences, introns, 5′-untranslated sequences, 3′-untranslated sequences, and polyadenylation signal sequences. They include natural and synthetic sequences as well as sequences, which may be a combination of synthetic and natural sequences.

The term “promoter” refers to the nucleotide sequence at the 5′ end of a gene that directs the initiation of transcription of the gene. Generally, promoter sequences are necessary, but not always sufficient, to drive the expression of a gene to which it is operably linked. In the design of an expressible gene construct, the gene is placed in sufficient proximity to and in a suitable orientation relative to a promoter such that the expression of the gene is controlled by the promoter sequence. The promoter is positioned preferentially upstream to the gene and at a distance from the transcription start site that approximates the distance between the promoter and the gene it controls in its natural setting. As is known in the art, some variation in this distance can be tolerated without loss of promoter function. As used herein, the term “operatively linked” means that a promoter is connected to a coding region in such a way that the transcription of that coding region is controlled and regulated by that promoter. Means for operatively linking a promoter to a coding region are well known in the art.

The term “suppressor of gene silencing” used in the context of this invention refers to virus-encoded proteins that allow certain viruses to circumvent post-transcriptional gene silencing by binding to silencing RNA's. Also transgenes when introduced in a plant cell, can trigger post-transcriptional gene silencing as the result of which low or no expression of such genes is established.

The terms “protein”, “polypeptide”, “peptide” or “peptide fragments” as used herein are interchangeable and are defined to mean a biomolecule composed of two or more amino acids linked by a peptide bond, which may be folded into secondary, tertiary or quaternary structure to achieve a particular morphology.

The term “heterologous” as used herein refers to a biological sequence that does not occur naturally in the context of a specific polynucleotide or polypeptide in a cell or an organism. The term “recombinant protein” or “heterologous protein” or “heterologous polypeptide”, as used herein interchangeabky, refers to a protein or polypeptide that is produced by a cell but does not occur naturally in the cell. For example, the recombinant or heterologous protein produced in a plant cell or whole plant can be a mammalian or human protein.

The heterologous protein that can be expressed in a modified plant cell can be an antigen for use in a vaccine, including but not limited to a protein of a pathogen, a viral protein, a bacterial protein, a protozoal protein, a nematode protein; an enzyme, including but not limited to an enzyme used in treatment of a human disease, an enzyme for industrial uses; a cytokine; a fragment of a cytokine receptor; a blood protein; a hormone; a fragment of a hormone receptor, a lipoprotein; an antibody or a fragment of an antibody.

The terms “antibody” and “antibodies” refer to monoclonal antibodies, multispecific antibodies, human antibodies, humanized antibodies, camelised antibodies, chimeric antibodies, single-chain Fvs (scFv), single chain antibodies, single domain antibodies, domain antibodies (VH, VHH, VLA), Fab fragments, F(ab′) fragments, disulfide-linked Fvs (sdFv), and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “expressible” in the context of this invention refers to an operative linkage of a gene to regulatory elements that direct the expression of the protein or polypeptide encoded by the gene in plant cells comprised within a leaf.

The term “necrosis” and necrotic response” as used herein interchangeably relates to a hypersensitive response in the tissue of a plant, particularly a tobacco plant, triggered by, for example, inoculation of the plant tissue with, for example, an Agrobacterium strain. As a result, there is a poor survival rate of the target tissue. Necrosis is observed when injected leaf tissue has collapsed and cells died (see Klement & Goodman, Annual Review of Phytopathology 5 (1967) 17-44). Necrosis is distinguishable by one of ordinary skill in the art from yellowing as there is no collapse of the leaf tissue and no extensive cell death.

As used herein, a “T-DNA border” refers to a DNA fragment comprising an about 25 nucleotide long sequence capable of being recognized by the virulence gene products of an Agrobacterium strain, such as an A. fumefaciens or A. rhizogenes strain, or a modified or mutated form thereof, and which is sufficient for transfer of a DNA sequence to which it is linked, to eukaryotic cells, preferably plant cells. This definition includes, but is not limited to, all naturally occurring T-DNA borders from wild-type Ti plasmids, as well as any functional derivative thereof, and includes chemically synthesized T-DNA borders. In one aspect, the encoding sequence and expression control sequence of an expression construct according to the invention is located between two T-DNA borders.

The term “percent identity” or “sequence identity” in the context of two or more nucleotide sequences or amino acid sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

If two sequences which are to be compared with each other differ in length, sequence identity preferably relates to the percentage of the nucleotide residues of the shorter sequence which are identical with the nucleotide residues of the longer sequence. As used herein, the percent identity between two sequences is a function of the number of identical positions shared by the sequences (that is % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described herein below. For example, sequence identity can be determined conventionally with the use of computer programs such as the Bestfit program (Wisconsin Sequence Analysis Package, Version 8 for Unix, Genetics Computer Group, University Research Park, 575 Science Drive Madison, Wis. 53711). Bestfit utilizes the local homology algorithm of Smith and Waterman, Advances in Applied Mathematics 2 (1981), 482-489, in order to find the segment having the highest sequence identity between two sequences. When using Bestfit or another sequence alignment program to determine whether a particular sequence has for instance 95% identity with a reference sequence of the present invention, the parameters are preferably so adjusted that the percentage of identity is calculated over the entire length of the reference sequence and that homology gaps of up to 5% of the total number of the nucleotides in the reference sequence are permitted. When using Bestfit, the so-called optional parameters are preferably left at their preset (“default”) values. The deviations appearing in the comparison between a given sequence and the above-described sequences of the invention may be caused for instance by addition, deletion, substitution, insertion or recombination. Such a sequence comparison can preferably also be carried out with the program “fasta20u66” (version 2.0u66, September 1998 by William R. Pearson and the University of Virginia; see also W. R. Pearson (1990), Methods in Enzymology 183, 63-98). For this purpose, the “default” parameter settings may be used. Alternatively, the percentage identity of two sequences may be determined by comparing sequence information using the EMBOSS needle computer program (Rice et al. (2000) Trends in Genetics 16:276-277). EMBOSS needle reads two input sequences and writes their optimal global sequence alignment to file. It uses the Needleman-Wunsch alignment algorithm (Needleman and Wunsch (1970) J. Mol. Biol. 48: 443-453) to find the optimum alignment (including gaps) of two sequences along their entire length. The identity value is the percentage of identical matches between the two sequences over the reported aligned region (including any gaps in the length).

If the two nucleotide sequences to be compared by sequence comparison, differ in identity refers to the shorter sequence and that part of the longer sequence that matches the shorter sequence. In other words, when the sequences which are compared do not have the same length, the degree of identity preferably either refers to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that “matches” the shorter sequence. For example, nucleotide or amino acid sequences which are at least 80%, 81%, 82%, 83%, 84%, 85%, 86%, 87%, 88%, 89%, 90%, 91%, 92%, 93%, 94%, 95%, 96%, 97%, 98% or 99% identical to the polynucleotide sequence as depicted in SEQ ID NO: 1, may represent alleles, derivatives or variants of these sequences which preferably have a similar biological function. They may be either naturally occurring variations, for instance allelic sequences, sequences from other ecotypes, varieties, species, etc., or mutations. The mutations may have formed naturally or may have been produced by deliberate mutagenesis methods, such as those disclosed in the present invention. Furthermore, the variations may be synthetically produced sequences. The allelic variants may be naturally occurring variants or synthetically produced variants or variants produced by recombinant DNA techniques. Deviations from the above-described polynucleotides may have been produced, for example, by deletion, substitution, addition, insertion or recombination or insertion and recombination. The term “addition” refers to adding at least one nucleic acid residue or amino acid to the end of the given sequence, whereas “insertion” refers to inserting at least one nucleic acid residue or amino acid within a given sequence.

### Promoter/Enhancers/Terminators

The minimal binary vectors of the present invention according to any one of the preceding embodiments may contain, if desired, a promoter regulatory region (for example, one conferring inducible or constitutive, environmentally- or developmentally-regulated, or cell- or tissue-specific expression), a transcription initiation start site, a ribosome binding site, an RNA processing signal, a transcription termination site, and/or a polyadenylation signal. The regulatory elements to be used within the method of the invention may be part of an expression cassette and present in a vector molecule, particularly a binary vector, but especially a minimally sized binary vector as described herein, operably linked to a nucleotide sequence encoding a protein of interest

In various embodiments of the invention, the regulatory element is present in the T-DNA region of the minimally sized binary vector according to any one of the preceding embodiments as described herein.

Preferred promoters for use in the minimally sized binary vector according to any one of the preceding embodiments are cauliflower mosaic virus 35S promoter, a modified cauliflower mosaic virus 35S promoter, a double cauliflower mosaic virus 35S promoter, a minimal 35 S promoter, nopaline synthase promoter, a cowpea mosaic virus promoter, a HT-CPMV promoter, a tobacco copalyl synthase CPS2p promoter, a dihydrinin promoter, a plastocyanin promoter, a 35S/HT-CPMV promoter, and many other promoters that are derived from DNA viruses belonging to the Caulimoviridae family, either the full length transcript (FLt) promoters or the sub-genomic transcript promoters, examples of such DNA viruses including but not limited to cauliflower mosaic virus (CaMV), mirabilis mosaic virus (MMV), figwort mosaic virus (FMV), peanut chlorotic streak virus (PCISV). Particularly preferred for use in the minimally sized binary vector according to any one of the preceding embodiments is the full length transcript (FLt) promoters of DNA viruses belonging to the Caulimoviridae family including but not limited to FMV promoters, such as those described in WO1998000534 and US5994521, MMV promoters such as those describe in US6420547 and US6930182 and PCISV promoters such as those described in WO1998005198, US5850019 and EP929211. Many such promoters can be modified by linking multiple copies, for example two copies, of its enhancer sequence in tandem to enhance the promoter activity, such as but not limited to double CaMV 35S promoter (358×2), double MMV promoter (MMV×2), double FMV promoter (FMV×2). Functional fragments of these promoters known or described in the cited references can be used in the vector of the invention. Specific examples of such promoters have been created and EcoRI and HindIII restriction enzyme cleavage sites have been included at the ends to facilitate cloning into the minimal vectors of the invention. Nucleotide sequences that are at least 90%. 95%, 96%, 97%, 98%, 99% or 100% identical to these sequences and that are functional in enabling expression in plants of the operably linked nucleotide sequence can also be used in the vectors of the invention.

In a specific embodiment of the invention, one or more of the following promoter sequences may be used within a vector according to the invention and as described herein in any one of the preceding paragraphs:

pMMV single enhanced between EcoR1 and Hind3 sites
(SEQ ID NO: 25) [symbol]
gtcaacttcgtccacagacatcaacatcttatcgtcctttgaagataagataataa
tgttgaagataagagtgggagccaccactaaaacattgctttgtcaaaagctaaaaaagatg
atgcccgacagccacttgtgtgaagcatgagaagccggtccctccactaagaaaattagtga
agcatcttccagtggtccctccactcacagctcaatcagtgagcaacaggacgaaggaaatg
acgtaagccatgacgtctaatcccacaagaatttccttatataaggaacacaaatcagaagg
aagagatcaatcgaaatcaaaatcggaatcgaaatcaaaatcggaatcgaaatctctcatct

[symbol]

pMMV double enhanced between EcoR1 and Hind3 sites
(SEQ ID NO: 26) [symbol]
gtcaacttcgtccacagacatcaacatcttatcgtcctttgaagataagataataa
tgttgaagataagagtgggagcccccactaaaacattgctttgtcaaaagctaaaaaagatg
atgcccgacagccacttgtgtgaagcatgagaagccggtccctccactaagaaaattagtga
agcatcttccagtggtccctccactcacagctcaatcagtgagcaacaggacgaaggaaatg
acgtaagccatgacgtctaatcccaacttcgtccacagacatcaacatcttatcgtcctttg
aagataagataataatgttgaagataagagtgggagccaccactaaaacattgctttgtcaa
aagctaaaaaagatgatgcccgacagccacttgtgtgaagcatgagaagccggtccctccac
taagaaaattagtgaagcatcttccagtggtccctccactcacagctcaatcagtgagcaac
aggacgaaggaaatgacgtaagccatgacgtctaatcccacaagaatttccttatataagga
acacaaatcagaaggaagagatcaatcgaaatcaaaatcggaatcgaaatcaaaatcggaat
cgaaatctctcatct [symbol]
pFMV single enhanced between EcoR1 and Hind3 sites
(SEQ ID NO: 27) [symbol]
gtcaacatcgagcagctggcttgtggggaccagacaaaaaaggaatggtgcagaat
tgttaggcgcacctaccaaaagcatctttgcctttattgcaaagataaagcagattcctcta
gtacaagtggggaacaaaataacgtggaaaagagctgtcctgacagcccactcactaatgcg
tatgacgaacgcagtgacgaccacaaaagattgcccgggtaatccctctatataagaaggca
ttcattcccatttgaaggatcatcagatactcaaccaatatttctcactctaagaaattaag
agctttgtattcttcaatgagggctaagaccc [symbol]
pFMV double enhanced between EcoR1 and Hind3 sites
(SEQ ID NO: 28) [symbol]
gtcaacatcgagcagctggcttgtggggaccagacaaaaaaggaatggtgcagaat
tgttaggcgcacctaccaaaagcatctttgcctttattgcaaagataaagcagattcctcta
gtacaagtggggaacaaaataacgtggaaaagagctgtcctgacagcccactcactaatgcg
tatgacgaacgcagtgacgaccacaaaagattgcccaacatcgagcagctggcttgtgggga
ccagacaaaaaaggaatggtgcagaattgttaggcgcacctaccaaaagcatctttgccttt
attgcaaagataaagcagattcctctagtacaagtggggaacaaaataacgtggaaaagagc
tgtcctgacagcccactcactaatgcgtatgacgaacgcagtgacgaccacaaaagattgcc
cgggtaatccctctatataagaaggcattcattcccatttgaaggatcatcagatactcaac
caatatttctcactctaagaaattaagagctttgtattcttcaatgagaggctaagaccc

[symbol]

[symbol]

pPCISV single enhanced between EcoR1 and Hind3 sites
(SEQ ID NO: 29) [symbol]
aattcgtcaacgagatcttgagccaatcaaagaggagtgatgttgacctaaagcaa
taatggagccatgacgtaagggcttacgcccatacgaaataattaaaggctgatgtgacctg
tcggtctctcagaacctttactttttatatttggcgtgtatttttaaatttccacggcaatg
acgatgtgacctgtgcatccgctttgcctataaataagttttagtttgtattgatcgacacg
atcgagaagacacggccat [symbol]
pPCISV double enhanced between EcoR1 and Hind3 sites
(SEQ ID NO: 30) [symbol]
gtcaacgagatcttgagccaatcaaagaggagtgatgtagacctaaagcaataatg
gagccatgacgtaagggcttacgcccatacgaaataattaaaggctgatgtgacctgtcggt
ctctcagaacctttactttttatgtttggcgtgtatttttaaatttccacggcaatgacgat
gtgacccaacgagatcttgagccaatcaaagaggagtgatgtagacctaaagcaataatgga
gccatgacgtaagggcttacgcccatacgaaataattaaaggctgatgtgacctgtcggtct
ctcagaacctttactttttatatttggcgtgtatttttaaatttccacggcaatgacgatgt
gacctgtgcatccgctttgcctataaataagttttagtttgtattgatcgacacggtcgaga
agacacggccat

[symbol]

Two series of pC100-derived vectors were created by insertion of a FLt promoter from one of these DNA viruses from the Caulimoviridae family into the T-DNA region. FIG. 7 shows the T-DNA region of a series of nine vectors, namely pC141, pC190, pC191, pC192, pC193, pC241, pC242, pC243, and pC265. The multiple cloning site present downstream of the FLt promoter in these vectors allow the insertion of a nucleotide sequence of interest for expression in plant cells, particularly plant cells of plants of the genus Nicotiana, particularly Nicotina tabacum. A second series of smaller vectors was created by removing the expression cassette comprising the nucleotide sequence encoding the plant selectable marker (nptII) by digesting each of the vectors in the first series with SpeI and AvrII, and recircularizating the plasmid. These vectors, namely pC277, pC278, pC279, pC280, pC281 and pC282, are particularly suitable for transient expression of a polypeptide of interest in plant cells or plants, particularly plants of the genus Nicotiana, particularly Nicotiana tabacum. Accordingly, the binary vector of the invention as described herein in any one of the preceding embodiments may comprise in its T-DNA region, one or two or more copies of a FLt promoter of a DNA virus from MMV, FMV or PCISV, (e.g., SEQ ID NO: 25, SEQ ID NO: 26, SEQ ID NO: 27, SEQ ID NO: 28, SEQ ID NO: 29, SEQ ID NO: 30) and optionally an expression cassette comprising a nucleotide sequence encoding a plant selectable marker. In one embodiment, the minimally-sized binary vector of the invention as described herein in any one of the preceding embodiments may comprise one or more regulatory sequences derived from cowpea mosaic virus (HT-CPMV; WO 07/135,480 which is incorporated herein by reference in its entirety). Preferably, the binary vector also comprises the minimal 35S CaMV promoter. The HT-CPMV system is based on a minimal promoter, a modified 5′-UTR, containing hyper-translatable (HT) elements, and the 3′-UTR from CPMV RNA-2 which enables enhanced translation and high accumulation of recombinant proteins in plants.

minimal 35S-CaMV promoter
(SEQ ID NO: 20)
gaaacctcctcggattccattgcccagctatctgtcactttattgagaagatagtggaaaag
gaaggtggctcctacaaatgccatcattgcgataaaggaaaggccatcgttgaagatgcctc
tgccgacagtggtcccaaagatggacccccacccacgaggagcatcgtggaaaaagaagacg
ttccaaccacgtcttcaaagcaagtggattgatgtgatatctccactgacgtaagggatgac
gcacaatcccactatccttcgcaagacccttcctctatataaggaagttcatttcatttgga
gagg
5′UTR HT-CPMV
(SEQ ID NO: 21)
tattaaaatcttaataggttttgataaaagcgaacgtggggaaacccgaaccaaaccttctt
ctaaactctctctcatctctcttaaagcaaacttctctcttgtctttcttgcgtgagcgatc
ttcaacgttgtcagatcgtgcttcggcaccagtacaacgttttctttcactgaagcgaaatc
aaagatctctttgtggacacgtagtgcggcgccattaaataacgtgtacttgtcctattctt
gtcggtgtggtcttgggaaaagaaagattgctggaggctgctgttcagccccatacattact
tgttacgattctgctgactttcggcgggtgcaatatctctacttctgcttgacgaggtattg
ttgcctgtacttctttcttcttcttcttgctgattggttctataagaaatctagtattttct
ttgaaacagagttttcccgtggttttcgaacttggagaaagattgttaagcttctgtatatt
ctgcccaaatttgtcgggccc
3′UTR HT-CPMV
(SEQ ID NO: 22)
attttctttagtttgaatttactgttattcggtgtgcatttctatgtttggtgagcggtttt
ctgtgctcagagtgtgtttattttatgtaatttaatttctttgtgagctcctgtttagcagg
tcgtcccttcagcaaggacacaaaaagattttaattttattaaaaaaaaaaaaaaaagaccg
gg

The promoter sequence may consist of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements, derived from different promoters found in nature, or even be comprised of synthetic DNA segments. A promoter may also contain DNA sequences that are involved in the binding of protein factors which control the effectiveness of transcription initiation in response to physiological or developmental conditions.

Examples of enhancers include elements from the CaMV 35S promoter, octopine synthase genes (Ellis et al., 1987), the rice actin I gene, the maize alcohol dehydrogenase gene (Callis 1987), the maize shrunken I gene (Vasil 1989), tobacco etch virus (TEV) and tobacco mosaic virus (TMV) omega translation enhancers (Gallie 1989) and promoters from non-plant eukaryotes (e.g. yeast; Ma 1988). Vectors for use in accordance with the present invention may be constructed to include such an enhancer element. The use of an enhancer element, and particularly multiple copies of the element, may act to increase the level of transcription from adjacent promoters when applied in the context of plant transformation.

The termination region may be selected from the group consisting of a nopaline synthase (nos), a vegetative storage protein (vsp), or a proteinase inhibitor-2 (pint) termination region.

### Signal Peptides

The minimal binary vectors according to any one of the preceding embodiments may further comprise a nucleotide sequence encoding a signal peptide that targets the newly expressed protein to a subcellular location. Signal peptides that may be used within such vector molecules are, for example, those selected from a group consisting of a vacuolar targeting sequence, a chloroplast targeting sequence, a mitochondrial targeting sequence, a sequence that induces the formation of protein bodies in a plant cell or a sequence that induces the formation of oil bodies in a plant cell.

In one embodiment of the invention, the targeting sequence is a signal peptide for import of a protein into the endoplasmic reticulum. Signal peptides are transit peptides that are located at the extreme N-terminus of a protein and cleaved co-translationally during translocation across the endoplasmatic reticulum membrane. A signal peptide that can be used in a vector molecule according to the invention, without being limited thereto, is that naturally occurring at the N-terminus of a light or heavy chain sequence of an IgG, or the patatin signal peptide as described in EP2002807566 and WO2007EP1606, particularly the patatin signal peptide of pC148 as described in Example 3. Any nucleotide sequence that can encode the patatin signal peptide sequence can be used.

In one embodiment, a nucleotide sequence encoding the patatin signal peptide, wherein the patatin signal peptide consists of:

### MATTKSFLILFFMILATTSSTCA (SEQ ID NO: 31)

may be used within a vector according to the invention and as described herein in any one of the preceding embodiments.

Further signal peptides can, for example, be predicted by the SignalP prediction tool (Emanuelsson et al., 2007, Nature Protocols 2: 953-971).

In another embodiment of the invention, the targeting sequence may be an endoplasmatic reticulum retention peptide. Endoplasmatic reticulum retention targeting sequences occur at the extreme C-terminus of a protein and can be a four amino acid sequence such as KDEL, HDEL or DDEL, wherein K is lysine, D is aspartic acid, E is glutamic acid, L is leucine and H is histidine.

In still another embodiment of the invention, the targeting sequence may be a sequence that when fused to a protein results in the formation of non-secretory storage organelles in the endoplasmatic reticulum such as but not limited to those described in WO07/096,192, WO06/056483 and WO06/056484.

In certain embodiments of the invention, the targeting sequence can be a vacuolar targeting sequence, a chloroplast targeting sequence, a mitochondrial targeting sequence or any other sequence the addition of which results in a specific targeting of the protein fused there onto to a specific organelle within the plant or plant cell.

In one embodiment, the vector molecule according to the invention and as defined in any one of the preceding embodiments further comprises in the T-DNA region a site-specific recombination site for site-specific recombination.

In one embodiment, the site-specific recombination site is located downstream of the plant regulatory element. In another embodiment, the site-specific recombination site is located upstream of the plant regulatory element.

In a specific embodiment of the invention, the recombination site is a LoxP site and part of a Cre-Lox site-specific recombination system.

The Cre-Lox site-specific recombination system uses a cyclic recombinase (Cre) which catalyses the recombination between specific sites (LoxP) that contain specific binding sites for Cre.

In another specific embodiment, the recombination site is a Gateway destination site. For example, nucleic acids of interest are first cloned into a commercially available “entry vector” and subsequently recombined into a “destination vector”. The destination vector can be used for the analysis of promoter activity of a given nucleic acid sequence or number of sequences, for analysis of function, for protein localization, for protein-protein interaction, for silencing of a given gene or for affinity purification experiments.

### Suppressor of Gene Silencing

In various embodiments, the minimal binary vectors according to any one of the preceding embodiments may further comprise a suppressor of gene silencing, particularly a suppressor of gene silencing of viral origin, and particularly a suppressor of gene silencing of a potyvirus or a virus selected from the group consisting of Cucumber necrosis virus (CNV), Havel river virus (HaRV), Pear latent virus (PeLV), Lisianthus necrosis virus, Grapevine Algerian latent virus, Pelargonium necrotic spot virus (PeNSV), Cymbidium ringspot virus (CymRSV), Artichoke mottled crinkle virus (AMCV), Carnation Italian ringspot virus (CIRV), Lettuce necrotic stunt virus, Rice yellow mottle virus (RYMV), Potato virus X (PVX), Potato virus Y (PVY), African cassaya mosaic virus (ACMV), cucumber mosaic virus (CMV), Tobacco etch virus (TEV) or Tomato bushy stunt virus (TBSV).

In another embodiment said suppressor of gene silencing is selected from the group consisting of the p19 protein of cucumber necrotic virus (CNV), the p1 protein of rice yellow mottle virus (RYMV), the p25 protein of potato virus X (PVX), the AC2 protein of African cassaya mosaic virus (ACMV), the 2b protein of cucumber mosaic virus (CMV) and the helper-component proteinase (HcPro) of tobacco etch virus (TEV).

Detailed descriptions of suppressor of gene silencing including HcPro are provided in WO98/44097, WO 01/38512, and WO01/34822, which are incorporated herein by reference in their entirety. An exemplary nucleotide sequence encoding HcPro, herein referred to as P1-HcPro-P3 (SEQ ID NO: 23), can be inserted in a binary vector known in the art or a minimally-sized binary vector of the invention. Accordingly, in an non-limiting example, the expressible HcPro gene sequence comprises SEQ ID NO: 23 or a fragment thereof which is functional in enhancing the yield of heterologous protein in tobacco plant.

### Heterologous Protein

The minimal binary vectors according to any one of the preceding embodiments may further contain an expressible nucleotide sequence encoding a protein or polypeptide, particularly a heterologous protein or polypeptide, selected from the group consisting of growth factors, receptors, ligands, signaling molecules; kinases, enzymes, hormones, tumor suppressors, blood clotting proteins, cell cycle proteins, metabolic proteins, neuronal proteins, cardiac proteins, proteins deficient in specific disease states, antibodies, antigens, proteins that provide resistance to diseases, antimicrobial proteins, interferons, and cytokines. The expressible nucleotide sequence may comprises a sequence that has been optimized for expression in plant cells, particularly in plant cells of plants of the genus Nicotiana, particularly Nicotina tabacum. Although the expressible nucleotide sequence may be different from the native human coding sequence, the amino acid of the translated product is identical. One or more codons in the expressible nucleotide sequence have been replaced with preferred codons according to the known codon usage of plant, particularly a plant of the genus Nicotiana, particularly Nicotina tabacum, resulting in a pattern of preferred codons encoding the same amino acids in an expressible nucleotide sequence that enables increased expression in plant or tobacco plant (relative to using the native coding sequence). Techniques for modifying a nucleotide sequence for such purposes are well known, see for example, U.S. Pat. No. 5,786,464 and U.S. Pat. No. 6,114,148.

In one aspect, the binary vectors according to any one of the preceding embodiments may contain an antigen encoding sequences including sequences for inducing protective immune responses (e.g., as in a vaccine formulation). Such suitable antigens include but are not limited to microbial antigens (including viral antigens, bacterial antigens, fungal antigens, parasite antigens, and the like); antigens from multicellular organisms (such as multicellular parasites); allergens; and antigens associated with human or animal pathologies (e.g., such as cancer, autoimmune diseases, and the like). In one preferred aspect, viral antigens include, but are not limited to: HIV antigens; antigens for conferring protective immune responses to influenza; rotavirus antigens; anthrax antigens; rabies antigens; and the like. Vaccine antigens can be encoded as multivalent peptides or polypeptides, e.g., comprising different or the same antigenic encoding sequences repeated in an expression construct, and optionally separated by one or more linker sequences.

In one embodiment, the expressible nucleotide sequence encodes a light chain of an antibody, a heavy chain of an antibody, or both a light chain and a heavy chain of an antibody. In a specific embodiment, the heavy chain or light chain is that of an antibody that binds human CD20. In another specific embodiment, the heavy chain or light chain is that of an antibody that binds human CD20 with the antibody binding site of a rituximab.

In various embodiments, the expressible nucleotide sequence encodes a heterologous protein or polypeptide selected from the group consisting of an influenza virus antigen, particularly a haemagglutinin (HA). Influenza viruses are enveloped virus that bud from the plasma membrane of infected mammalian cells. They are classified into types A, B, or C, based on the nucleoproteins and matrix protein antigens present. Influenza type A viruses may be 15 further divided into subtypes according to the combination of hemagglutinin (HA) and neuraminidase (NA) surface glycoproteins presented. HA governs the ability of the virus to bind to and penetrate the host cell.

Currently, 16 HA (H1-H16) subtypes are recognized. Each type A influenza virus presents one type of HA and one type of NA glycoprotein. HA protein that can be produced by the methods of the invention include HI, H2, H3, H4, H5, H6, H7, H8, H9, H10, H11, H12, H13, H14, H15 or H16 or fragment or portion thereof. Examples of subtypes comprising such HA proteins include A/New Calcdonia/20/99 (H1N1), A/Indonesia/512006 (H5N1), A/chicken/New York/1995, A/herring gull/DE/677/88 (H2N8), A/Texas/32/2003, A/mallard/MN/33/00, A/duck/Shanghai/1/2000, A/northem pintail/TXI828189/02, A/Turkey/Ontario/6118/68(H8N4), A/shoveler/Iran/G54/03, A/chicken/Germany/N/1949(H10N7), A/duck/England/56(H11N6), A/duck/Alberta/60176(H12N5), A/Gull/Maryland/704/77(H13N6), A/Mallard/Gurjev/263/82, A/duck/Australia/341/83 (H15N8), A/black-headed gull/Sweden/5/99(H16N3), B/Lee/40, C/Johannesburg/66, A/PuertoRico/8/34 (H1N1), A/Brisbane/5912007 (H1N1), A/Solomon Islands 3/2006 (H1N1), A/Brisbane 10/2007 (H3N2), A/Wisconsin/6712005 (H3N2), B/Malaysia/2506/2004, B/Florida/4/2006, A/Singapore/1/57 (H2N2), A/Anhui/112005 (H₅N₁), A/Vietnam/1194/2004 (H5N1), A/Teal/HongKong/W312/97 (H6N1), A/Equine/Prague/56 (H7N7), A/HongKong/1073/99 (H9N2). It is contemplated that some of the influenza viruses having one of the above mentioned H subtypes can cause an infection in human, and because of its origin, can lead to a pandemic. Many of the antigens of these subtypes (H4, H5, H6, H7, H8, H9, H10, H1, H12, H13, H14, H15, H16) can thus be used in a pandemic influenza vaccine. The subtypes H1, H2, H3 are the major subtypes that are involved in human influenza infection and antigens of such subtypes are contemplated for use in a seasonal influenza vaccine.

It is contemplated that any nucleotide sequence that encodes an influenza haemagglutinin or an immunogenic fragment thereof can be used in the methods of the invention, such that the haemagglutinin polypeptide or a fragment thereof is produced in a host N. tabacum variety. For example, any of the biological sequences of influenza haemagglutinin reported in public databases, such as Genbank (Nucleic Acids Research 2004 Jan. 1; 32(1):23-6), or the Influenza Research Database (IRD; see www.fludb.oru or Squires et al. BioHealthBase: informatics support in the elucidation of influenza virus host pathogen interactions and virulence. Nucleic Acids Research (2008) vol. 36 (Database issue) pp. D497) can be used according to the present invention.

An example of a nucleotide sequence encoding a heterologous protein of interest is provided below as set forth in SEQ ID NO: 24. This nucleotide sequence encodes the mature influenza haemagglutinin 5 (H5). Accordingly, the invention contemplates vectors according to any one of the preceding embodiments as described above comprising, in the T-DNA region and operably linked to a plant regulatory element, a nucleotide sequence encoding a mature influenza haemaglutinin 5 exhibiting at least 90%, 92%, 94%, 96%, 98%, 99% or 99.5% sequence identity to SEQ ID NO: 24.

(SEQ ID NO: 24)
atggagaaaatagtgcttcttcttgcaatagtcagtcttgttaaaagtgatcagatttgcat
tggttaccatgcaaacaattcaacagagcaggttgacacaatcatggaaaagaacgttactg
ttacacatgcccaagacatactggaaaagacacacaacgggaagctctgcgatctagatgga
gtgaagcctctaattttaagagattgtagtgtagctggatggctcctcgggaacccaatgtg
tgacgaattcatcaatgtaccggaatggtcttacatagtggagaaggccaatccaaccaatg
acctctgttacccagggagtttcaacgactatgaagaactgaaacacctattgagcagaata
aaccattttgagaaaattcaaatcatccccaaaagttcttggtccgatcatgaagcctcatc
aggagttagctcagcatgtccatacctgggaagtccctccttttttagaaatgtggtatggc
ttatcaaaaagaacagtacatacccaacaataaagaaaagctacaataataccaaccaagag
gatcttttggtactgtggggaattcaccatcctaatgatgcggcagagcagacaaggctata
tcaaaacccaaccacctatatttccattgggacatcaacactaaaccagagattggtaccaa
aaatagctactagatccaaagtaaacgggcaaagtggaaggatggagttcttctggacaatt
ttaaaacctaatgatgcaatcaacttcgagagtaatggaaatttcattgctccagaatatgc
atacaaaattgtcaagaaaggggactcagcaattatgaaaagtgaattggaatatggtaact
gcaacaccaagtgtcaaactccaatgggggcgataaactctagtatgccattccacaacata
caccctctcaccatcggggaatgccccaaatatgtgaaatcaaacagattagtccttgcaac
agggctcagaaatagccctcaaagagagagcagaagaaaaaagagaggactatttggagcta
tagcaggttttatagagggaggatggcagggaatggtagatggttggtatgggtaccaccat
agcaatgagcaggggagtgggtacgctgcagacaaagaatccactcaaaaggcaatagatgg
agtcaccaataaggtcaactcaatcattgacaaaatgaacactcagtttgaggccgttggaa
gggaatttaataacttagaaaggagaatagagaatttaaacaagaagatggaagacgggttt
ctagatgtctggacttataatgccgaacttctggttctcatggaaaatgagagaactctaga
ctttcatgactcaaatgttaagaacctctacgacaaggtccgactacagcttagggataatg
caaaggagctgggtaacggttgtttcgagttctatcacaaatgtgataatgaatgtatggaa
agtataagaaacggaacgtacaactatccgcagtattcagaagaagcaagattaaaaagaga
ggaaataagtggggtaaaattggaatcaataggaacttaccaaatactgtcaatttattcaa
cagtggcgagttccctagcactggcaatcatgatggctggtctatctttatggatgtgctcc
aatggatcgttacaatgcagaatttgcatttaa

The construction and further details of the vectors according to the invention are described in the following examples, which serve to further illustrate the present invention but are not intended to be limiting.

The vectors of the invention are constructed by combining two parts, a first part containing structural and functional elements necessary for the replication and stable maintenance of the vector in a bacterial host cell, referred to herein as backbone vector or backbone sequence, and a second part containing structural elements for the delivery of a nucleic acid of interest to a plant cell, and referred to herein as transfer-DNA or T-DNA region. Nucleic acids for expression in a plant cell are added to the T-DNA region of the vector which is bordered by two direct repeat sequences, namely T-DNA right border and T-DNA left border.

For the purpose of cloning and especially for high throughput cloning of multiple nucleic acids into the T-DNA of such vectors, it is desirable that vectors for Agrobacterium-mediated transformation of plant cells can replicate in a host cell of Escherichia coli as well as in an Agrobacterium ssp host cell. The Agrobacterium spp. host cell can be Agrobacterium tumefaciens or Agrobacterium rhizogenes host cell. For the purpose of efficiency and ease of cloning of a nucleic acid into the T-DNA of such a vector, it is beneficial that such a vector is of minimum size and stably maintained as a high-copy plasmid. High-copy vectors for transforming plant cells are known but are still of considerably larger size (greater than 6000 basepairs) and tend to give rise to multiple and sometimes complex integrations into the plant nuclear genome. Multiple and complex integrations into the plant nuclear genome are not desirable as they result in post-transcriptional silencing of the gene that is transferred into the plant cell. In addition, such vectors also lead to integration of vector backbone sequences which cause a regulatory hurdle with the USDA and FDA for accepting such plants for the production of proteins or other compounds.

As exemplified in Example 1, a binary vector of less than 5,150 basepairs comprising a minimal backbone and T-DNA region is provided without affecting replication and stable maintenance in Escherichia coli and Agrobacterium spp as a high-copy plasmid.

As described in Examples 3 and 4, respectively, the use of the minimal binary vector pPMP1 (sequence of pPMP1 is provided in Table 1) and derivatives thereof resulted in stable as well as transient expression of nucleic acids, proteins or peptides in transformed plant cells of Nicotiana tabacum and Nicotiana benthamiana. Moreover, transformation with pPMP1 and derivatives thereof such as the minimal plant selectable binary pC100 vector, resulted preferably in single- or otherwise low-copy number integrations in the plant nuclear genome as exemplified in Example I and little or no integration of vector backbone sequences as exemplified in Example 5.

The present application therefore provides vectors for Agrobacterium-mediated transformation, particularly advantageous for the expression of a nucleic acid in a plant cell, in particular for expressing a protein or polypeptide of interest in a plant cell, plant tissue or specific compartment of a plant cell, for the production of one or more metabolites or other compounds of interest in a plant cell, or part of a plant cell, for regulating the expression of a nucleic acid of interest, for the identification of sequences with regulatory function in a plant cell, for the identification of gene and nucleic acid function, of either one or more exogenous or endogenous nucleic acids of interest.

These vectors are particularly advantageous since they are of minimal size, stably maintained as a high copy number in a bacterial cell, highly flexible and useful for multiple purposes and can be used for the expression of nucleic acids and proteins or polypeptides of interest in a stable transgenic plant or plant cell, or for the transient expression thereof.

### BRIEF DESCRIPTION OF SEQUENCES AND FIGURES

In the description and examples reference is made to the following sequences that are represented in the sequence listing:

SEQ ID NO: 1: depicts the nucleotide sequence of minimal binary pPMP1 vector.

SEQ ID NO: 2: depicts the nucleotide sequence of PQ24F forward primer for nptII gene.

SEQ ID NO: 3: depicts the nucleotide sequence of PQ24R reverse primer for nptII gene.

SEQ ID NO: 4: depicts the nucleotide sequence of Taqman probe for nptII gene.

SEQ ID NO: 5: depicts the nucleotide sequence of PQ17F forward primer for nitrate reductase gene.

SEQ ID NO: 6: nucleotide sequence of PQ17R reverse primer for tobacco nitrate reductase gene.

SEQ ID NO: 7: depicts the nucleotide sequence of Taqman probe for nitrate reductase gene.

SEQ ID NO: 8: depicts the nucleotide sequence of PC201F forward primer for pCambia-2300.

SEQ ID NO: 9: depicts the nucleotide sequence of PC202R reverse primer for pCambia-2300.

SEQ ID NO: 10: depicts the nucleotide sequence of primer 1 located at position −18 to −1 relative to the T-DNA left border of pC100.

SEQ ID NO: 11: depicts the nucleotide sequence of primer 2 located at position +2 to +25 relative to the T-DNA left border of pC100 SEQ ID NO: 12: depicts the nucleotide sequence of primer 3 located at position +122 to +137 downstream of the T-DNA left border of pC100.

SEQ ID NO: 13: depicts the nucleotide sequence of primer 4 located at position +264 to +232 downstream of the T-DNA left border on the bottom strand of pC100.

SEQ ID NO: 14: depicts the nucleotide sequence of primer 5 located at position −870 to −848 on the upper strand upstream of the T-DNA right border of pC100.

SEQ ID NO: 15: depicts the nucleotide sequence of primer 6 located at position −171 to −151 upstream of the T-DNA right border sequence of pC100.

SEQ ID NO: 16: depicts the nucleotide sequence of primer 7 located at position −26 to −1 on the bottom strand and relative to the T-DNA right border sequence of pC100.

SEQ ID NO: 17: depicts the nucleotide sequence of primer 8 located at position +87 to +102 downstream of the T-DNA right border sequence of pC100.

SEQ ID NO: 18: depicts the nucleotide sequence of primer 9 located on the upper strand at the amino terminus of the NPTII gene of pC100.

SEQ ID NO: 19: depicts the nucleotide sequence of primer 10 located on the bottom strand at the carboxy terminus of the NPTII gene of pC100.

SEQ ID NO: 20: depicts the nucleotide sequence of the minimal 35S-CaMV promoter

SEQ ID NO: 21: depicts the nucleotide sequence of the 5′UTR HT-CPMV

SEQ ID NO: 22: depicts the nucleotide sequence of the 3′UTR HT-CPMV

SEQ ID NO: 23: depicts the nucleotide sequence of P1-HcPro-P3

SEQ ID NO: 24: depicts the nucleotide sequence of influenza haemagglutinin 5 (H5)

SEQ ID NO: 25: depicts the nucleotide sequence of pMMV single enhanced promoter fragment between EcoR1 and Hind3 sites

SEQ ID NO: 26: depicts the nucleotide sequence of pMMV double enhanced promoter fragment between EcoR1 and Hind3 sites

SEQ ID NO: 27: depicts the nucleotide sequence of pFMV single enhanced promoter fragment between EcoR1 and Hind3 sites

SEQ ID NO: 28: depicts the nucleotide sequence of pFMV double enhanced promoter fragment between EcoR1 and Hind3 sites

SEQ ID NO: 29: depicts the nucleotide sequence of pPCISV single enhanced promoter fragment between EcoR1 and Hind3 sites

SEQ ID NO: 30: depicts the nucleotide sequence of pPCISV double enhanced promoter fragment between EcoR1 and Hind3 sites

SEQ ID NO: 31: depicts the amino acid sequence of the patatin signal peptide

SEQ ID NO: 32: depicts the nucleotide sequence of rituximab mature heavy chain (tobacco optimized) sequence as in C148

SEQ ID NO: 33: depicts the amino acid sequence of rituximab mature heavy chain (tobacco optimized) sequence as in C148

SEQ ID NO: 34: depicts the patatin tobacco non optimized sequence (slightly modified) as in C148 (in front of heavy chain)

SEQ ID NO: 35: depicts the nucleotide sequence of rituximab mature light chain (tobacco optimized) sequence as in C148

SEQ ID NO: 36: depicts the amino acid sequence of rituximab mature light chain (tobacco optimized) sequence as in C148

SEQ ID NO: 37: depicts the amino acid sequence of the patatin tobacco optimized sequence as in C148 (in front of light chain)

SEQ ID NO: 38: depicts the nucleotide sequence of mature GBA (tobacco optimized) sequence as delivered by synthesis

SEQ ID NO: 39: depicts the amino acid sequence of mature GBA

SEQ ID NO: 40: depicts the nucleotide sequence of tobacco-optimized patatin signal peptide in front of GBA

In the description and examples reference is made to the following figures:

FIG. 1 shows schematic diagrams of the minimal plant selectable binary vector pC100 (A) and the minimal binary vector pPMP1 (B) and a linear representation of pPMP1 (C). See also Example 1.

FIG. 2 shows the nucleotide sequence of linearized pPMP1 as in FIG. 1C and Example 1.

FIG. 3 shows an SDS-PAGE gel (A) of tobacco produced H5 and Western blot (B). See also Example 4.

FIG. 4 shows a Blue Native-PAGE gel (A) of tobacco produced H5 and Western blot (B). See also Example 4.

FIG. 5 shows a haemagglutination assay of tobacco produced H5 and purified H5. See also Example 4.

FIG. 6 shows a detailed overview of the T-DNA region of pC100 minimal plant selectable binary vector and location of primers 1 to 10 used to determine the integration of vector backbone sequences in transgenic plants at the left and right T-DNA border junctions. See also Example 5.

FIG. 7 shows schematic representation of the T-DNA region of the pC100-derived vectors (not to scale). LB is T left border; RB is T right border; pMMV is the FLt promoter of Mirabilis mosaic virus; pFMV, FLt promoter of Figwort mosaic virus; pPCISV, FLt promoter of Peanut chlorotic streak virus; CaMV 35S FLt promoter of Cauliflower mosaic virus; Plastocyanin, plant promoter isolated from alfalfa. MCS, multiple cloning site carrying HindiIII and SnaBI restriction sites; t35S: terminator 35S; tPlasto, terminator Plastocyanin, pNOS: nopaline synthase promoter, tNOS: nopaline synthase terminator; and nptII: neomycin phosphotransferase, plant kanamycin resistance gene. 2× (or ×2 used interchangeably) refer to the presence of two enhancer sequences.

FIG. 8 shows the results of comparing the level of H5 expression using different regulatory elements in the minimal vector. To facilitate identification and labeling of samples and figures, a letter followed by a number is used to designate a DNA vector infiltrated into an Agrobacterium strain, e.g. A100 corresponds to strain AGL1 transformed with construct C100.

### EXAMPLES

The following examples are provided as an illustration and not as a limitation. Unless otherwise indicated, the present invention employs conventional techniques and methods of molecular biology, cell biology, genomics, recombinant DNA technology and plant biology and plant breeding. Standard methodologies are described in e.g. Sambrook et al. (1989) Molecular cloning: a laboratory manual. Cold Spring Harbor Laboratory Press, 2^{nd} edition or Sambrook and Russel, 2001. Molecular cloning, a laboratory manual, 3^{td} edition, Cold Spring Harbor Laboratory Press, New York, USA. Ausubel et al. (2002) Short protocols in molecular biology, 5^{th} edition. MacPherson et al. (1995) PCR 2: a practical approach. Oxford University Press., unless otherwise indicated.

### Example 1

### Development of pPMP1 Minimal Binary Vector and Minimal Plant Selectable pC100 Binary Vector

### 1.1 Construction of T-DNA Region and Backbone Fragment.

Polynucleotides comprising the T-DNA region and the backbone sequence of the vector of the invention were synthesized chemically. A first fragment comprises a T-DNA region bordered by a T-DNA right (RB) and T-DNA left (LB) border sequence, a plant selectable kanamycin resistance (nptII) gene of pBIN61 (a vector of about 13,500 basepairs, Bendahmane et al., 2000. Plant Journal 21: 73-81) operably linked to a nopaline synthase (pNOS) promoter and a tNOS terminator, and unique StuI, AscI and EcoRI restriction sites which were flanked by PvuII restriction sites. This first fragment was cloned in the PvuII site of the pUC-derived pMK vector (Geneart, Regensburg, Germany) which contained a ColE1 replication of origin (Col E1 ori) and bacterial kanamycin resistance gene (KmR). The resulting vector was named pGA13.

A second fragment comprises backbone sequences which include a ColE1 origin of replication and a minimal RK2 oriV origin of replication, and a gene coding for the RK2-derived TrfA replication initiator protein of pBIN61. This second fragment was chemically synthesized with unique AscI, StuI and PvuII restriction sites and cloned in the pUC-derived pMA vector (Geneart, Regensburg, Germany) which also contained an ampicillin (ApR) resistance gene. The resulting vector was named pGA14.

1.2 Construction of pC100 Minimal Plant Selectable Binary Vector.

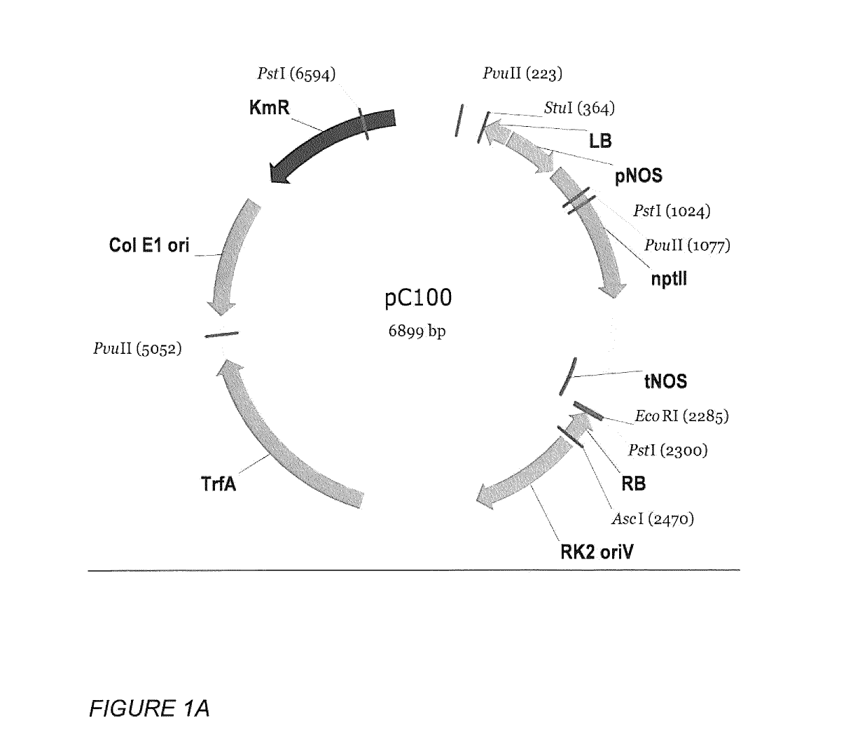

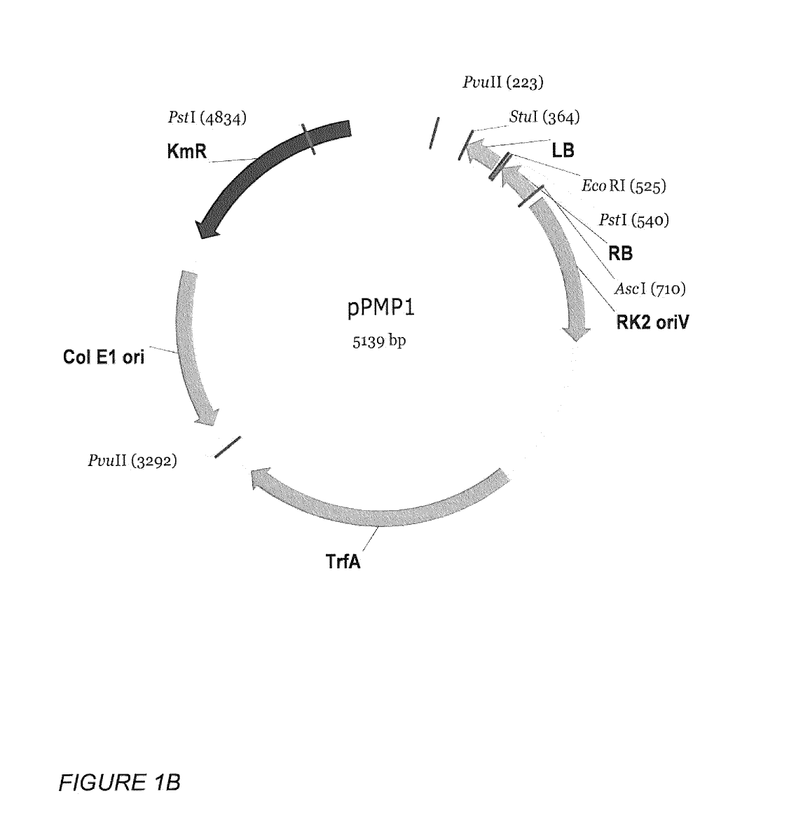

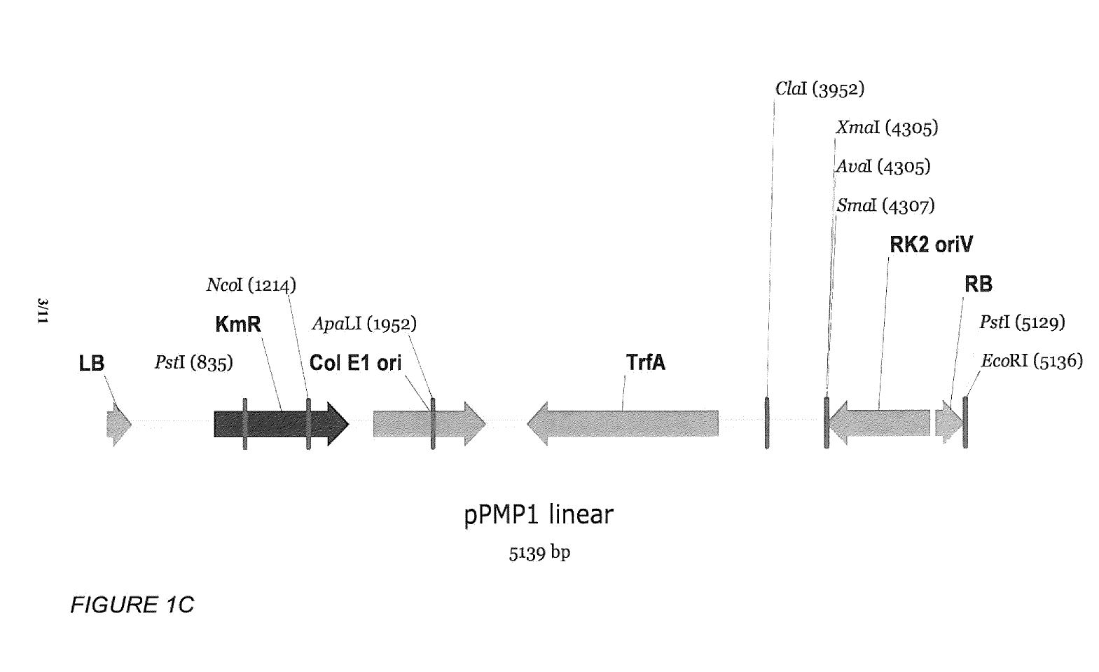

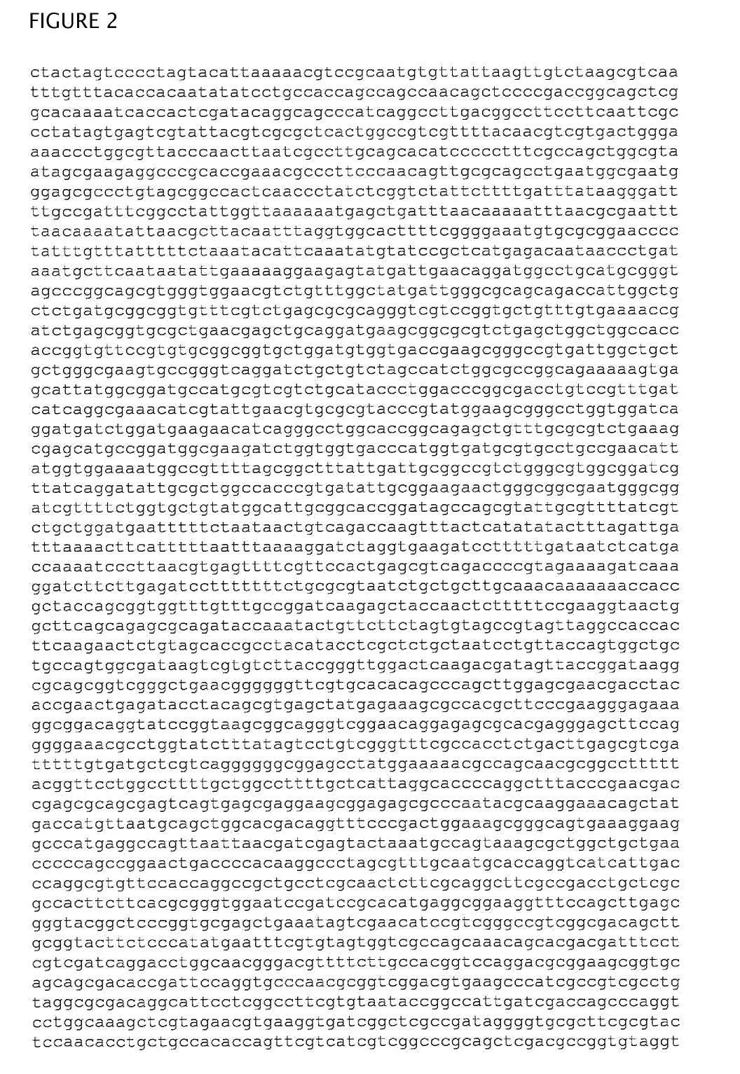

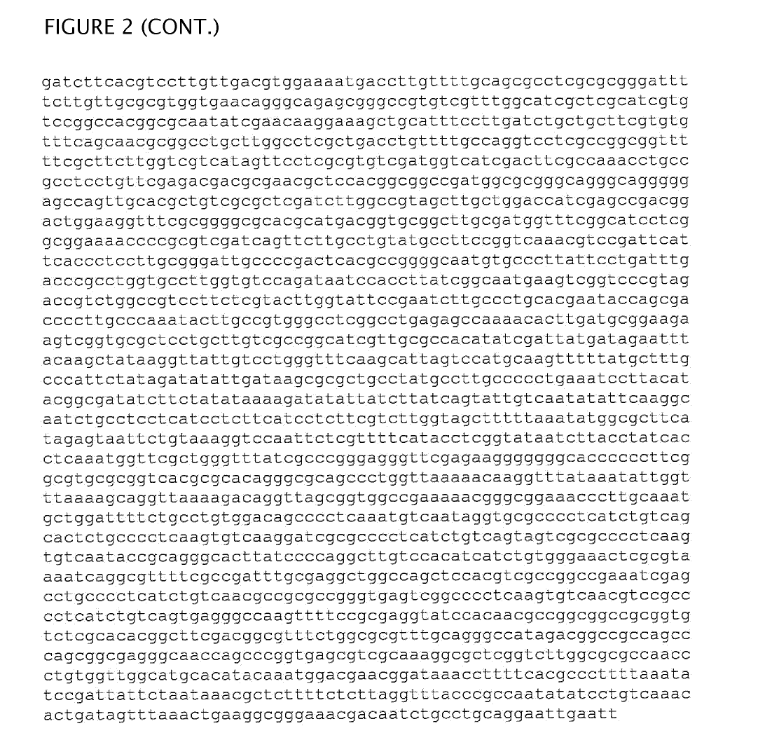

pC100 (FIG. 1A) was made by cloning the T-DNA region of pGA13 as an AscI-StuI fragment into the part of pGA14 vector that comprises the backbone sequence and that has been digested with AscI and StuI.

### 1.3 Construction of DPMP1 Minimal Binary Vector.

pPMP1 (5139 bp; SEQ ID NO: 1; FIG. 1B) was constructed by deleting the plant selectable nptII gene from pC100. A linear representation of pPMP1 starting with the unique EcoRI restriction site (position +1) upstream of LB, is shown in FIG. 1C. From left to right in the linear representation, pPMP1 comprises a unique EcoRI restriction site at position +1; a LB at position +69 to +94; a first gap sequence of 250 bp wherein the gap sequence has no function in replication of pPMP1, maintenance in a bacterial cell, or transfer of the T-DNA region to a plant cell; a first sequence of approximately 1100 bp containing a KmR gene coding sequence from +653 to +1454 and approximately 300 bp of regulatory sequences upstream and downstream of the coding sequence; a second gap sequence of approximately 150 bp; a second sequence containing a ColE1 on from +1602 to +2269; a third gap sequence of approximately 150 bp; a third sequence of approximately 1500 bp containing the coding sequence of TrfA from +3662 to +2517 and approximately 350 bp of regulatory sequences upstream and downstream of the coding sequence; a fourth gap sequence of approximately 450 bp; a fourth sequence containing an RK2 oriV from +4932 to 4303; a fifth gap sequence of 109 bp; a RB at position 5041 to 5066 and a unique EcoRI restriction site at position +5139.

### Example 2

### Efficiency of Transformation of Tobacco and Copy Number

### 2.1 Transformation of Tobacco.

pC100 and the commonly used pBINPLUS binary vector (Van Engelen et al., 1995. Transgenic Res. 4: 288-290) were introduced in two Agrobacterium tumefaciens strains, AV and LBA4404. Both pBINPLUS and pC100 contained a kanamycin resistance gene for selection of transgenic plant cells. Bacteria were grown overnight in liquid broth containing the appropriate antibiotics and during the following day, bacterial cells were collected by centrifugation, and resuspended in water. The density was adjusted to an OD₆₀₀ₙₘ=1 by dilution in water. Leaf explants of aseptically grown Nicotiana tabacum plants were transformed according to standard methods and co-cultivated for two days on medium according to Murashige & Skoog (1962. Physiol. Plant 15: 473-497) supplemented with 20 g/L sucrose and 8 g/L purified agar in a Petri dish under appropriate conditions known in the art. After two days of co-cultivation, explants were placed on selective medium containing kanamycin for selection of transformation, 250 mg/L vancomycin, 250 mg/L cefotaxim, 0.1 mg/L NAA and 1 mg/L BAP hormones for the regeneration of transgenic shoots. Kanamycin resistant tobacco plants were regenerated according to standard protocols.

### 2.2 Transformation Efficiency.

The number of tobacco shoots that rooted in selective medium containing 100 mg/L kanamycin sulfate after Agrobacterium-mediated transformation with pBINPLUS and pC100 was established as well as the T-DNA copy number in transgenic tobacco plants obtained from these shoots. The results summarized in Table 1 shows that in two independent transformation experiments the efficiency of transformation for pC100 was 55% and 44% respectively for Agl1 and 47% and 24% for LBA4404, compared to 30% and 26%, and 52% and 18% for pBINPLUS, respectively.

### 2.3 T-DNA Copy Number.

The T-DNA copy number of (i) 170 independent transgenic plants obtained after transformation using pC100 and (ii) 121 independent transgenic plants derived upon transformation using pBINPLUS was established using primers for the NPTII kanamycin resistance gene present on the T-DNA of both binary vectors (Table 2). As an internal control and for normalisation, the tobacco nitrate reductase gene (NIA) was used. Quantitative real-time Q-PCR was performed using the ABsolute™ QPCR Low ROX Mix (Axonlabs, AB-1319/A) and optical tube strips and caps from ABI (Applied Biosystems part n^{o} 4316567 and n^{o} 4323032) on a Mx3005p (Stratagene). Concentrations and PCR conditions were as follows: 12 μl of ABsolute™ QPCR Low ROX Mix, 2 μl of 5 μM of each primer (see Table 2 for details), 1 μl of 5 μM of each probe (see Table 2) and 2 μl of genomic DNA (100 ng), 15 min at 95° C., and 50 cycles with 30 sec at 95° C. and 1 min at 60° C. Amplicons of NPTII and NIA were amplified in the same well. Each sample was assayed in triplicate and analyzed with the MxPro software (Ingham et al., 2001, Biotechniques 31: 132-140) and Microsoft Excel. 74 out of 170 (44%) of the pC100 plants had a single copy T-DNA insertion compared to 47 out of 121 plants for pBINPLUS (39%; see Table 3).

TABLE 1
Transformation efficiency of pBINPLUS and pC100 using
Agrobacterium tumefaciens Agl1 or LBA4404 transformation
of leaf explants as measured in two independent transformation
experiments.

| Strain | Construct | Exp. | Explants | Plantlets | Efficiency % |
|---|---|---|---|---|---|
| Agl1 | pBINPLUS | 1 | 98 | 30 | 31 |
| | | 2 | 100 | 26 | 26 |
| | pC100 | 1 | 98 | 54 | 55 |
| | | 2 | 100 | 44 | 44 |
| LBA4401 | pBINPLUS | 1 | 91 | 47 | 52 |
| | | 2 | 100 | 18 | 18 |
| | pC100 | 1 | 90 | 47 | 52 |
| | | 2 | 100 | 24 | 24 |

Exp., experiment;
Explants, number of explants originally used and
Plantlets, number of kanamycin resistant plantlets obtained upon transformation and selection.

TABLE 2
Primer polynucleotide sequences for nitrate reductase
(NIA) and neomycin phosphotransferase II (kanamycin
resistance) gene.

| | Primer, probe | | | |
|---|---|---|---|---|
| Gene | (SEQ ID) | Sequence (5′-3′) | Dye | Quencher |
| NIA | PQ17F (SEQ ID NO: 5) | GGAAAGAACAGAACATGGTTAAACAA | | |
| | PQ17R (SEQ ID NO: 6) | ACACCGTACCGTTTTAACAAAGC | | |
| | PQ17 (SEQ ID NO: 7) | TGCCGCTGCCGTTTCAACAACTG | 5′HEX | 3′TAMRA |
| nptii | PQ24F SEQ ID NO: 2) | AGCTGTGCTCGACGTTGTCA | | |
| | PQ24R (SEQ ID NO: 3) | CCCCGGCACTTCGCCCAATA | | |
| | PQ24 (SEQ ID NO: 4) | TGAAGCGGGAAGGGACTGGC | 5′FAM | 3′TAMRA |

TABLE 3
Number of T-DNA copies in transgenic
kanamycin resistant plants.

T-DNA
copies

| Construct | 1 | ≧2 | Plants |
|---|---|---|---|
| pBINPLUS | 47 | 74 | 121 |
| pC100 | 74 | 96 | 170 |

### Example 3

### Transient Expression of Rituximab Monoclonal Antibody

### 3.1 Construction of Expression Vectors for Making Rituximab Monoclonal Antibody.

Rituxumab is a murine/human chimeric monoclonal IgG1 antibody that binds human CD20. Rituximab is used in the treatment of many lymphomas, leukemias, transplant rejection and some autoimmune disorders. An expression cassette comprising the full length-coding sequences of the rituximab monoclonal antibody light chain and heavy chain (CAS registry number 174722-31-7 or WO02/060955) was made by chemical synthesis with the choice of codons in the coding sequence being optimized for expression in a tobacco plant.

> rituximab mature heavy chain (tobacco optimized)
sequence as in C148
(SEQ ID NO: 32)
caagttcaacttcaacaaccaggtgctgaacttgttaagcctggtgcttctgttaagatgtc
ttgcaaggcttctggatacactttcacatcctacaacatgcattgggttaagcaaactccag
gacgtggacttgaatggattggagctatctaccctggaaacggtgatacttcctacaaccag
aagttcaagggaaaggctactcttactgctgataagtcctcttccactgcttacatgcaact
ttcttcactcacttccgaggattctgctgtttattactgcgctaggtccacttattatggtg
gagattggtacttcaatgtttggggagctggaactactgttactgtgtctgctgcttctact
aagggaccatctgtttttccacttgctccatcttctaagtctacttccggtggaactgctgc
tcttggatgccttgtgaaggattatttcccagagccagtgactgtttcttggaactctggtg
ctcttacttctggtgttcacactttcccagctgttcttcagtcatctggactttactccctt
tcttctgttgttactgtgccatcttcttcacttggaactcagacttacatctgcaacgttaa
ccacaagccatctaacacaaaagtggataagaaggcagagccaaagtcttgtgataagactc
atacttgtccaccatgtccagctccagaacttcttggtggtccatctgttttcttgttccca
ccaaagccaaaggatactctcatgatctctaggactccagaagttacttgcgttgttgtgga
tgtttctcatgaggacccagaggttaagttcaactggtacgtggatggtgttgaagttcaca
acgctaagactaagccaagataggaacagtacaactctacttaccgtgttgtgtctgtgctt
actgttcttcaccaggattggcttaacggaaaagagtacaaatgcaaggtttccaataaggc
tttgccagctccaattgaaaagactatctccaaggcaaaaggacagcctagagagccacagg
tttacactcttccaccatctagagatgagcttactaagaaccaggtttcccttacttgtctt
gtgaagggattctacccatctgatattgctgttgagtgggagtcaaacggacagcctgagaa
caactacaagactactccaccagtgcttgattctgatggttccttcttcctctactccaaac
tcactgtggataagtctagatggcagcagggaaatgttttctcttgctccgttatgcatgag
gctctccataatcactacactcagaagtccctttctttgtctcctggaaagtga
(SEQ ID NO: 33)
QVQLQQPGAELVKPGASVKMSCKASGYTFTSYNMHWVKQTPGRGLEWIGAIYPGNGDTSYNQ
KFKGKATLTADKSSSTAYMQLSSLTSEDSAVYYCARSTYYGGDWYFNVWGAGTTVTVSAAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKAEPKSCDKTHTCPPCPAPELLGGPSVFLFP
PKPKDTLMISRTPEVTCVVVDVSHEDPEVKFNWYVDGVEVHNAKTKPR*EQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK*

The mature heavy chain sequence was synthesized with a patatin signal peptide placed under control of the HT-CPMV promoter and HT-CPMV untranslated 5′ and 3′ UTR sequences as in patent WO09/087,391 and cauliflower mosaic virus 35S terminator sequence.

SEQ ID NO: 34:
atggccactactaaatcttttttaattttattttttatgatattagcaactactagttcaac
atgtgct

is an example of a nucleotide sequence that encodes the patatin signal peptide which is inserted at the 5′ end of the immunoglobulin heavy chain coding sequence in pC148.

The light chain with patatin signal peptide was placed under control of a plastocyanin promoter and terminator sequence as in patent WO01/25455.

> rituximab mature light chain (tobacco optimized)
sequence as in C148
(SEQ ID NO: 35)
cagattgtgctttctcagtctccagctattctttctgcttccccaggtgaaaaggttacaat
gacttgccgtgcttcttcttctgtgtcctacattcattggttccaacagaagccaggatctt
ctccaaagccatggatctacgctacttctaaccttgcttctggtgttccagttaggttttct
ggatctggatctggtacttcttactcccttactatttctagagtggaggctgaagatgctgc
tacttactactgccaacagtggacttctaatccaccaactttcggaggtggaactaagcttg
agatcaagaggactgttgctgctccatctgtgtttattttcccaccatctgatgagcaactt
aagtctggaactgcttctgttgtgtgccttctcaacaatttctacccaagggaagctaaggt
tcagtggaaagtggataatgctctccagtctggaaattctcaagagtctgtgactgagcagg
attctaaggattccacttactccctttcttctactcttactctctccaaggctgattatgag
aagcacaaggtttacgcttgcgaagttactcatcagggactttcttcaccagtgacaaagtc
cttcaaccgtggagagtgttga
(SEQ ID NO: 36)
QIVLSQSPAILSASPGEKVTMTCRASSSVSYIHWFQQKPGSSPKPWIYATSNLASGVPVRFS
GSGSGTSYSLTISRVEAEDAATYYCQQWTSNPPTFGGGTKLEIKRTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC*

The 5′ end of the immunoglobulin light chain coding sequence in pC148 is linked to a nucleotide sequence of SEQ ID NO: 37 that encodes the patatin signal peptide, wherein codon usage has been optimized for expression in tobacco.

(SEQ ID NO: 37)
atggccactactaagtccttccttatcctcttcttcatgatccttgctactacttcttctac
atgtgct

Both expression cassettes were cloned in the T-DNA part of pC100 described in Example 1 to generate pC148. pCambia-2300 (GenBank: AF234315.1; Hajdukiewicz et al., 1994. Plant. Mol. Biol. 25: 989-994) was amplified with primers PC201F (5′-AGAAGGCCTTCCGGGACGGCGTCAG-3′; SEQ ID NO: 8) and PC202R (5′-ATGGCGCGCCCCCCTCGGGATCA-3′; SEQ ID NO: 9) by PCR introducing unique StuI and AscI restriction endonuclease cleavage sites and subsequently ligated to the StuI/AscI fragment of pC148 comprising the rituximab expression cassette to generate pCambia-Rituximab.

The invention contemplates vectors according to any one of the preceding embodiments and as described above comprising, in the T-DNA region and operably linked to a plant regulatory element, a nucleotide sequence encoding the mature heavy chain of an immunoglobulin that binds human CD20 and exhibiting at least 90%, 92%, 94%, 96%, 98%, 99% or 99.5% sequence identity to SEQ ID NO: 32.

The invention also contemplates vectors according to any one of the preceding embodiments and as described above comprising, in the T-DNA region and operably linked to a plant regulatory element, a nucleotide sequence encoding the mature light chain of an immunoglobulin that binds human CD20 and exhibiting at least 90%, 92%, 94%, 96%, 98%, 99% or 99.5% sequence identity to SEQ ID NO: 35.

3.2 Infiltration of Nicotiana benthamiana Plants.

All binary vectors used in this study were introduced in Agrobacterium tumefaciens AGL1. Bacteria were grown in YEB-medium comprising 2 g/L Beef extract, 0.4 g/L Yeast extract, 2 g/L Bacto-Peptone, 2 g/L Sucrose, 0.1 g/L MgSO₄ and proper antibiotics for selection of the respective Agrobacterium strain and binary vector, in an erlenmeyer at 28° C. and 250 rpm on a rotary shaker up to an OD600>1.6. The culture was then diluted 1:100 in fresh LB Broth Miller medium containing 10 mM 2-(N-morpholino)ethanesulfonic acid (MES) and proper antibiotics and further grown at 28° C. and 250 rpm on a rotary shaker up to an OD600>2. After growth, bacteria were collected by centrifugation at 8000 g and 4° C. for 15 min. Pelleted bacteria were resuspended in infiltration solution containing 10 mM MgCl2 and 5 mM MES, final pH 5.6, and OD600=2. Four weeks old Nicotiana benthamiana plants were co-infiltrated with an Agrobacterium tumefaciens strain Agl1 containing the tomato bushy stunt virus (TBSV) p19 suppressor of gene silencing (Swiss-Prot P50625), and pC148 or pCambia-Rituximab at 1:1 ratio and final OD600 nm=0.3. The coding sequence for the TBSV p19 suppressor of gene silencing was under control of a double cauliflower mosaic virus 35S promoter and terminator sequence in pBin19 (Bevan MW (1984) Binary Agrobacterium vectors for plant transformation. Nucleic Acids Res. 12: 8711-8721). Vacuum infiltration was by immersion of the aerial part in a 10 L beaker filled with the bacterial inocula. Vacuum infiltration was performed in a glass bell jar (Schott-Duran Mobilex 300 mm) using a V-710 BUchi pump connected to a V-855 regulator and the pressure is decreased from atmospheric to 50 mbar absolute pressure in 3-4 minutes. Once reached, vacuum is kept for 1 min followed by a fast release in approximately 2 seconds. Artificial lighting (80-100 umol photon/cm²) is kept on during the whole infiltration process to ensure consistent light conditions. Following infiltration, plants were placed along with non-infiltrated control plants in the greenhouse until harvesting. Growth conditions such as fertilization, photoperiod and temperature are the same as used before infiltration. Water and fertilizer are administered to plants using a drip irrigation system.

### 3.3 Harvesting, Material Sampling and Analysis of Expression.

Six DAYS after infiltration, leaf material was collected in a heat-sealable pouch, sealed and placed between layers of dry-ice for at least 10 minutes. After harvesting, all leaf samples were stored at −80° C. until further processing. Harvested leaves were homogenized to a fine powder using a coffee-grinder on dry-ice and extracted in 3 vol/wt extraction buffer containing 50 mM Tris (pH 7.4), 150 mM NaCl, 0.1% Triton X-100, 4M Urea and 2 mM DTT. The expression of rituximab monoclonal antibody was quantified in the soluble extracts by ELISA. Plates (Immulon 2HB, Thermofisher) were coated overnight at 4° C. with a capture antibody (Goat anti-mouse IgG1 heavy chain specific Sigma, #M8770) at a concentration of 2.5 μg/mL. A standard curve (4-80 ng/mL) was prepared using Mouse IgG1 control protein (Bethyl, #MI10-102) in mock extract (prepared from leaf material infiltrated only with the p19 suppressor of gene silencing bacterial suspension). Soluble extracts were diluted 1:1000 in dilution buffer (50 mM Tris pH 7.4, 150 mM NaCl, 0.1% Triton X-100) and standards and samples were loaded in triplicate and incubated for 1 hour at 37° C. The antibody for detection was a peroxidase-conjugated goat anti-mouse IgG Fc-specific from Jackson ImmunoResearch (#115-035-205) which was used at a dilution of 1:40,000 and incubated for 1 hour at 37° C. Total soluble protein in the extracts was determined using the Coomassie-Plus Assay reagent from Pierce (#24236). Results of six experiments for each of the combinations, pC148 with p19 suppressor of gene silencing and pCambia-Rituximab with p19 suppressor of gene silencing, are presented in Table 4. The average expression of rituximab in Nicotiana benthamiana leaves was 136.30 mg/kg fresh weight (FW) leaves for pC148 compared to 122.60 mg/kg FW for pCambia-Rituximab (see Table 4).

TABLE 4
Yield of rituximab monoclonal antibody using the minimal
vector pC148 compared to a pCambia-derived expression
vectors in agroinfiltration of N. benthamiana plants

| | | Yield | | |
|---|---|---|---|---|
| | | mg/kg | Average | |
| Construct | Exp. | FW | mg/kg FW | SEM |
| pC148 | 1 | 138.85 | 136.30 | 3.09 |
| | 2 | 140.66 | | |
| | 3 | 142.68 | | |
| | 4 | 140.58 | | |
| | 5 | 122.61 | | |
| | 6 | 132.42 | | |
| pCambia- | 7 | 105.59 | 122.60 | 5.46 |
| Rituximab | 8 | 120.34 | | |
| | 9 | 118.12 | | |
| | 10 | 138.21 | | |
| | 11 | 138.98 | | |
| | 12 | 114.35 | | |

### Example 4

### Transient Expression of Influenza H5 Virus-Like Particle in Tobacco

### 4.1 Gene Constructs.

The gene coding for the HcPro suppressor of gene silencing of tobacco etch virus (TEV) isolate TEV7DA (GenBank: DQ986288.1) was cloned in the unique EcoRI site of pC100 to generate pC120. It was placed under the control of a double cauliflower mosaic virus 35S promoter, the 5′ untranslated region of TEV7DA and the nopaline synthase terminator sequence. Segment 4 of haemagglutinin H5N1 virus (GenBank: EF541394.1) comprising the coding sequence for mature haemagglutinin H5, was cloned under control of a minimal cauliflower mosaic virus 35 promoter, 5′- and 3′-untranslated regions of HT-CPMV and the nopaline synthase terminator sequence in the unique EcoRI site of pPMP1 (see Example 1) resulting in pC229.

4.2 Infiltration of Nicotiana tabacum Plants and Sample Preparation.

All gene constructs were introduced in Agrobacterium tumefaciens Agl1. Nicotiana tabacum plants were grown in the greenhouse in rockwool blocks with 20 h light, 26° C./20° C. day/night temperature and 70%/50% day/night relative humidity. Bacteria were grown as described in Example 3 to a final OD600 of 3.5. Agrobacterium cultures containing the pC229 gene construct and pC120 suppressor of gene silencing construct were mixed at a 3:1 ratio and diluted to a OD600=0.8 in infiltration solution containing 10 mM MgCl2 and 5 mM MES, pH5.6. Plants were infiltrated by immersion of the aerial part in a 10 L beaker filled with the bacterial inoculum. Vacuum infiltration was performed in a vacuum chamber by decreasing the pressure to 900 mbar below atmospheric pressure within 15 s, a 60 s holding time followed by a fast release for approximately 2 s. Following infiltration, plants were placed back in the greenhouse and incubated under the same environmental conditions as before infiltration. Leaves of infiltrated plants were collected from 10 plants at 5 days post infiltration and homogenized using a screw press (Green Star Corrupad, GS 1000, Korea Co.). Sodium metabisulphite was added to 10 mM final concentration to avoid sample oxidation. The pH of the extract was adjusted to pH 5.3 and subsequently incubated at room temperature for 20-30 min without stirring. Celpure P300 (10%) was then added to the extract and mixed for 1 minute. The solution was filtrated through a Whatman filter paper pre-coated with Celpure P300 (10% Celpure P300 slurry in 10 mM sodium metabisulphite). For ultracentrifugation, sucrose cushions of 3 ml were prepared in ultracentrifuge tubes by carefully layering. Three different cushions were prepared as follows: 1) 3 mL of 80% sucrose; 2) 1.5 ml each of 60 and 45% sucrose; and 3) 1 mL each of 60, 45 and 35% sucrose. Clarified and filtered extract samples (up to 13 mL) were gently placed on top of the sucrose gradients and subjected to ultracentrifugation. Centrifugation was in a swinging bucket type rotor (Sorvall Surespin 630; Kendro) at 24,000 rpm for 1 hour at 4° C. (135,000 RCFmax). Sucrose concentrated samples were pre-filtered using a 0.45 Nm filter and subjected to size exclusion chromatography (SEC) under isocratic conditions on an automated AKTA chromatography system. Running buffer was TBS, pH 7.5 and sample size was 4 mL under a flow rate of 1 mL/min on a HiLoad 16/60 Superdex 200 column (GE Healthcare, 17-1069-01). Fractions containing purified H5 VLP as apparent from SDS-PAGE and Western blotting (see FIGS. 3 & 4) were pooled and concentrated to about 0.3 mg/mL using a 30 kDa cut-off Centricon ultrafiltration membrane device (Millipore) and further analysed.

### 4.3 Gel Electrophoresis and Western Blotting.

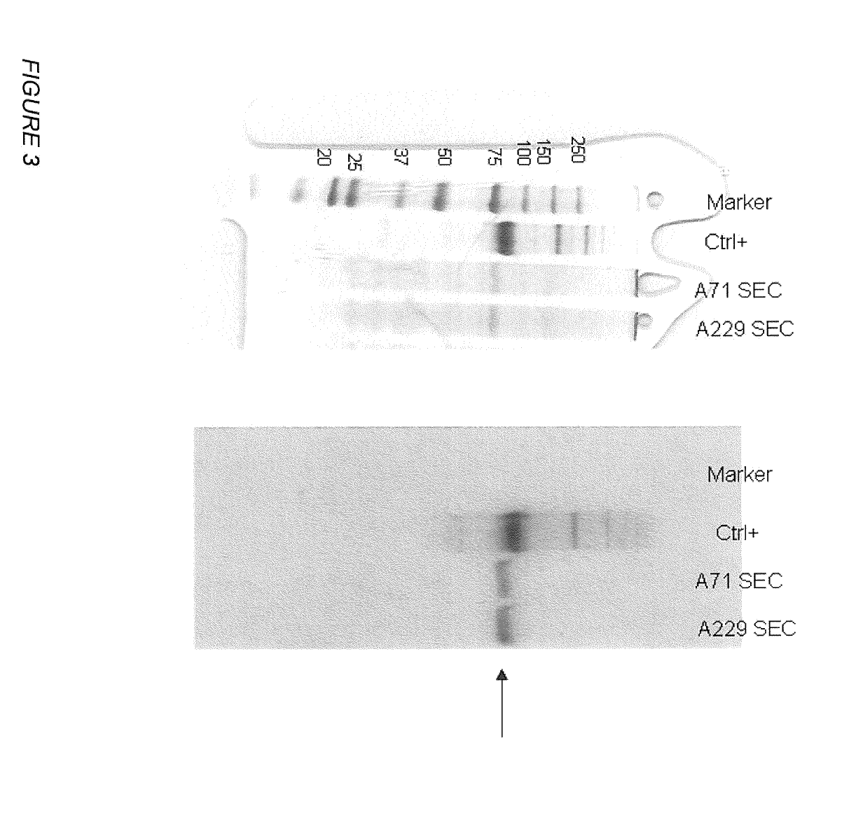

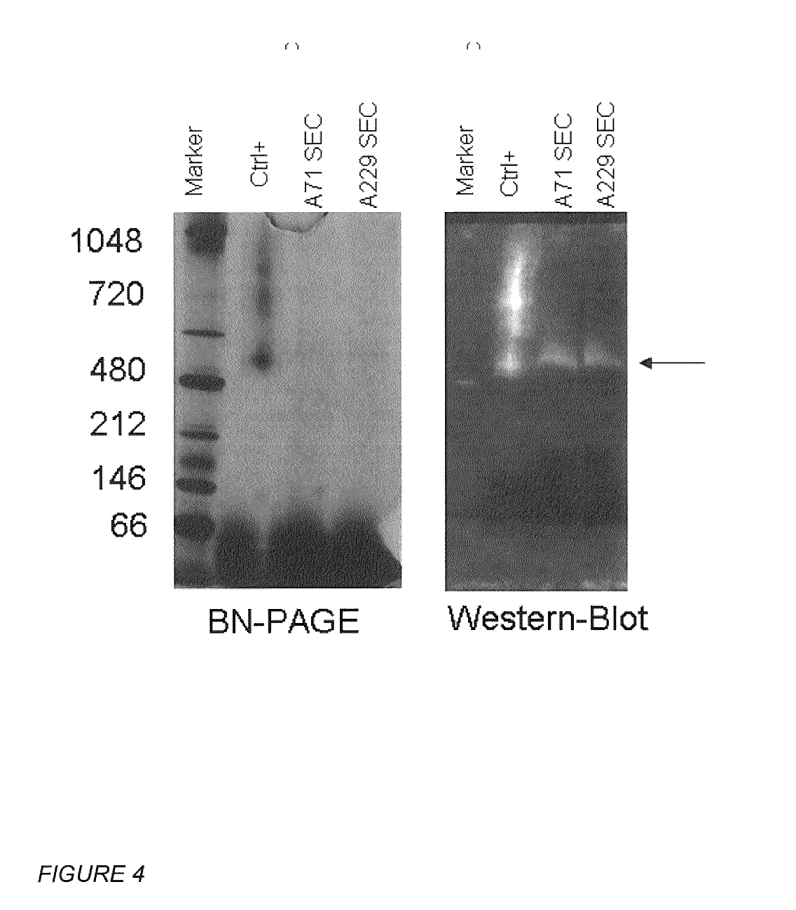

Samples of pooled fractions were subjected to SDS-PAGE (FIG. 3A), western blotting (FIGS. 3B & 4B) and Blue Native-PAGE (FIG. 4A) using standard techniques. SDS-PAGE was on a 4-12% SDS-PAGE gel. As a control (Ctrl+) commercially available recombinant H5 (Immune-tech, cat. #IT-003-0052p) was used. After separation, proteins were stained with Imperial M protein stain (Pierce #24615). For Western blotting, the primary antibody was a rabbit anti-HA antibody (H5N1 VN1203/04 # IT 003-005V). For detection, an HRP-labelled affiniPure goat-anti-rabbit IgG FC-fragment was used (Jackson #111-035-046). Detection was done by chemiluminescence using an Immuno-star HRP Chemiluminescent Kit (BIO-RAD Laboratory, 170-5040). Results were captured using Chimio-Capt 3000 and are shown in FIG. 3B. FIGS. 3A and B clearly show the presence of H5 in extracts of plants infiltrated with the pC229 gene construct of similar size as commercial recombinant H5. Native-PAGE was performed on 4-16% Bis-Tris PAGE gels (Novex). For loading, samples were treated with Digitonin in Native-PAGE sample buffer (Novex) and incubated for 1 h at 4° C. Subsequently, Native-PAGE G-250 sample additive (Novex) was added to a final concentration of 0.5% and samples were loaded and run on a 4-16% Bis-Tris PAGE gel. Gels were run at 4° C. at 150V constant for the first 60 min. Subsequently, voltage was increased to 250V for another 30 min and gels were stained with Imperial M protein stain. Results of native-PAGE are represented in FIG. 4A. Results of Western blotting are shown in FIG. 4B and clearly show the successful expression of H5 following transient expression in tobacco.

### 4.4 Haemagglutination.

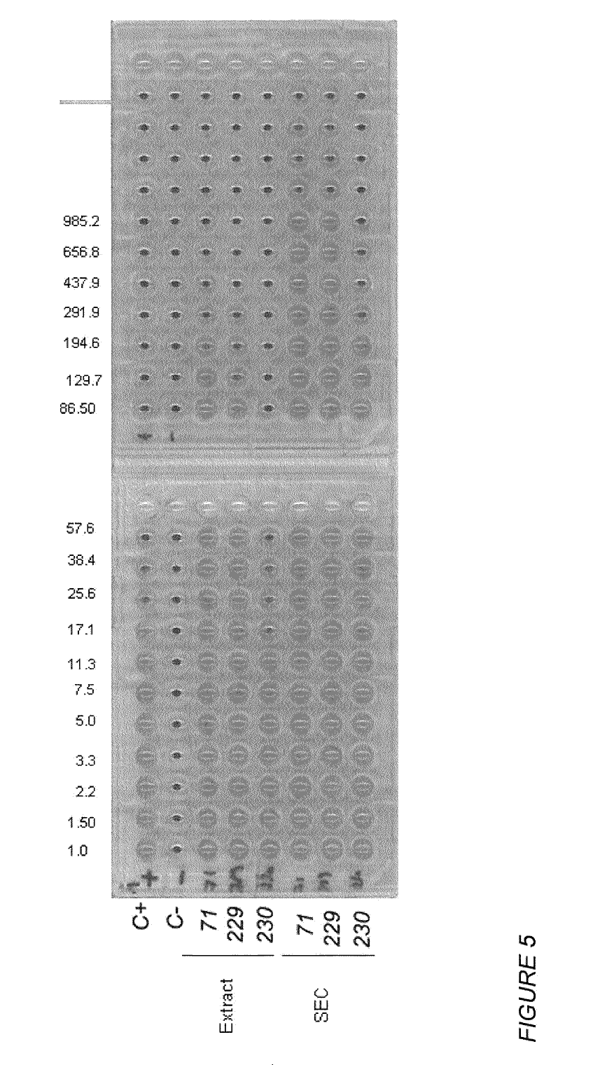

Natural trimeric H5 protein has the ability to bind to the monosaccharide sialic acid, which is present on the surface of erythrocytes (red blood cells). This property called hemagglutination is the basis of a rapid assay and was used here to determine the biological activity of the recombinant protein. Haemagglutinating activity of tobacco produced H5 was measured by incubating 1.5-fold serial dilutions of the plant extract as well as extract purified by size-exclusion chromatography (SEC) in a 96-well plate with a specific amount of red blood cells (FIG. 5). Red blood cells not bound to HA sink to the bottom of a well and form a precipitate. It is important to note that only HA correctly assembled as homo-trimer will bind erythrocytes. FIG. 5 shows haemagglutinating activity is observed in extracts of tobacco plants infiltrated with the pC229 gene construct, as well as in size exclusion chromatography enriched fractions of pC229.

### Example 5

### Determination of Backbone Integration in Single Copy Transgenic Plants

Backbone-free single copy transgenic plants are highly desired from a regulatory perspective and are less prone to silencing of the transgene. To determine the presence of pC100 vector backbone polynucleotide sequences in the transgenic plants with a single copy T-DNA integration, as obtained following transformation of tobacco with pC100 as described in Example 2, a PCR was performed on all single copy pC100 transgenic tobacco plants using specific primers to amplify certain pC100 vector polynucleotide sequences.

### 5.1 Primer Sequences and PCR Amplification.

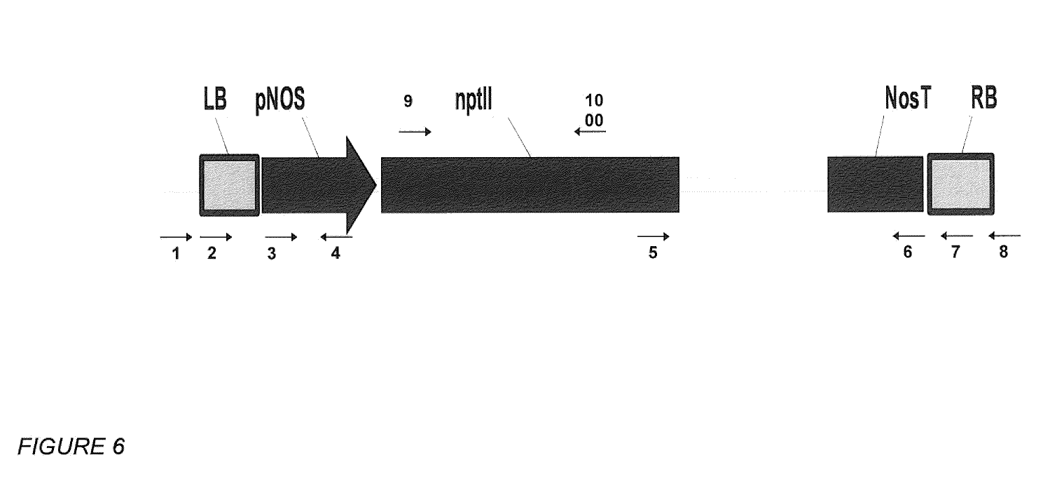

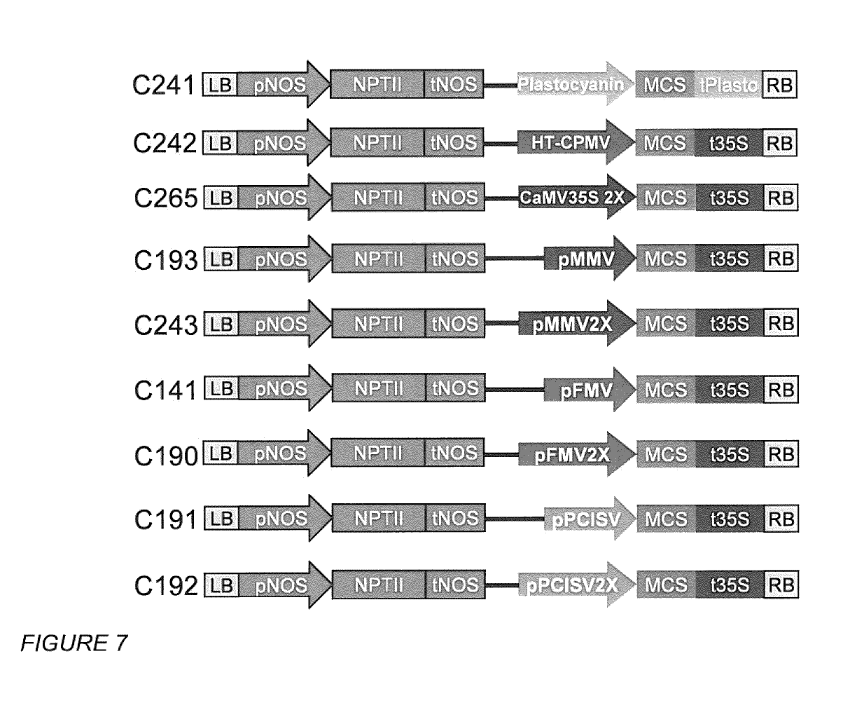

Genomic DNA of 73 single copy transgenic tobacco plants transformed with pC100 was isolated using standard methods. Using 10 different primers as listed in Table 5 and selected from the list of SEQ ID NO:10 to SEQ ID NO:19, 7 fragments could be amplified based on the presence of pC100 vector backbone or T-DNA sequences in each of the transgenic plants. The location of the primer sequences along the pC100 vector is schematically represented in FIG. 6. Primer 1 (SEQ ID NO:10: 5′-GAGCTGTTGGCTGGCTGG-3′) is part of the backbone vector sequence and is located upstream of the T-DNA left border sequence of pC100 from −18 to −1 relative to the T-DNA left border nick site.

Primer 2 (SEQ ID NO:11: 5′-GGCAGGATATATTGTGGTGTAAAC-3′) is part of the T-DNA left border sequence of pC100 and is located from basepair +2 to +25 relative to the left T-DNA border nick site.

Primer 3 (SEQ ID NO:12: 5′-GACCCCCGCCGATGAC-3′) is part of the nopaline synthase promoter controlling the expression of the NPTII gene and is located downstream of the T-DNA left border from basepair +122 to +137 relative to the T-DNA left border nick site of pC100.

Primer 4 (SEQ ID NO:13: 5′-CGCAATAATGGTTTCTGACGTA-3′) is part of the nopaline synthase promoter and is located down of the T-DNA left border from basepair +264 to +232 on the bottom strand relative to the T-DNA left border nick site of pC100.

Primer 5 (SEQ ID NO:14: 5′-GTGATATTGCTGAAGAGCTTGG-3′) is part of the carboxy terminus of the NPTII gene and is located upstream of the T-DNA right border from basepair −870 to −848 on the upper strand relative to the T-DNA right border nick site of pC100.

Primer 6 (SEQ ID NO: 15: 5′-TTGCGCGCTATATTTTGTTTTC-3′) is part of the nopaline synthase terminator sequence bottom strand and is located from basepair −171 to −151 relative to the T-DNA right border nick site of pC100.

Primer 7 (SEQ ID NO: 16: 5′-TAAACGCTCTTTTCTCTTAGGTTTAC-3′) covers the T-DNA right border sequence from −26 to −1 on the bottom strand and relative to the T-DNA right border nick site of pC100.

Primer 8 (SEQ ID NO: 17: 5′-AGGCGCTCGGTCTTGG-3′) is part of the backbone vector bottom strand sequence downstream of the T-DNA right border and located from basepair +87 to +102 relative to the T-DNA right border nick site of pC100.

Primers 9 (SEQ ID NO: 18: 5′-GCGTTGGCTACCCGTGATAT-3′) and 10 (SEQ ID NO: 19: 5′-ACATGCTTAACGTAATTCAAGAG-3′) are T-DNA internal primers located in the NPTII gene pC100.

Primer pair 1 & 4 generates a 272 bp fragment containing part of the pC100 backbone sequence upstream of the left border sequence up to the nopaline synthase promoter, Primer pair 2 & 4 generates a 253 bp fragment containing the left border sequence up to the nopaline synthase promoter. Primer pair 3 & 4 generates a 133 bp fragment containing the nopaline synthase promoter and coding sequence located on the T-DNA of pC100. Primer pair 5 & 6 generates a 720 bp fragment containing the nopaline synthase coding and terminator sequence located on the T-DNA of pC100. Primer pair 5 & 7 generates a 870 bp fragment containing the nopaline synthase coding and terminator sequence and T-DNA right border sequence of pC100. Primer pair 5 & 8 generates a 972 bp fragment containing the nopaline synthase coding and terminator sequence as well as T-DNA right border and pC100 backbone vector sequence downstream of the right T-DNA border. Primer pair 9 & 10 generates a 626 bp internal NPTII coding sequence fragment. Plant genomic DNA of all 73 single copy plants was amplified by PCR using Mastercycler gradient machine (Eppendorf). Reactions were performed in 20 μl including 10 μl of GoTaq green Master Mix (2×) (Promega), 7.5 μl of water, 1 μl of DNA, 0.5 μl of MgCl₂ (25 mM) and 0.5 μl of each of the primers (10 μM) as listed and according to Table 5. Thermocycler conditions were set-up as indicated by the supplier using 60° C. as annealing temperature. PCR products were loaded on a 1% agarose gel and migrated for 1 hour at 80V. Sizes of PCR products were analyzed using the gel documentation system ChemiSmart (Fisher Biotec) and a sample was determined positive for a given primer set if the resulting PCR fragment matched the expected fragment size as indicated for the given primer pair.

### 5.2 Results.

Results are summarized in Table 5. All single copy transgenic plants (73/73) were positive for the internal fragment as amplified by primers 9 & 10. None of the 73 plants contained vector backbone pC100 sequence downstream of the T-DNA right border nick sequence and 10 out of 73 (14%) had the T-DNA right border sequence integrated. Only 3 out of 73 plants missed some part of the nopaline synthase terminator sequence directly flanking the T-DNA right border sequence and the remaining 70 (96%) were positive for primer pair 5 & 6 indicating correct integration at the right side of the T-DNA. On the left side 18 out of 73 plants (25%) had some vector backbone pC100 sequence upstream of the T-DNA left border. 18 out of 73 plants (25%) also had the left border sequence integrated which according to the experimental design and FIG. 6 schematic drawing of primer location, should be the same 18 plants as those that had left border vector backbone sequences. 63 out of 73 plants (86%) were positive for the nopaline synthase coding and promoter sequence indicating correct integration at the left side of the T-DNA.

TABLE 5
PCR results of 73 single copy pC100 transformed transgenic
tobacco plants using various pairs of primers amplifying
downstream right and upstream left border vector backbone
and T-DNA sequences, or T-DNA internal sequences only.

| | Expected | Positive plants |
|---|---|---|
| Primer pair | size amplicon | (%) |
| 1 & 4 | 272 bp | 18/73 |
| | | (25%) |
| 2 & 4 | 253 bp | 18/73 |
| | | (25%) |
| 3 & 4 | 133 bp | 63/73 |
| | | (86%) |
| 5 & 6 | 720 bp | 70/73 |
| | | (96%) |
| 5 & 7 | 870 bp | 10/73 |
| | | (14%) |
| 5 & 8 | 972 bp | 0/73 |
| | | (0) |
| 9 & 10 | 626 bp | 73/73 |
| | | (100%) |

### Example 6

### Comparisons of Regulatory Elements for Transient Expression in Nicotiana tabacum

### 6.1 Promoter and Regulatory Region:

A number of candidate promoter regions and regulatory sequences that allow enhanced expression at transcriptional or translational level were compared. A library of vectors derived from the minimal vector pC100 with these expression cassettes inserted in the T-DNA region were created. These “ready-to-clone” vectors allow easy insertion of different genes encoding proteins of interest. The experiment described below aimed at rapidly evaluating for two different proteins, H5 from influenza and mature human glucocerebrosidase (GBA).

TABLE 6
Vectors generated from pC100 with or without
plant selectable marker

| | | pC100- | | pC100-derived |
|---|---|---|---|---|
| | pC100- | derived | pC100-derived | vectors with |
| | derived | vectors with | vector without | H5 insert and |
| Promoter | vectors | H5 insert | nptII sequence | without nptII |
| pMMV | pC193 | pC264 | pC277 | pC275 |
| pMMV 2x | pC243 | pC259 | pC278 | pC270 |
| pFMV | pC141 | pC260 | pC279 | pC271 |
| pFMV 2x | pC190 | pC261 | pC280 | pC272 |
| pPCISV | pC192 | pC262 | pC281 | pC273 |
| pPCISV 2x | pC265 | pC263 | pC282 | pC274 |
| CaMV35S 2x | pC265 | pC266 | | pC276 |

### 6.2 Glucocerebrosidase (GBA):

The mature human glucocerebrosidase (GBA) protein sequence used in all GBA vectors corresponds to the sequence of accession NP_{—}000148.2. The DNA sequence set forth in SEQ ID NO: 38 was codon-optimized for tobacco and chemically synthesized. Thus. the invention contemplates vectors according to any one of the preceding embodiments as described above comprising, in the T-DNA region and operably linked to a plant regulatory element, a nucleotide sequence encoding a mature human glucocerebrosidase and exhibiting at least 90%, 92%, 94%, 96%, 98%, 99% or 99.5% sequence identity to SEQ ID NO: 38.

Three GBA sequences were synthesized encoding three forms of the protein:
Secreted GBA: the N-terminal secretion signal peptide from Solanum tuberosum Patatin A (GeneBank accession number CAA25592.1) was fused to the GBA mature sequence to target the protein through the secretion pathway to the apoplast. The resulting mature product is expected to display primary amino acid sequence comparable to the native protein.
Endoplasmic reticulum (ER)-retained GBA: the N-terminal Patatin A peptide was fused to the GBA mature sequence to target the protein through the secretion pathway and in addition a KDEL peptide, i.e. plant-specific ER retention signal was fused to the C-terminus. The resulting mature product is expected to possess KDEL as extra amino acids at the C-terminus.
Vacuolar GBA: the N-terminal Patatin A peptide was fused to the GBA mature sequence to target the protein through the secretion pathway and in addition a tobacco Chitinase A vacuolar targeting signal (Shaaltiel Y. et al. 2007) was fused to the C-terminus. The resulting mature product is expected to possess 7 extra amino acids at the C-terminus corresponding to the vacuolar targeting signal peptide.

GBA sequences were introduced into three pC100-derived vectors comprising a plastocyanin vector, a double MMV promoter (pMMV 2×) and the HT-CPMV system.

> mature GBA (tobacco optimized) sequence as delivered
by synthesis
(SEQ ID NO: 38)
gctagaccatgcattcctaagtctttcggttactcttctgttgtgtgcgtgtgcaatgctac
ttactgcgattctttcgatcctcctacttttcctgctcttggtactttttctaggtacgagt
ctaccaggtctggtagaagaatggaactttctatgggtcctatccaggctaatcatactggt
actggtctgcttcttactcttcaacctgagcagaagttccaaaaggttaagggttttggtgg
tgctatgactgatgctgctgctcttaatattctggctctttctcctcctgctcaaaacttgc
tgctgaagtcttacttcagcgaagagggtatcggttacaacattattagggtgccaatggct
tcctgcgatttctctattaggacttatacctacgctgatacccctgatgatttccagcttca
caactttagcctgcctgaagaggataccaagctgaagattcctcttattcatagggctctgc
agcttgctcaaagacctgtttctcttttggcttctccttggacttctcctacttggcttaag
actaatggtgctgtgaacggtaagggttctcttaagggtcaacctggtgatatctaccatca
aacttgggctagatacttcgtgaagttccttgatgcttacgctgagcataagttgcagtttt
gggctgttactgctgagaatgagccttctgctggtcttttgtctggttatccttttcagtgc
cttggtttcactcctgaacatcagagggatttcattgctagagatttgggtcctacccttgc
taattctactcatcataacgtgaggctgctgatgcttgatgatcagagacttcttttgcctc
actgggctaaggttgtgcttactgatcctgaagctgctaagtacgttcacggtattgctgtt
cactggtacttggattttctggctcctgctaaggctactcttggtgaaactcataggctttt
ccctaacaccatgctttttgcttcagaggcttgcgttggttctaagttttgggaacagtctg
tgagacttggatcttgggatagaggtatgcagtacagccactctattattaccaacctgctg
taccatgtggtgggttggactgattggaatcttgctcttaatcctgagggtggtcctaattg
ggttaggaatttcgtggatagccctatcatcgtggatattaccaaggataccttctacaagc
agcctatgttctaccatcttggtcacttcagcaagttcattccagaaggttctcagagggtt
ggacttgttgcttctcaaaagaacgatcttgatgctgtggctcttatgcaccctgatggttc
tgctgttgttgttgtgcttaacaggtctagcaaggatgtgcctctgactatcaaagatcctg
ctgttggtttcttagagaccatttctcctggttactctattcacacctacctttggcgtcga
caa
(SEQ ID NO: 39)
ARPCIPKSFGYSSVVCVCNATYCDSFDPPTFPALGTFSRYESTRSGRRMELSMGPIQANHTG
TGLLLTLQPEQKFQKVKGFGGAMTDAAALNILALSPPAQNLLLKSYFSEEGIGYNIIRVPMA
SCDFSIRTYTYADTPDDFQLHNFSLPEEDTKLKIPLIHRALQLAQRPVSLLASPWTSPTWLK
TNGAVNGKGSLKGQPGDIYHQTWARYFVKFLDAYAEHKLQFWAVTAENEPSAGLLSGYPFQC
LGFTPEHQRDFIARDLGPTLANSTHHNVRLLMLDDQRLLLPHWAKVVLTDPEAAKYVHGIAV
HWYLDFLAPAKATLGETHRLFPNTMLFASEACVGSKFWEQSVRLGSWDRGMQYSHSIITNLL
YHVVGWTDWNLALNPEGGPNWVRNFVDSPIIVDITKDTFYKQPMFYHLGHFSKFIPEGSQRV
GLVASQKNDLDAVALMHPDGSAVVVVLNRSSKDVPLTIKDPAVGFLETISPGYSIHTYLWRR
Q
> patatin tobacco optimized sequence (slightly modified for
cloning reasons) as delivered by synthesis in front of GBA
(SEQ ID NO: 40)
Atggctactactaagtctttcctgatcctgttcttcatgattcttgctactacctcgagcac
gtgtgct

TABLE 7
Vectors comprising a sequence encoding a form of GBA

| C100-derived | Expression | | |
|---|---|---|---|
| vector | Cassette | Product | Vector code |
| C241 | Plastocyanin | Secreted GBA | C248 |
| | | ER-retained | C249 |
| | | GBA | |
| | | Vacuolar GBA | C251 |
| C242 | HT-CPMV | Secreted GBA | C252 |
| | | ER-retained | C253 |
| | | GBA | |
| | | Vacuolar GBA | C254 |
| C243 | pMMV 2x | Secreted GBA | C255 |
| | | ER-retained | C256 |
| | | GBA | |
| | | Vacuolar GBA | C257 |

6.3 Growing and Infiltration of N tabacum:

N. tabacum plants were germinated and grown in the greenhouse with 20 h light, 26° C./20° C. day/night temperature and 70%/50% day/night relative humidity. Artificial lighting is turned automatically on between 02h00 to 22h00 when natural light is under 200 W/m2 (20 hours light) with a 15,000 or 20,000 Lux lighting system giving a PAR of about 100 μmol/m²/s. Plants were germinated in floating trays and at the end of week 3 were grown until they reached the desired developmental stage. All constructs were introduced into Agrobacterium strain AGL1 by infiltration. Following infiltration, plants were incubated on greenhouse until harvesting and conditions such as fertilization, photoperiod and temperature were the same as used pre-infiltration. Leaf material was collected at 5 days post infiltration (dpi). Stems, petioles and non-infiltrated biomass at the apex of the plants were not harvested. The leaf material was then homogenized to a fine powder using a coffee-grinder and dry-ice.

### 6.4 Leaf Extraction:

Aliquots of frozen leaf powder were extracted in H5 extraction buffer (1×PBS, 1% Tween-20 (v/v), 4M Urea) at a ratio of 1 g frozen powder to 3 mL buffer, by two steps of vortexing, followed by centrifugation at 20,000 g for 15 min. Soluble extracts were mixed at a 3:1 ratio (v/v) with NuPAGE LDS 4× Sample Buffer containing 50 mM DTT and heated to 95° C. for 5 min before loading on a 4-12% Bis-Tris NuPAGE gel (10 uL per well). Western blotting was performed by standard techniques. Primary antibody Rabbit anti-HA (H5N1 (VN1203/04) IgG was diluted 1:1,000 (v:v). Secondary antibody HRP-conjugated goat anti-rabbit (Jackson; 11-035-046) was diluted 1:10,000. Preparation of crude extracts of agro-infiltrated tobacco and enzyme-linked immunosorbent assay (ELISA) for the quantification of H5 protein was performed by standard techniques.

### 6.5 Determination and Comparision of Protein Expression:

A direct comparison of H5 expression in N. tabacum obtained with pMMV 2× (pC259) and HT-CPMV (pC71) was performed. Agl1 inoculum at OD600 of 0.55 were used. A minimal vector comprising Hc-Pro as a suppressor of silencing in a separate construct (pC288) was co-infiltrated at a ratio of construct of interest (COI): suppressor of silencing (SoS) of 3:1. Sampling from 12 plants per branch of the experiment: A259+A228 (pMMV 2×) produced approximately 65 mgH5/kg frozen material and A71+A228 produced approximately 55 mgH5/kg frozen material. The results confirm that pMMV 2× could represent a good alternative to the HT-CPMV expression cassette for transient expression of recombinant proteins in N. tabacum.

6.6 Infiltration of N tabacum with Constructs Encoding H5:

Constructs encoding H5 under the control of the different single- and/or double-enhanced constitutive promoters isolated from Caulimoviruses were generated using the pC100-derived vectors and transformed into A. tumefaciens strain AGL1.

N. tabacum plants were co-infiltrated (OD600 of 0.5 and ratio of COI:SoS of 1:1) with A228 (AGL1 carrying C228 construct encoding the HcPro SoS in minimal vector) and AGO carrying one of the constructs encoding H5 under each of the expression cassettes: HT-CPMV (A71), pMMV 2× (A259), pFMV 1× (A260), pFMV 2× (A261), pPCISV 1× (A262), pPCISV 2× (A263), pMMV 1× (A264) or pCaMV 35S 2× (A266). All combinations were compared over 3 infiltration events. Triplicate pools of 4 agro-infiltrated plants were harvested at 5 days post infiltration and the H5 concentration of each was determined by ELISA.

### 6.7 Determination of H5 Expression and Comparision:

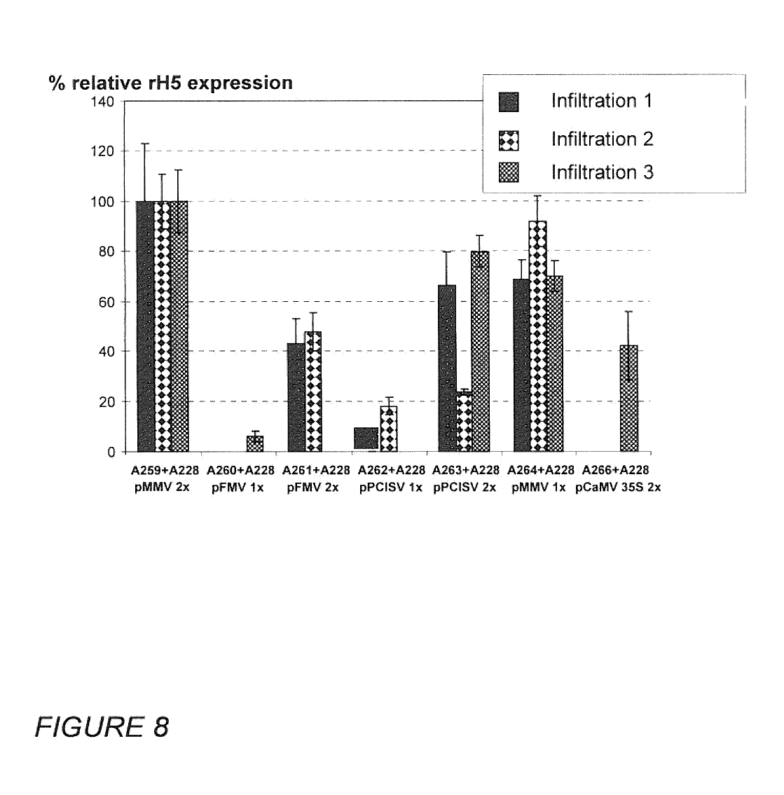

To allow comparison over the 3 infiltrations, H5 concentration in the biomass obtained with the different expression cassetttes were expressed as a percentage of the concentration obtained with pMMV 2×, which displayed the highest level of protein accumulation in all thre infiltration events (FIG. 8). Very low H5 expression levels were obtained using single-enhanced FMV (A260) and PCISV (A262) promoters. Insertion of an additional enhancer element to the PCISV and FMV promoters increased their strength by several folds. Double-enhanced PCISV promoter (A263) led to H5 accumulation comparable to that obtained with the single-enhanced MMV promoter (A264) also approaching (70-90%) H5 concentrations obtained with the double-enhanced MMV promoter. However, H5 expression remained 40-50% lower with the double-enhanced FMV promoter (A261). H5 protein accumulation obtained with CaMV 35S 2× promoter was only 40% of that obtained with the double-enhanced MMV promoter.

### 6.7.1 Western Blot Analysis of GBA:

Aliquots of frozen leaf powder were extracted in GBA extraction buffer: PBS 1×Tween-80 0.15% (v/v), Sodium taurocholate 0.15% (w/v), 5 mM DTT, at a ratio of 1 g frozen powder to 3 mL buffer, by two steps of vortexing, followed by centrifugation at 20,000 g for 15 min. Soluble extracts were mixed at a 3:1 ratio (v/v) with NuPAGE LDS 4× Sample Buffer containing 50 mM DTT and heated to 95° C. for 5 min before loading on a 4-12% Bis-Tris NuPAGE gel (10 uL or 7 uL per well in 10 or 15-well respectively). Western blotting was performed according to standard techniques. Primary antibody: Sigma G4046, anti-GBA peptide raised in rabbit, at a dilution of 1:2,000 and secondary antibody: Jackson 11-035-046, anti-rabbit HRP-conjugated, at a dilution of 1:5,000.

### 6.7.2 GBA Quantification by Enzymatic Assay.

Quantification of recombinant GBA in crude extracts of infiltrated N. tabacum was performed by enzymatic assay relative to a standard curve established with the reference protein Cerezyme®.

### 6.8 Results:

With respect to relative expression of GBA with the different promoters, Western blot analysis indicated that the double enhanced pMMV 2× promoter was yielding the strongest GBA protein expression followed by the HT-CPMV translator enhancer cassette (estimated 2-4-fold lower according to the serial dilutions) and the weakest expression was obtained with the Plastocyanin cassette (estimated 8-fold lower).

Using Cerezyme® (Genzyme Corp.) as a standard, Western blots and Coomassie-stained SDS-PAGE gels showed that the intensity of the band from known amounts of Cerezyme spiked in plant extract with the intensity of the band corresponding to tobacco produced GBA protein in extracts of infiltrated leaf. For extracts A255, A256 and A257 prepared from leaf material infiltrated with (pMMV 2× expression cassette), a band corresponding to GBA was visible on Coomassie-stained gels. Analysis by western blot of the accumulation overtime of the vacuolar GBA protein under control of the 3 different expression cassettes indicated that GBA concentration was lower after 3 days post infiltration (dpi) and appeared then relatively stable between day 4 and day 7 post-infiltration.

An enzymatic assay for the quantification of GBA concentration in crude extracts was developed and it is based on the hydrolysis by GBA of a synthetic substrate 4-MUG and release of a fluorescent product 4-MU. Plant extracts containing GBA are incubated in the presence of an excess of substrate and the enzymatic reaction is considered at steady state (constant rate of product accumulation). A standard curve is established by measuring the fluorescence (linearly related to the amount of 4-MU produced by the reaction) after the assay is run with serial dilutions of known concentrations of the reference protein Cerezyme. It is important to keep in mind that quantification of the concentration of recombinant GBA protein in crude extracts using this enzymatic assay relies on the verified assumption that Cerezyme and the tobacco-produced hGBA have a similar Km for the substrate across all plant material and constructs. Comparability of Km was demonstrated for the 3 GBA proteins (targeting to the vacuole, secretion pathway or ER-retained) transiently expressed under the double enhanced pMMV promoter. The following Km values were obtained in one single experiment: C255: 1.4 mM; C256: 1.3 mM, C257: 1.2 mM, Cerezyme: 1.3 mM (refer to Products Candidates Development-GBA-PACK for more details).

The results obtained by enzymatic assay were in agreement with results obtained from Coomassie/Western blot. They confirm that under the conditions used, the double enhanced pMMV promoter is yielding the strongest GBA protein expression followed by the HT-CPMV translator enhancer cassette and the weakest expression is obtained with the Plastocyanin promoter. In addition, the hGBA protein targeted to the vacuole appears to accumulate to higher concentrations than either the ER-retained or secreted form.

TABLE 8
GBA produced in N. tabacum by various plant
regulatory elements

| | | Quantification |
|---|---|---|
| | | by Enzymatic |
| | | assay |
| | | (mg GBA/kg |
| Extracts by | | frozen leaf |
| GBA Construct | Expression cassette | biomass) |
| A248 | Plastocyanin - Secreted GBA | 5-10 |
| A249 | Plastocyanin - ER-retained GBA | |
| A251 | Plastocyanin - vacuolar GBA | |
| A252 | 35S HT-CPMV - Secreted GBA | could not be |
| | | determined |
| A253 | 35S HT-CPMV - ER-retained GBA | could not be |
| | | determined |
| A254 | 35S HT-CPMV - vacuolar GBA | 20-35 |
| A255 | 2x pMMV - Secreted GBA | approx. 20 |
| A256 | 2x pMMV - ER-retained GBA | 20-35 |
| A257 | 2x pMMV - vacuolar GBA | 80-120 |

Among the seven Caulimovirus promoters tested, the double-enhanced FLt promoter from Mirabilis Mosaic Virus (pMMV 2×) was the strongest promoter for H5 expression. This promoter may represent a possible alternative to HT-CPMV system for transient H5 production in N. tabacum yielding comparable H5 accumulation at bench scale. Comparable TurboGFP expression was also obtained with both the pMMV 2× and the HT-CPMV expression cassettes.

For expression of GBA, three promoters were compared, the pMMV 2×, HT-CPMV and a plant promoter plastocyanin. In the conditions used for the experiments, the highest protein accumulation (approx. 100 mg/kg leaf biomass) was obtained with the double enhanced pMMV promoter followed by the HT-CPMV translator enhancer cassette (at least 2× less) and the weakest expression was obtained with the plastocyanin promoter (at least 4× less).

In the description and examples, reference is made to the following sequences that are represented in the sequence listing:

SEQ ID NO: 1: nucleotide sequence of vector pPMP1
ctactagtcccctagtacattaaaaacgtccgcaatgtgttattaagttgtctaagcgtcaa
tttgtttacaccacaatatatcctgccaccagccagccaacagctccccgaccggcagctcg
gcacaaaatcaccactcgatacaggcagcccatcaggccttgacggccttccttcaattcgc
cctatagtgagtcgtattacgtcgcgctcactggccgtcgttttacaacgtcgtgactggga
aaaccctggcgttacccaacttaatcgccttgcagcacatccccctttcgccagctggcgta
atagcgaagaggcccgcaccgaaacgcccttcccaacagttgcgcagcctgaatggcgaatg
ggagcgccctgtagcggccactcaaccctatctcggtctattcttttgatttataagggatt
ttgccgatttcggcctattggttaaaaaatgagctgatttaacaaaaatttaacgcgaattt
taacaaaatattaacgcttacaatttaggtggcacttttcggggaaatgtgcgcggaacccc
tatttgtttatttttctaaatacattcaaatatgtatccgctcatgagacaataaccctgat
aaatgcttcaataatattgaaaaaggaagagtatgattgaacaggatggcctgcatgcgggt
agcccggcagcgtgggtggaacgtctgtttggctatgattgggcgcagcagaccattggctg
ctctgatgcggcggtgtttcgtctgagcgcgcagggtcgtccggtgctgtttgtgaaaaccg
atctgagcggtgcgctgaacgagctgcaggatgaagcggcgcgtctgagctggctggccacc
accggtgttccgtgtgcggcggtgctggatgtggtgaccgaagcgggccgtgattggctgct
gctgggcgaagtgccgggtcaggatctgctgtctagccatctggcgccggcagaaaaagtga
gcattatggcggatgccatgcgtcgtctgcataccctggacccggcgacctgtccgtttgat
catcaggcgaaacatcgtattgaacgtgcgcgtacccgtatggaagcgggcctggtggatca
ggatgatctggatgaagaacatcagggcctggcaccggcagagctgtttgcgcgtctgaaag
cgagcatgccggatggcgaagatctggtggtgacccatggtgatgcgtgcctgccgaacatt
atggtggaaaatggccgttttagcggctttattgattgcggccgtctgggcgtggcggatcg
ttatcaggatattgcgctggccacccgtgatattgcggaagaactgggcggcgaatgggcgg
atcgttttctggtgctgtatggcattgcggcaccggatagccagcgtattgcgttttatcgt
ctgctggatgaatttttctaataactgtcagaccaagtttactcatatatactttagattga
tttaaaacttcatttttaatttaaaaggatctaggtgaagatcctttttgataatctcatga
ccaaaatcccttaacgtgagttttcgttccactgagcgtcagaccccgtagaaaagatcaaa
ggatcttcttgagatcctttttttctgcgcgtaatctgctgcttgcaaacaaaaaaaccacc
gctaccagcggtggtttgtttgccggatcaagagctaccaactctttttccgaaggtaactg
gcttcagcagagcgcagataccaaatactgttcttctagtgtagccgtagttaggccaccac
ttcaagaactctgtagcaccgcctacatacctcgctctgctaatcctgttaccagtggctgc
tgccagtggcgataagtcgtgtcttaccgggttggactcaagacgatagttaccggataagg
cgcagcggtcgggctgaacggggggttcgtgcacacagcccagcttggagcgaacgacctac
accgaactgagatacctacagcgtgagctatgagaaagcgccacgcttcccgaagggagaaa
ggcggacaggtatccggtaagcggcagggtcggaacaggagagcgcacgagggagcttccag
ggggaaacgcctggtatctttatagtcctgtcgggtttcgccacctctgacttgagcgtcga
tttttgtgatgctcgtcaggggggcggagcctatggaaaaacgccagcaacgcggccttttt
acggttcctggccttttgctggccttttgctcattaggcaccccaggctttacccgaacgac
cgagcgcagcgagtcagtgagcgaggaagcggagagcgcccaatacgcaaggaaacagctat
gaccatgttaatgcagctggcacgacaggtttcccgactggaaagcgggcagtgaaaggaag
gcccatgaggccagctaattaacgatcgagtactaaatgccagtaaagcgctggctgctgaa
cccccagccggaactgaccccacaaggccctagcgtttgcaatgcaccaggtcatcattgac
ccaggcgtgttccaccaggccgctgcctcgcaactcttcgcaggcttcgccgacctgctcgc
gccacttcttcacgcgggtggaatccgatccgcacatgaggcggaaggtttccagcttgagc
gggtacggctcccggtgcgagctgaaatagtcgaacatccgtcgggccgtcggcgacagctt
gcggtacttctcccatatgaatttcgtgtagtggtcgccagcaaacagcacgacgatttcct
cgtcgatcaggacctggcaacgggacgttttcttgccacggtccaggacgcggaagcggtgc
agcagcgacaccgattccaggtgcccaacgcggtcggacgtgaagcccatcgccgtcgcctg
taggcgcgacaggcattcctcggccttcgtgtaataccggccattgatcgaccagcccaggt
cctggcaaagctcgtagaacgtgaaggtgatcggctcgccgataggggtgcgcttcgcgtac
tccaacacctgctgccacaccagttcgtcatcgtcggcccgcagctcgacgccggtgtaggt
gatcttcacgtccttgttgacgtggaaaatgaccttgttttgcagcgcctcgcgcgggattt
tcttgttgcgcgtggtgaacagggcagagcgggccgtgtcgtttggcatcgctcgcatcgtg
tccggccacggcgcaatatcgaacaaggaaagctgcatttccttgatctgctgcttcgtgtg
tttcagcaacgcggcctgcttggcctcgctgacctgttttgccaggtcctcgccggcggttt
ttcgcttcttggtcgtcatagttcctcgcgtgtcgatggtcatcgacttcgccaaacctgcc
gcctcctgttcgagacgacgcgaacgctccacggcggccgatggcgcgggcagggcaggggg
agccagttgcacgctgtcgcgctcgatcttggccgtagcttgctggaccatcgagccgacgg
actggaaggtttcgcggggcgcacgcatgacggtgcggcttgcgatggtttcggcatcctcg
gcggaaaaccccgcgtcgatcagttcttgcctgtatgccttccggtcaaacgtccgattcat
tcaccctccttgcgggattgccccgactcacgccggggcaatgtgcccttattcctgatttg
acccgcctggtgccttggtgtccagataatccaccttatcggcaatgaagtcggtcccgtag
accgtctggccgtccttctcgtacttggtattccgaatcttgccctgcacgaataccagcga
ccccttgcccaaatacttgccgtgggcctcggcctgagagccaaaacacttgatgcggaaga
agtcggtgcgctcctgcttgtcgccggcatcgttgcgccacatatcgattatgatagaattt
acaagctataaggttattgtcctgggtttcaagcattagtccatgcaagtttttatgctttg
cccattctatagatatattgataagcgcgctgcctatgccttgccccctgaaatccttacat
acggcgatatcttctatataaaagatatattatcttatcagtattgtcaatatattcaaggc
aatctgcctcctcatcctcttcatcctcttcgtcttggtagctttttaaatatggcgcttca
tagagtaattctgtaaaggtccaattctcgttttcatacctcggtataatcttacctatcac
ctcaaatggttcgctgggtttatcgcccgggagggttcgagaagggggggcaccccccttcg
gcgtgcgcggtcacgcgcacagggcgcagccctggttaaaaacaaggtttataaatattggt
ttaaaagcaggttaaaagacaggttagcggtggccgaaaaacgggcggaaacccttgcaaat
gctggattttctgcctgtggacagcccctcaaatgtcaataggtgcgcccctcatctgtcag
cactctgcccctcaagtgtcaaggatcgcgcccctcatctgtcagtagtcgcgcccctcaag
tgtcaataccgcagggcacttatccccaggcttgtccacatcatctgtgggaaactcgcgta
aaatcaggcgttttcgccgatttgcgaggctggccagctccacgtcgccggccgaaatcgag
cctgcccctcatctgtcaacgccgcgccgggtgagtcggcccctcaagtgtcaacgtccgcc
cctcatctgtcagtgagggccaagttttccgcgaggtatccacaacgccggcggccgcggtg
tctcgcacacggcttcgacggcgtttctggcgcgtttgcagggccatagacggccgccagcc
cagcggcgagggcaaccagcccggtgagcgtcgcaaaggcgctcggtcttggcgcgccaacc
ctgtggttggcatgcacatacaaatggacgaacggataaaccttttcacgcccttttaaata
tccgattattctaataaacgctcttttctcttaggtttacccgccaatatatcctgtcaaac
actgatagtttaaactgaaggcgggaaacgacaatctgcctgcaggaattgaatt
SEQ ID NO: 2: PQ24F forward primer for nptii
5′-AGCTGTGCTCGACGTTGTCA-3′
SEQ ID NO: 3: PQ24R reverse primer for nptii
5′-CCCCGGCACTTCGCCCAATA-3′
SEQ ID NO: 4: PQ24 Taqman probe for nptii
5′-TGAAGCGGGAAGGGACTGGC-3′
SEQ ID NO: 5: PQ17F forward primer for nitrate reductase gene
5′-GGAAAGAACAGAACATGGTTAAACAA-3′
SEQ ID NO: 6: PQ17R reverse primer for nitrate reductase gene
5′-ACACCGTACCGTTTTAACAAAGC-3′
SEQ ID NO: 7: Taqman probe PQ17 for nitrate reductase gene
5′-TGCCGCTGCCGTTTCAACAACTG-3′
SEQ ID NO: 8: PC201F forward primer
5′-AGAAGGCCTTCCGGGACGGCGTCAG-3′
SEQ ID NO: 9: PC202R reverse primer
5′-ATGGCGCGCCCCCCTCGGGATCA-3′
SEQ ID NO: 10: nucleotide sequence of primer 1
5′-GAGCTGTTGGCTGGCTGG-3′
SEQ ID NO: 11: nucleotide sequence of primer 2
5′-GGCAGGATATATTGTGGTGTAAAC-3′
SEQ ID NO: 12: nucleotide sequence of primer 3
5′-GACCCCCGCCGATGAC-3′
SEQ ID NO: 13: nucleotide sequence of primer 4
5′-CGCAATAATGGTTTCTGACGTA-3′
SEQ ID NO: 14: nucleotide sequence of primer 5
5′-GTGATATTGCTGAAGAGCTTGG-3′
SEQ ID NO: 15: nucleotide sequence of primer 6
5′-TTGCGCGCTATATTTTGTTTTC-3′
SEQ ID NO: 16: nucleotide sequence of primer 7
5′-TAAACGCTCTTTTCTCTTAGGTTTAC-3′
SEQ ID NO: 17: nucleotide sequence of primer 8
5′-AGGCGCTCGGTCTTGG-3′
SEQ ID NO: 18: nucleotide sequence of primer 9
5′-GCGTTGGCTACCCGTGATAT-3′
SEQ ID NO: 19: nucleotide sequence of primer 10
5′-ACATGCTTAACGTAATTCAACAG-3′
SEQ ID NO: 20: minimal 35S-CaMV promoter
gaaacctcctcggattccattgcccagctatctgtcactttattgagaagatagtggaaaag
gaaggtggctcctacaaatgccatcattgcgataaaggaaaggccatcgttgaagatgcctc
tgccgacagtggtcccaaagatggacccccacccacgaggagcatcgtggaaaaagaagacg
ttccaaccacgtcttcaaagcaagtggattgatgtgatatctccactgacgtaagggatgac
gcacaatcccactatccttcgcaagacccttcctctatataaggaagttcatttcatttgga
gagg
SEQ ID NO: 21: 5′UTR HT-CPMV
tattaaaatcttaataggttttgataaaagcgaacgtggggaaacccgaaccaaaccttctt
ctaaactctctctcatctctcttaaagcaaacttctctcttgtctttcttgcgtgagcgatc
ttcaacgttgtcagatcgtgcttcggcaccagtacaacgttttctttcactgaagcgaaatc
aaagatctctttgtggacacgtagtgcggcgccattaaataacgtgtacttgtcctattctt
gtcggtgtggtcttgggaaaagaaagcttgctggaggctgctgttcagccccatacattact
tgttacgattctgctgactttcggcgggtgcaatatctctacttctgcttgacgaggtattg
ttgcctgtacttctttcttcttcttcttgctgattggttctataagaaatctagtattttct
ttgaaacagagttttcccgtggttttcgaacttggagaaagattgttaagcttctgtatatt
ctgcccaaatttgtcgggccc
SEQ ID NO: 22: 3′UTR HT-CPMV
attttctttagtttgaatttactgttattcggtgtgcatttctatgtttggtgagcggtttt
ctgtgctcagagtgtgtttattttatgtaatttaatttctttgtgagctcctgtttagcagg
tcgtcccttcagcaaggacacaaaaagattttaattttattaaaaaaaaaaaaaaaagaccg
gg
SEQ ID NO: 23: P1-HcPro-P3
atggcactcatctttggcacagtcaacgctaacatcctgaaggaagtgttcggtggagctcg
tatggcttgcgttaccagcgcacatatggctggagcgaatggaagcattttgaagaaggcag
aagagacctctcgtgcaatcatgcacaaaccagtgatcttcggagaagactacattaccgag
gcagacttgccttacacaccactccatttagaggtcgatgctgaaatggagcggatgtatta
tcttggtcgtcgcgcgctcacccatggcaagagacgcaaagtttctgtgaataacaagagga
acaggagaaggaaagtggccaaaacgtacgtggggcgtgattccattgttgagaagattgta
gtgccccacaccgagagaaaggttgataccacagcagcagtggaagacatttgcaatgaagc
taccactcaacttgtgcataatagtatgccaaagcgtaagaagcagaaaaacttcttgcccg
ccacttcactaagtaacgtgtatgcccaaacttggagcatagtgcgcaaacgccatatgcag
gtggagatcattagcaagaagagcgtccgagcgagggtcaagagatttgagggctcggtgca
attgttcgcaagtgtgcgtcacatgtatggcgagaggaaaagggtggacttacgtattgaca
actggcagcaagagacacttctagaccttgctaaaagatttaagaatgagagagtggatcaa
tcgaagctcacttttggttcaagtggcctagttttgaggcaaggctcgtacggacctgcgca
ttggtatcgacatggtatgttcattgtacgcggtcggtcggatgggatgttggtggatgctc
gtgcgaaggtaacgttcgctgtttgtcactcaatgacacattatagcgaccatcaccatcac
catcacgcgtccgacaaatcaatctctgaggcattcttcataccatactctaagaaattctt
ggagttgagaccagatggaatctcccatgagtgtacaagaggagtatcagttgagcggtgcg
gtgaggtggctgcaatcctgacacaagcactttcaccgtgtggtaagatcacatgcaaacgt
tgcatggttgaaacacctgacattgttgagggtgagtcgggaggaagtgtcaccaaccaagg
taagctcctagcaatgctgaaagaacagtatccagatttcccaatggccgagaaactactca
caaggtttttgcaacagaaatcactagtaaatacaaatttgacagcctgcgtgagcgtcaaa
caactcattggtgaccgcaaacaagctccattcacacacgtactggctgtcagcgaaattct
gtttaaaggcaataaactaacaggggccgatctcgaagaggcaagcacacatatgcttgaaa
tagcaaggttcttgaacaatcgcactgaaaatatgcgcattggccaccttggttctttcaga
aataaaatctcatcgaaggcccatgtgaataacgcactcatgtgtgataatcaacttgatca
gaatgggaattttatttggggactaaggggtgcacacgcaaagaggtttcttaaaggatttt
tcactgagattgacccaaatgaaggatacgataagtatgttatcaggaaacatatcaggggt
agcagaaagctagcaattggcaatttgataatgtcaactgacttccagacgctcaggcaaca
aattcaaggcgaaactattgagcgtaaagaaattgggaatcactgcatttcaatgcggaatg
gtaattacgtgtacccatgttgttgtgttactcttgaagatggtaaggctcaatattcggat
ctaaagcatccaacgaagagacatctggtcattggcaactctggcgattcaaagtacctaga
ccttccagttctcaatgaagagaaaatgtatatagctaatgaaggttattgctacatgaaca
ttttctttgctctactagtgaatgtcaaggaagaggatgcaaaggacttcaccaagtttata
agggacacaattgttccaaagattggagcgtggccaacaatgcaagatgttgcaactgcatg
ctacttactttccattctttacccagatgtcctgagtgctgaattacccagaattttggttg
atcatgacaacaaaacaatgcatgttttggattcgtatgggtctagaacgacaggataccac
atgttgaaaatgaacacaacatcccagctaattgaattcgttcattcaggtttggaatccga
aatgaaaacttacaatgttggagggatgaaccgagatatggtcacacaaggtgcaattgaga
tgttgatcaagtccatatacaaaccacatctcatgaagcagttacttgaggaggagccatac
ataattgtcctggcaatagtctccccttcaattttaattgccatgtacaactctggaacttt
tgagcaggcgttacaaatgtggttgccaaatacaatgaggttagctaacctcgctgccatct
tgtcagccttggcgcaaaagttaactttggcagacttgttcgtccagcagcgtaatttgatt
aatgagtatgcgcaggtaattttggacaatctgattgacggtgtcagggttaaccattcgct
atccctagcaatggaaattgttactattaagctggccacccaagagatggacatggcgttga
gggaaggtggctatgctgtgacctctgcagatcgttcaaacatttggcaataa
SEQ ID NO: 24: Influenza haemagglutinin 5 (H5)
atggagaaaatagtgcttcttcttgcaatagtcagtcttgttaaaagtgatcagatttgcat
tggttaccatgcaaacaattcaacagagcaggttgacacaatcatggaaaagaacgttactg
ttacacatgcccaagacatactggaaaagacacacaacgggaagctctgcgatctagatgga
gtgaagcctctaattttaagagattgtagtgtagctggatggctcctcgggaacccaatgtg
tgacgaattcatcaatgtaccggaatggtcttacatagtggagaaggccaatccaaccaatg
acctctgttacccagggagtttcaacgactatgaagaactgaaacacctattgagcagaata
aaccattttgagaaaattcaaatcatccccaaaagttcttggtccgatcatgaagcctcatc
aggagttagctcagcatgtccatacctgggaagtccctccttttttagaaatgtggtatggc
ttatcaaaaagaacagtacatacccaacaataaagaaaagctacaataataccaaccaagag
gatcttttggtactgtggggaattcaccatcctaatgatgcggcagagcagacaaggctata
tcaaaacccaaccacctatatttccattgggacatcaacactaaaccagagattggtaccaa
aaatagctactagatccaaagtaaacgggcaaagtggaaggatggagttcttctggacaatt
ttaaaacctaatgatgcaatcaacttcgagagtaatggaaatttcattgctccagaatatgc
atacaaaattgtcaagaaaggggactcagcaattatgaaaagtgaattggaatatggtaact
gcaacaccaagtgtcaaactccaatgggggcgataaactctagtatgccattccacaacata
caccctctcaccatcggggaatgccccaaatatgtgaaatcaaacagattagtccttgcaac
agggctcagaaatagccctcaaagagagagcagaagaaaaaagagaggactatttggagcta
tagcaggttttatagagggaggatggcagggaatggtagatggttggtatgggtaccaccat
agcaatgagcaggggagtgggtacgctgcagacaaagaatccactcaaaaggcaatagatgg
agtcaccaataaggtcaactcaatcattgacaaaatgaacactcagtttgaggccgttggaa
gggaatttaataacttagaaaggagaatagagaatttaaacaagaagatggaagacgggttt
ctagatgtctggacttataatgccgaacttctggttctcatggaaaatgagagaactctaga
ctttcatgactcaaatgttaagaacctctacgacaaggtccgactacagcttagggataatg
caaaggagctgggtaacggttgtttcgagttctatcacaaatgtgataatgaatgtatggaa
agtataagaaacggaacgtacaactatccgcagtattcagaagaagcaagattaaaaagaga
ggaaataagtggggtaaaattggaatcaataggaacttaccaaatactgtcaatttattcaa
cagtggcgagttccctagcactggcaatcatgatggctggtctatctttatggatgtgctcc
aatggatcgttacaatgcagaatttgcatttaa
SEQ ID NO: 25: pMMV single enhanced between EcoR1 and
Hindi sites [symbol]
gtcaacttcgtccacagacatcaacatcttatcgtcctttgaagataagataataa
tgttgaagataagagtgggagccaccactaaaacattgctttgtcaaaagctaaaaaagatg
atgcccgacagccacttgtgtgaagcatgagaagccggtccctccactaagaaaattagtga
agcatcttccagtggtccctccactcacagctcaatcagtgagcaacaggacgaaggaaatg
acgtaagccatgacgtctaatcccacaagaatttccttatataaggaacacaaatcagaagg
aagagatcaatcgaaatcaaaatcggaatcgaaatcaaaatcggaatcgaaatctctcatct

[symbol]

SEQ ID NO: 26: pMMV double enhanced between EcoR1 and
Hind3 sites [symbol]
gtcaacttcgtccacagacatcaacatcttatcgtcctttgaagataagataataa
tgttgaagataagagtgggagcccccactaaaacattgctttgtcaaaagctaaaaaagatg
atgcccgacagccacttgtgtgaagcatgagaagccggtccctccactaagaaaattagtga
agcatcttccagtggtccctccactcacagctcaatcagtgagcaacaggacgaaggaaatg
acgtaagccatgacgtctaatcccaacttcgtccacagacatcaacatcttatcgtcctttg
aagataagataataatgttgaagataagagtgggagccaccactaaaacattgctttgtcaa
aagctaaaaaagatgatgcccgacagccacttgtgtgaagcatgagaagccggtccctccac
taagaaaattagtgaagcatcttccagtggtccctccactcacagctcaatcagtgagcaac
aggacgaaggaaatgacgtaagccatgacgtctaatcccacaagaatttccttatataagga
acacaaatcagaaggaagagatcaatcgaaatcaaaatcggaatcgaaatcaaaatcggaat
cgaaatctctcatct [symbol]
SEQ ID NO: 27: pFMV single enhanced between EcoR1 and
Hind3 sites [symbol]
gtcaacatcgagcagctggcttgtggggaccagacaaaaaaggaatggtgcagaat
tgttaggcgcacctaccaaaagcatctttgcctttattgcaaagataaagcagattcctcta
gtacaagtggggaacaaaataacgtggaaaagagctgtcctgacagcccactcactaatgcg
tatgacgaacgcagtgacgaccacaaaagattgcccgggtaatccctctatataagaaggca
ttcattcccatttgaaggatcatcagatactcaaccaatatttctcactctaagaaattaag
agctttgtattcttcaatgagggctaagaccc [symbol]
SEQ ID NO: 28: pFMV double enhanced between EcoR1 and
Hind3 sites [symbol]
gtcaacatcgagcagctggcttgtggggaccagacaaaaaaggaatggtgcagaat
tgttaggcgcacctaccaaaagcatctttgcctttattgcaaagataaagcagattcctcta
gtacaagtggggaacaaaataacgtggaaaagagctgtcctgacagcccactcactaatgcg
tatgacgaacgcagtgacgaccacaaaagattgcccaacatcgagcagctggcttgtgggga
ccagacaaaaaaggaatggtgcagaattgttaggcgcacctaccaaaagcatctttgccttt
attgcaaagataaagcagattcctctagtacaagtggggaacaaaataacgtggaaaagagc
tgtcctgacagcccactcactaatgcgtatgacgaacgcagtgacgaccacaaaagattgcc
cgggtaatccctctatataagaaggcattcattcccatttgaaggatcatcagatactcaac
caatatttctcactctaagaaattaagagctttgtattcttcaatgagaggctaagaccc

[symbol]

[symbol]

SEQ ID NO: 29: pPCISV single enhanced between EcoR1 and
Hind3 sites [symbol]
aattcgtcaacgagatcttgagccaatcaaagaggagtgatgttgacctaaagcaa
taatggagccatgacgtaagggcttacgcccatacgaaataattaaaggctgatgtgacctg
tcggtctctcagaacctttactttttatatttggcgtgtatttttaaatttccacggcaatg
acgatgtgacctgtgcatccgctttgcctataaataagttttagtttgtattgatcgacacg
atcgagaagacacggccata [symbol]
SEQ ID NO: 30: pPCISV double enhanced between EcoR1 and
Hind3 sites [symbol]
gtcaacgagatcttgagccaatcaaagaggagtgatgtagacctaaagcaataatg
gagccatgacgtaagggcttacgcccatacgaaataattaaaggctgatgtgacctgtcggt
ctctcagaacctttactttttatgtttggcgtgtatttttaaatttccacggcaatgacgat
gtgacccaacgagatcttgagccaatcaaagaggagtgatgtagacctaaagcaataatgga
gccatgacgtaagggcttacgcccatacgaaataattaaaggctgatgtgacctgtcggtct
ctcagaacctttactttttatatttggcgtgtatttttaaatttccacggcaatgacgatgt
gacctgtgcatccgctttgcctataaataagttttagtttgtattgatcgacacggtcgaga
agacacggccat [symbol]
SEQ 1D NO: 31: patatin signal peptide
MATTKSFLILFFMILATTSSTCA
SEQ ID NO: 32: rituximab mature heavy chain
(tobacco optimized) sequence
caagttcaacttcaacaaccaggtgctgaacttgttaagcctggtgcttctgttaagatgtc
ttgcaaggcttctggatacactttcacatcctacaacatgcattgggttaagcaaactccag
gacgtggacttgaatggattggagctatctaccctggaaacggtgatacttcctacaaccag
aagttcaagggaaaggctactattactgctgataagtcctcttccactgcttacatgcaact
ttcttcactcacttccgaggattctgctgtttattactgcgctaggtccacttattatggtg
gagattggtacttcaatgtttggggagctggaactactgttactgtgtctgctgcttctact
aagggaccatctgtttttccacttgctccatcttctaagtctacttccggtggaactgctgc
tcttggatgccttgtgaaggattatttcccagagccagtgactgtttcttggaactctggtg
ctcttacttctggtgttcacactttcccagatgttcttcagtcatctggactttactccctt
tcttatgttgttactgtgccatcttcttcacttggaactcagacttacatctgcaacgttaa
ccacaagccatctaacacaaaagtggataagaaggcagagccaaagtcttgtgataagactc
atacttgtccaccatgtccagctccagaacttcttggtggtccatctgttttcttgttccca
ccaaagccaaaggatactctcatgatctctaggactccagaagttacttgcgttgttgtgga
tgtttctcatgaggacccagaggttaagttcaactggtacgtggatggtgttgaagttcaca
acgctaagactaagccaagataggaacagtacaactctacttaccgtgttgtgtctgtgctt
actgttcttcaccaggattggcttaacggaaaagagtacaaatgcaaggtttccaataaggc
tttaccagctccaattgaaaagactatctccaaggcaaaaggacagcctagagagccacagg
tttacactcttccaccatctagagatgagcttactaagaaccaggtttcccttacttgtctt
gtgaagggattctacccatctgatattgctgttgagtgggagtcaaacggacagcctgagaa
caactacaagactactccaccagtgcttgattctgatggttccttcttcctctactccaaac
tcactgtggataagtctagatggcagcagggaaatgttttctcttgctccgttatgcatgag
gctctccataatcactacactcagaagtccctttctttgtctcctggaaagtga
SEQ ID NO: 33: rituximab mature heavy chain amino
acid sequence
QVQLQQPGAELVKPGASVKMSCKASGYTFTSYNMHWVKQTPGRGLEWIGAIYPGNGDTSYNQ
KFKGKATLTADKSSSTAYMQLSSLTSEDSAVYYCARSTYYGGDWYFNVWGAGTTVTVSAAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKAEPKSCDKTHTCPPCPAPELLGGPSVFLFP
PKPKDTLMISRTPEVTCVVVDVSHEDPEVKFNWYVDGVEVHNAKTKPR*EQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK*
SEQ ID NO: 34: patatin tobacco non optimized sequence
(slightly modified) as in C148 (in front of heavy chain)
atggccactactaaatcttttttaattttattttttatgatattagcaactactagttcaac
atgtgct
SEQ ID NO: 35: rituximab mature light chain
(tobacco optimized) sequence
cagattgtgctttctcagtctccagctattctttctgcttccccaggtgaaaaggttacaat
gacttgccgtgcttcttcttctgtgtcctacattcattggttccaacagaagccaggatctt
ctccaaagccatggatctacgctacttctaaccttgcttctggtgttccagttaggttttct
ggatctggatctggtacttcttactcccttactatttctagagtggaggctgaagatgctgc
tacttactactgccaacagtggacttctaatccaccaactttcggaggtggaactaagcttg
agatcaagaggactgttgctgctccatctgtgtttattttcccaccatctgatgagcaactt
aagtctggaactgcttctgttgtgtgccttctcaacaatttctacccaagggaagctaaggt
tcagtggaaagtggataatgctctccagtctggaaattctcaagagtctgtgactgagcagg
attctaaggattccacttactccctttcttctactcttactctctccaaggctgattatgag
aagcacaaggtttacgattgcgaagttactcatcagggactttcttcaccagtgacaaagtc
cttcaaccgtggagagtgttga
SEQ ID NO: 36: rituximab mature light chain amino
aicd sequence
QIVLSQSPAILSASPGEKVTMTCRASSSVSYIHWFQQKPGSSPKPWIYATSNLASGVPVRFS
GSGSGTSYSLTISRVEAEDAATYYCQQWTSNPPTFGGGTKLEIKRTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC*
SEQ ID NO: 37: patatin tobacco optimized sequence
as in C148 (in front of light chain)
atggccactactaagtccttccttatcctcttcttcatgatccttgctactacttcttctac
atgtgct
SEQ ID NO: 38: mature GBA (tobacco optimized) sequence
gctagaccatgcattcctaagtctttcggttactcttctgttgtgtgcgtgtgcaatgctac
ttactgcgattctttcgatcctcctacttttcctgctcttggtactttttctaggtacgagt
ctaccaggtctggtagaagaatggaactttctatgggtcctatccaggctaatcatactggt
actggtctgcttcttactcttcaacctgagcagaagttccaaaaggttaagggttttggtgg
tgctatgactgatgctgctgctcttaatattctggctctttctcctcctgctcaaaacttgc
tgctgaagtcttacttcagcgaagagggtatcggttacaacattattagggtgccaatggct
tcctgcgatttctctattaggacttatacctacgctgatacccctgatgatttccagcttca
caactttagcctgcctgaagaggataccaagctgaagattcctcttattcatagggctctgc
agcttgctcaaagacctgtttctcttttggcttctccttggacttctcctacttggcttaag
actaatggtgctgtgaacggtaagggttctcttaagggtcaacctggtgatatctaccatca
aacttgggctagatacttcgtgaagttccttgatgcttacgctgagcataagttgcagtttt
gggctgttactgctgagaatgagccttctgctggtcttttgtctggttatccttttcagtgc
cttggtttcactcctgaacatcagagggatttcattgctagagatttgggtcctacccttgc
taattctactcatcataacgtgaggctgctgatgcttgatgatcagagacttcttttgcctc
actgggctaaggttgtgcttactgatcctgaagctgctaagtacgttcacggtattgctgtt
cactggtacttggattttctggctcctgctaaggctactcttggtgaaactcataggctttt
ccctaacaccatgctttttgcttcagaggcttgcgttggttctaagttttgggaacagtctg
tgagacttggatcttgggatagaggtatgcagtacagccactctattattaccaacctgctg
taccatgtggtgggttggactgattggaatcttgctcttaatcctgagggtggtcctaattg
ggttaggaatttcgtggatagccctatcatcgtggatattaccaaggataccttctacaagc
agcctatgttctaccatcttggtcacttcagcaagttcattccagaaggttctcagagggtt
ggacttgttgcttctcaaaagaacgatcttgatgctgtggctcttatgcaccctgatggttc
tgctgttgttgttgtgcttaacaggtctagcaaggatgtgcctctgactatcaaagatcctg
ctgttggtttcttagagaccatttctcctggttactctattcacacctacctttggcgtcga
caa
SEQ ID NO: 39: mature GBA amino acid sequence
ARPCIPKSFGYSSVVCVCNATYCDSFDPPTFPALGTFSRYESTRSGRRMELSMGPIQANHTG
TGLLLTLQPEQKFQKVKGFGGAMTDAAALNILALSPPAQNLLLKSYFSEEGIGYNIIRVPMA
SCDFSIRTYTYADTPDDFQLHNFSLPEEDTKLKIPLIHRALQLAQRPVSLLASPWTSPTWLK
TNGAVNGKGSLKGQPGDIYHQTWARYFVKFLDAYAEHKLQFWAVTAENEPSAGLLSGYPFQC
LGFTPEHQRDFIARDLGPTLANSTHHNVRLLMLDDQRLLLPHWAKVVLTDPEAAKYVHGIAV
HWYIDFLAPAKATLGETHRLFPNTMLFASEACVGSKFWEQSVRLGSWDRGMQYSHSIITNLL
YHVVGWTDWNLALNPEGGPNWVRNFVDSPIIVDITKDTFYKQPMFYHLGHFSKFIPEGSQRV
GLVASQKNDLDAVALMHPDGSAVVVVLNRSSKDVPLTIKDPAVGFLETISPGYSIHTYLWRR
Q
SEQ ID NO: 40: patatin tobacco optimized sequence
in front of GBA
atggctactactaagtctttcctgatcctgttcttcatgattcttgctactacctcgagcac
gtgtgct

### REFERENCES

Alberts et al. (2002). Molecular Biology of the Cell, 4^{th} edition. Garland Science, New York. ISBN: 0-8153-3218-1
Bevan (1984) Binary Agrobacterium vectors for plant transformation. Nucl. Acids. Res. 12: 8711-8721.
De Buck et al. (2000) T-DNA vector backbone sequences are frequently integrated into the genome of transgenic plants obtained by Agrobacterium-mediated transformation. Molecular Breeding 6: 459-468.
Fraley et al. (1983) Expression of bacterial genes in plant cells. Proc. Natl. Acad. Sci. USA 80: 4803-4807.
Hajdukiewicz et al. (1994) The small, versatile pPZP family of Agrobacterium binary vectors for plant transformation. Plant. Mol. Biol. 25: 989-994.
Ingham et al. (2001) Quantitative real-time PCR assay for determining transgene copy number in transformed plants. Biotechniques 31: 132-140.
Kosonov et al. (1997) Integration of T-DNA binary vector “backbone” sequences into the tobacco genome: eividence for multiple complex patterns of integration. Plant J. 11: 945-957.
Lee and Gelvin (2008) T-DNA binary vectors and systems. Plant Physiology 146: 325-332.
Liu et al. (1999) Complementation of plant mutants with large genomic DNA fragments by a transformation-competent artificial chromosome vector accelerates positional cloning. Proc. Natl. Acad. Sci. USA 96: 6535-6540.
Ramanathan and Veluthambi, 1995. Plant Mol. Biol. 28: 1149-1154)
Schmidhauser and Helinski (1985) Regions of broad-host-range plasmid RK2 involved in replication and stable maintenance in nine species of gram-negative bacteria. J. Bacterial. 164: 446-455.
Wenck et al. (1997) Frequent colinear long transfer of DNA inclusive of the whole binary vector during Agrobacterium-mediated transformation. Plant Mol. Biol. 34: 913-922.
Zambryski et al. (1983) Ti plasmid vector for the introduction of DNA into plant cells without alteration of their normal regeneration capacity. EMBO J. 2: 2143-2150.

1. A vector molecule comprising the following nucleic acid elements:
a) a first nucleic acid element comprising a nucleotide sequence encoding a selectable marker which is functional in Escherichia coli and Agrobacterium species;
b) a second nucleic acid element comprising a nucleotide sequence of a first origin of replication which is functional in Escherichia coli;
c) a third nucleic acid element comprising a nucleotide sequence encoding a replication initiator protein;
d) a fourth nucleic acid element comprising a nucleotide sequence of a second origin of replication, which is different from the first origin of replication and which is functional in Agrobacterium; and
e) a fifth nucleic acid element comprising a nucleotide sequence of a T-DNA region comprising a T-DNA right border sequence and a T-DNA left border sequence of a tumour-inducing Agrobacterium tumefaciens plasmid or a root-inducing plasmid of Agrobacterium rhizogenes;
wherein the above nucleic acid elements are provided on a circular polynucleotide molecule and are separated by gap nucleotide sequences which have no function in replication, maintenance or nucleic acid transfer, and wherein said gap nucleotide sequences account for less than 30% of the total vector size.

2. The vector molecule of claim 1, which has a total size of less than 5500 bp.

3. The vector molecule of claim 1, wherein the nucleic acid elements (a) to (e) are arranged on the vector molecule in the order set out in claim 1.

4. The vector molecule of claim 1, wherein
a) the T-DNA left border sequence and the nucleotide sequence encoding a selectable marker (a) is separated by a first gap nucleotide sequence of not more than 300 bp;
b) the nucleotide sequence encoding a selectable marker (a) and the nucleotide sequence of a first origin of replication (b) is separated by a second gap nucleotide sequence of not more than 200 bp;
c) the nucleotide sequence of a first origin of replication (b) and the nucleotide sequence encoding a replication initiator protein (c) is separated by a third gap nucleotide sequence of not more than 200 bp;
d) the nucleotide sequence encoding a replication initiator protein (c) and the nucleotide sequence of a second origin of replication (d) is separated by a fourth gap nucleotide sequence of not more than 500 bp; and
e) the nucleotide sequence of a second origin of replication (d) and the T-DNA right border sequence is separated by a fifth gap nucleotide sequence of not more than 150 bp.

5. The vector molecule of claim 1, wherein the first nucleic acid element (a) comprises a nucleotide sequence encoding for an antibiotic resistance, wherein said antibiotic is selected from the group consisting of ampicillin, chloramphenicol, kanamycin, tetracycline, gentamycin, spectinomycin, bleomycin, phleomycin, rifampicin, streptomycin and blasticidin S.

6. The vector molecule of claim 1, wherein the second nucleic acid element (b) comprises a nucleotide sequence of a first origin of replication selected from the group consisting of a ColE1 origin of replication or an origin of replication belonging to any of incompatibility group FI, FII, FIII, FIV, I J, N, O, P, Q, T, or W.

7. The vector molecule of claim 1, wherein the fourth nucleic acid element (d) comprises a nucleotide sequence of a second origin of replication which is a minimal oriV origin of replication.

8. The vector molecule of claim 1, wherein the fifth nucleic acid element (e) comprises at least one unique restriction endonuclease cleavage site.

9. The vector molecule according to claim 1, wherein the fifth nucleic acid element further comprises, between the T-DNA right and T-DNA left border sequences, a regulatory element which is functional in a plant cell.

10. The vector molecule according to claim 1 having a polynucleotide sequence being at least 80% identical to the polynucleotide sequence as depicted in SEQ ID NO: 1 and wherein the nucleic acid elements (a) to (e) exhibit the same functionality as the counterpart elements provided in SEQ ID NO:1

11. The vector molecule according to claim 1, wherein the fifth nucleic acid element further comprises, between the T-DNA right and T-DNA left border sequences, a nucleotide sequence encoding a protein of interest which is operably linked to a regulatory element which is functional in a plant cell.

12. The vector molecule according to claim 11, wherein the nucleotide sequence encoding the protein of interest is selected from the group consisting of a growth factor, a receptor, a ligand, a signaling molecule; a kinase, an enzyme, a hormone, a tumor suppressor, a blood clotting protein, a cell cycle protein, a metabolic protein, a neuronal protein, a cardiac protein, a protein deficient in specific disease states, an antibody or a fragment thereof, an antigen, a protein that provides resistance to an infectious disease, an antimicrobial protein, an interferon, and a cytokine.

13. The vector molecule according to claim 11, wherein the nucleotide sequence encoding the protein of interest is a suppressor of gene silencing.

14. The vector molecule according to claim 11, wherein the nucleotide sequence encoding the protein of interest is influenza haemagglutinin 5 (H5) as shown in SEQ ID NO: 24.

15. The vector molecule according to claim 11, wherein the nucleotide sequence encoding the protein of interest is a nucleotide sequence encoding a light chain of an antibody, a heavy chain of an antibody, or both a light chain and a heavy chain of an antibody, wherein said heavy chain or light chain is that of an antibody that binds human CD20 with the antibody binding site of a rituximab.

16. The vector molecule according to claim 15, wherein said nucleotide sequence encodes the mature heavy chain of an immunoglobulin that binds human CD20, and exhibits at least 90%, 92%, 94%, 96%, 98%, 99% or 99.5% sequence identity to SEQ ID NO: 32.

17. The vector molecule according to claim 15, wherein said nucleotide sequence encodes the mature light chain of an immunoglobulin that binds human CD20 and exhibits at least 90%, 92%, 94%, 96%, 98%, 99% or 99.5% sequence identity to SEQ ID NO: 35.

18. The vector molecule according to claim 11, wherein the nucleotide sequence encoding the protein of interest comprises a sequence that has been optimized for expression in plant cells.

19. The vector molecule of claim 18, wherein one or more codons in the nucleotide sequence encoding the protein of interest have been replaced with plant preferred codons.

20. The vector molecule according to claim 11, wherein the nucleotide sequence encoding a protein of interest is operably linked to a—enhanced FLt promoter from Mirabilis Mosaic Virus (pMMV 2×).

21. A method for producing a heterologous polypeptide in a plant, particularly a Nicotiana tabacum plant, comprising the steps of:
(i) providing a combination of a selected variety, breeding line, or cultivar and a selected Agrobacterium strain comprising a vector according to claim 11;
(ii) infiltrating a whole plant of the selected variety, breeding line, or cultivar with a bacterial suspension of the selected Agrobacterium strain;
(iii) incubating the infiltrated plant for a period of between 5 days and 10 days under conditions that allow expression of the expressible nucleotide sequence in the infiltrated plant and accumulation of the protein of interest.

22. A method for producing a protein of interest in a plant cell comprising introducing into a plant cell at least one vector of claim 11 and incubating the plant cell to allow production of the protein of interest.

23. A plant cell comprising the fifth nucleic acid element according to claim 11 wherein between the T-DNA right and T-DNA left border sequences is a nucleotide sequence encoding the protein of interest.

24. A plant cell prepared according to claim 23 comprising a nucleotide sequence encoding the protein of interest.


SEQUENCE LISTING (40 sequences)

SEQ ID NO: 1 - DNA, 5139 bp, Artificial sequence
  source: /note=“Description of artificial sequence vector pPMP1”
ctactagtcccctagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcaggccttgacggccttccttcaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaggatggcctgcatgcgggtagcccggcagcgtgggtggaacgtctgtttggctatgattgggcgcagcagaccattggctgctctgatgcggcggtgtttcgtctgagcgcgcagggtcgtccggtgctgtttgtgaaaaccgatctgagcggtgcgctgaacgagctgcaggatgaagcggcgcgtctgagctggctggccaccaccggtgttccgtgtgcggcggtgctggatgtggtgaccgaagcgggccgtgattggctgctgctgggcgaagtgccgggtcaggatctgctgtctagccatctggcgccggcagaaaaagtgagcattatggcggatgccatgcgtcgtctgcataccctggacccggcgacctgtccgtttgatcatcaggcgaaacatcgtattgaacgtgcgcgtacccgtatggaagcgggcctggtggatcaggatgatctggatgaagaacatcagggcctggcaccggcagagctgtttgcgcgtctgaaagcgagcatgccggatggcgaagatctggtggtgacccatggtgatgcgtgcctgccgaacattatggtggaaaatggccgttttagcggctttattgattgcggccgtctgggcgtggcggatcgttatcaggatattgcgctggccacccgtgatattgcggaagaactgggcggcgaatgggcggatcgttttctggtgctgtatggcattgcggcaccggatagccagcgtattgcgttttatcgtctgctggatgaatttttctaataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgaaaggaaggcccatgaggccagttaattaacgatcgagtactaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatatcgattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttggcgcgccaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgcctgcaggaattgaatt

SEQ ID NO: 2 - DNA, 20 bp, Artificial sequence
  source: /note=“Description of artificial sequence forward primer for nptii”
agctgtgctcgacgttgtca

SEQ ID NO: 3 - DNA, 20 bp, Artificial sequence
  source: /note=“Description of artificial sequence reverse primer for nptii”
ccccggcacttcgcccaata

SEQ ID NO: 4 - DNA, 20 bp, Artificial sequence
  source: /note=“Description of artificial sequence Taqman probe for nptii”
tgaagcgggaagggactggc

SEQ ID NO: 5 - DNA, 26 bp, Artificial sequence
  source: /note=“Description of artificial sequence forward primer for nitrate reductase gene”
ggaaagaacagaacatggttaaacaa

SEQ ID NO: 6 - DNA, 23 bp, Artificial sequence
  source: /note=“Description of artificial sequence reverse primer for nitrate reductase gene”
acaccgtaccgttttaacaaagc

SEQ ID NO: 7 - DNA, 23 bp, Artificial sequence
  source: /note=“Description of artificial sequence Taqman probe PQ17 for nitrate reductase gene”
tgccgctgccgtttcaacaactg

SEQ ID NO: 8 - DNA, 25 bp, Artificial sequence
  source: /note=“Description of artificial sequence forward primer”
agaaggccttccgggacggcgtcag

SEQ ID NO: 9 - DNA, 23 bp, Artificial sequence
  source: /note=“Description of artificial sequence reverse primer”
atggcgcgcccccctcgggatca

SEQ ID NO: 10 - DNA, 18 bp, Artificial sequence
  source: /note=“Description of artificial sequence primer 1”
gagctgttggctggctgg

SEQ ID NO: 11 - DNA, 24 bp, Artificial sequence
  source: /note=“Description of artificial sequence primer 2”
ggcaggatatattgtggtgtaaac

SEQ ID NO: 12 - DNA, 16 bp, Artificial sequence
  source: /note=“Description of artificial sequence primer 3”
gacccccgccgatgac

SEQ ID NO: 13 - DNA, 22 bp, Artificial sequence
  source: /note=_“Description of artificial sequence primer 4”
cgcaataatggtttctgacgta

SEQ ID NO: 14 - DNA, 22 bp, Artificial sequence
  source: /note=“Description of artificial sequence primer 5”
gtgatattgctgaagagcttgg

SEQ ID NO: 15 - DNA, 22 bp, Artificial sequence
  source: /note=“Description of artificial sequence primer 6”
ttgcgcgctatattttgttttc

SEQ ID NO: 16 - DNA, 26 bp, Artificial sequence
  source: /note=“Description of artificial sequence primer 7”
taaacgctcttttctcttaggtttac

SEQ ID NO: 17 - DNA, 16 bp, Artificial sequence
  source: /note=“Description of artificial seuqence primer 8”
aggcgctcggtcttgg

SEQ ID NO: 18 - DNA, 20 bp, Artificial sequence
  source: /note=“Description of artificial sequence primer 9”
gcgttggctacccgtgatat

SEQ ID NO: 19 - DNA, 23 bp, Artificial sequence
  source: /note=“Description of artificial sequence primer 10”
acatgcttaacgtaattcaacag

SEQ ID NO: 20 - DNA, 314 bp, artificial sequence
  source: /note=“Description of artificial sequence minimal 35S-CaMV promoter”
gaaacctcctcggattccattgcccagctatctgtcactttattgagaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagg

SEQ ID NO: 21 - DNA, 517 bp, artificial sequence
  source: /note=“Description of artificial sequence 5 UTR HT-CPMV”
tattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggccc

SEQ ID NO: 22 - DNA, 188 bp, artificial sequence
  source: /note=“Description of artificial sequence 3 UTR HT-CPMV”
attttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaagaccggg

SEQ ID NO: 23 - DNA, 2781 bp, artificial sequence
  source: /note=“Description of artificial sequence P1-HcPro-P3”
atggcactcatctttggcacagtcaacgctaacatcctgaaggaagtgttcggtggagctcgtatggcttgcgttaccagcgcacatatggctggagcgaatggaagcattttgaagaaggcagaagagacctctcgtgcaatcatgcacaaaccagtgatcttcggagaagactacattaccgaggcagacttgccttacacaccactccatttagaggtcgatgctgaaatggagcggatgtattatcttggtcgtcgcgcgctcacccatggcaagagacgcaaagtttctgtgaataacaagaggaacaggagaaggaaagtggccaaaacgtacgtggggcgtgattccattgttgagaagattgtagtgccccacaccgagagaaaggttgataccacagcagcagtggaagacatttgcaatgaagctaccactcaacttgtgcataatagtatgccaaagcgtaagaagcagaaaaacttcttgcccgccacttcactaagtaacgtgtatgcccaaacttggagcatagtgcgcaaacgccatatgcaggtggagatcattagcaagaagagcgtccgagcgagggtcaagagatttgagggctcggtgcaattgttcgcaagtgtgcgtcacatgtatggcgagaggaaaagggtggacttacgtattgacaactggcagcaagagacacttctagaccttgctaaaagatttaagaatgagagagtggatcaatcgaagctcacttttggttcaagtggcctagttttgaggcaaggctcgtacggacctgcgcattggtatcgacatggtatgttcattgtacgcggtcggtcggatgggatgttggtggatgctcgtgcgaaggtaacgttcgctgtttgtcactcaatgacacattatagcgaccatcaccatcaccatcacgcgtccgacaaatcaatctctgaggcattcttcataccatactctaagaaattcttggagttgagaccagatggaatctcccatgagtgtacaagaggagtatcagttgagcggtgcggtgaggtggctgcaatcctgacacaagcactttcaccgtgtggtaagatcacatgcaaacgttgcatggttgaaacacctgacattgttgagggtgagtcgggaggaagtgtcaccaaccaaggtaagctcctagcaatgctgaaagaacagtatccagatttcccaatggccgagaaactactcacaaggtttttgcaacagaaatcactagtaaatacaaatttgacagcctgcgtgagcgtcaaacaactcattggtgaccgcaaacaagctccattcacacacgtactggctgtcagcgaaattctgtttaaaggcaataaactaacaggggccgatctcgaagaggcaagcacacatatgcttgaaatagcaaggttcttgaacaatcgcactgaaaatatgcgcattggccaccttggttctttcagaaataaaatctcatcgaaggcccatgtgaataacgcactcatgtgtgataatcaacttgatcagaatgggaattttatttggggactaaggggtgcacacgcaaagaggtttcttaaaggatttttcactgagattgacccaaatgaaggatacgataagtatgttatcaggaaacatatcaggggtagcagaaagctagcaattggcaatttgataatgtcaactgacttccagacgctcaggcaacaaattcaaggcgaaactattgagcgtaaagaaattgggaatcactgcatttcaatgcggaatggtaattacgtgtacccatgttgttgtgttactcttgaagatggtaaggctcaatattcggatctaaagcatccaacgaagagacatctggtcattggcaactctggcgattcaaagtacctagaccttccagttctcaatgaagagaaaatgtatatagctaatgaaggttattgctacatgaacattttctttgctctactagtgaatgtcaaggaagaggatgcaaaggacttcaccaagtttataagggacacaattgttccaaagcttggagcgtggccaacaatgcaagatgttgcaactgcatgctacttactttccattctttacccagatgtcctgagtgctgaattacccagaattttggttgatcatgacaacaaaacaatgcatgttttggattcgtatgggtctagaacgacaggataccacatgttgaaaatgaacacaacatcccagctaattgaattcgttcattcaggtttggaatccgaaatgaaaacttacaatgttggagggatgaaccgagatatggtcacacaaggtgcaattgagatgttgatcaagtccatatacaaaccacatctcatgaagcagttacttgaggaggagccatacataattgtcctggcaatagtctccccttcaattttaattgccatgtacaactctggaacttttgagcaggcgttacaaatgtggttgccaaatacaatgaggttagctaacctcgctgccatcttgtcagccttggcgcaaaagttaactttggcagacttgttcgtccagcagcgtaatttgattaatgagtatgcgcaggtaattttggacaatctgattgacggtgtcagggttaaccattcgctatccctagcaatggaaattgttactattaagctggccacccaagagatggacatggcgttgagggaaggtggctatgctgtgacctctgcagatcgttcaaacatttggcaataa

SEQ ID NO: 24 - DNA, 1707 bp, artificial sequence
  source: /note=“Description of artificial sequence Mature Optimized HEI (H5)”
atggagaaaatagtgcttcttcttgcaatagtcagtcttgttaaaagtgatcagatttgcattggttaccatgcaaacaattcaacagagcaggttgacacaatcatggaaaagaacgttactgttacacatgcccaagacatactggaaaagacacacaacgggaagctctgcgatctagatggagtgaagcctctaattttaagagattgtagtgtagctggatggctcctcgggaacccaatgtgtgacgaattcatcaatgtaccggaatggtcttacatagtggagaaggccaatccaaccaatgacctctgttacccagggagtttcaacgactatgaagaactgaaacacctattgagcagaataaaccattttgagaaaattcaaatcatccccaaaagttcttggtccgatcatgaagcctcatcaggagttagctcagcatgtccatacctgggaagtccctccttttttagaaatgtggtatggcttatcaaaaagaacagtacatacccaacaataaagaaaagctacaataataccaaccaagaggatcttttggtactgtggggaattcaccatcctaatgatgcggcagagcagacaaggctatatcaaaacccaaccacctatatttccattgggacatcaacactaaaccagagattggtaccaaaaatagctactagatccaaagtaaacgggcaaagtggaaggatggagttcttctggacaattttaaaacctaatgatgcaatcaacttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccctctcaccatcggggaatgccccaaatatgtgaaatcaaacagattagtccttgcaacagggctcagaaatagccctcaaagagagagcagaagaaaaaagagaggactatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcaatcattgacaaaatgaacactcagtttgaggccgttggaagggaatttaataacttagaaaggagaatagagaatttaaacaagaagatggaagacgggtttctagatgtctggacttataatgccgaacttctggttctcatggaaaatgagagaactctagactttcatgactcaaatgttaagaacctctacgacaaggtccgactacagcttagggataatgcaaaggagctgggtaacggttgtttcgagttctatcacaaatgtgataatgaatgtatggaaagtataagaaacggaacgtacaactatccgcagtattcagaagaagcaagattaaaaagagaggaaataagtggggtaaaattggaatcaataggaacttaccaaatactgtcaatttattcaacagtggcgagttccctagcactggcaatcatgatggctggtctatctttatggatgtgctccaatggatcgttacaatgcagaatttgcatttaa

SEQ ID NO: 25 - DNA, 378 bp, artificial sequence
  source: /note=“Description of artificial sequence pMMV single enhanced between EcoR1 and Hind3 sites”
gaattcgtcaacttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgagaagccggtccctccactaagaaaattagtgaagcatcttccagtggtccctccactcacagctcaatcagtgagcaacaggacgaaggaaatgacgtaagccatgacgtctaatcccacaagaatttccttatataaggaacacaaatcagaaggaagagatcaatcgaaatcaaaatcggaatcgaaatcaaaatcggaatcgaaatctctcatctaagctt

SEQ ID NO: 26 - DNA, 641 bp, artificial sequence
  source: /note=“Description of artificial sequence pMMV double enhanced between EcoR1 and Hind3 sites”
gaattcgtcaacttcgtccacagacatcaacatcttatcgtcctttgaagataagataataatgttgaagataagagtgggagcccccactaaaacattgctttgtcaaaagctaaaaaagatgatgcccgacagccacttgtgtgaagcatgagaagccggtccctccactaagaaaattagtgaagcatcttccagtggtccctccactcacagctcaatcagtgagcaacaggacgaaggaaatgacgtaagccatgacgtctaatcccaacttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgagaagccggtccctccactaagaaaattagtgaagcatcttccagtggtccctccactcacagctcaatcagtgagcaacaggacgaaggaaatgacgtaagccatgacgtctaatcccacaagaatttccttatataaggaacacaaatcagaaggaagagatcaatcgaaatcaaaatcggaatcgaaatcaaaatcggaatcgaaatctctcatctaagctt

SEQ ID NO: 27 - DNA, 348 bp, artificial sequence
  source: /note=“Description of artificial sequence pFMV single enhanced between EcoR1 and Hind3 sites”
gaattcgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagattgcccgggtaatccctctatataagaaggcattcattcccatttgaaggatcatcagatactcaaccaatatttctcactctaagaaattaagagctttgtattcttcaatgagggctaagacccaagctt

SEQ ID NO: 28 - DNA, 562 bp, artificial sequence
  source: /note=“Description of artificial sequence pFMV double enhanced between EcoR1 and Hind3 sites”
gaattcgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagattgcccaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagattgcccgggtaatccctctatataagaaggcattcattcccatttgaaggatcatcagatactcaaccaatatttctcactctaagaaattaagagctttgtattcttcaatgagaggctaagacccaagctt

SEQ ID NO: 29 - DNA, 274 bp, artificial sequence
  source: /note=“Description of artificial sequence pPClSV single enhanced between EcoR1 and Hind3 sites”
gaattcaattcgtcaacgagatcttgagccaatcaaagaggagtgatgttgacctaaagcaataatggagccatgacgtaagggcttacgcccatacgaaataattaaaggctgatgtgacctgtcggtctctcagaacctttactttttatatttggcgtgtatttttaaatttccacggcaatgacgatgtgacctgtgcatccgctttgcctataaataagttttagtttgtattgatcgacacgatcgagaagacacggccataaagctt

SEQ ID NO: 30 - DNA, 452 bp, Artificial sequence
  source: /note=“Description of artificial sequence pPClSV double enhanced between EcoR1 and Hind3 sites”
gaattcgtcaacgagatcttgagccaatcaaagaggagtgatgtagacctaaagcaataatggagccatgacgtaagggcttacgcccatacgaaataattaaaggctgatgtgacctgtcggtctctcagaacctttactttttatgtttggcgtgtatttttaaatttccacggcaatgacgatgtgacccaacgagatcttgagccaatcaaagaggagtgatgtagacctaaagcaataatggagccatgacgtaagggcttacgcccatacgaaataattaaaggctgatgtgacctgtcggtctctcagaacctttactttttatatttggcgtgtatttttaaatttccacggcaatgacgatgtgacctgtgcatccgctttgcctataaataagttttagtttgtattgatcgacacggtcgagaagacacggccataagctt

SEQ ID NO: 31 - protein, 23 aa, artificial sequence
  source: /note=“Description of artificial sequence patatin signal peptide”
MATTKSFLILFFMILATTSSTCA

SEQ ID NO: 32 - DNA, 1356 bp, artificial sequence
  source: /note=“Description of artificial sequence rituximab mature heavy chain (tobacco optimized) sequence”
caagttcaacttcaacaaccaggtgctgaacttgttaagcctggtgcttctgttaagatgtcttgcaaggcttctggatacactttcacatcctacaacatgcattgggttaagcaaactccaggacgtggacttgaatggattggagctatctaccctggaaacggtgatacttcctacaaccagaagttcaagggaaaggctactcttactgctgataagtcctcttccactgcttacatgcaactttcttcactcacttccgaggattctgctgtttattactgcgctaggtccacttattatggtggagattggtacttcaatgtttggggagctggaactactgttactgtgtctgctgcttctactaagggaccatctgtttttccacttgctccatcttctaagtctacttccggtggaactgctgctcttggatgccttgtgaaggattatttcccagagccagtgactgtttcttggaactctggtgctcttacttctggtgttcacactttcccagctgttcttcagtcatctggactttactccctttcttctgttgttactgtgccatcttcttcacttggaactcagacttacatctgcaacgttaaccacaagccatctaacacaaaagtggataagaaggcagagccaaagtcttgtgataagactcatacttgtccaccatgtccagctccagaacttcttggtggtccatctgttttcttgttcccaccaaagccaaaggatactctcatgatctctaggactccagaagttacttgcgttgttgtggatgtttctcatgaggacccagaggttaagttcaactggtacgtggatggtgttgaagttcacaacgctaagactaagccaagataggaacagtacaactctacttaccgtgttgtgtctgtgcttactgttcttcaccaggattggcttaacggaaaagagtacaaatgcaaggtttccaataaggctttgccagctccaattgaaaagactatctccaaggcaaaaggacagcctagagagccacaggtttacactcttccaccatctagagatgagcttactaagaaccaggtttcccttacttgtcttgtgaagggattctacccatctgatattgctgttgagtgggagtcaaacggacagcctgagaacaactacaagactactccaccagtgcttgattctgatggttccttcttcctctactccaaactcactgtggataagtctagatggcagcagggaaatgttttctcttgctccgttatgcatgaggctctccataatcactacactcagaagtccctttctttgtctcctggaaagtga

SEQ ID NO: 33 - protein, 450 aa, artificial sequence
  source: /note=“Description of artificial sequence rituximab mature heavy chain amino acid sequence”
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 69 bp, Artificial sequence
  source: /note=“Description of artificial sequence patatin tobacco non optimized sequence (slightly modified) as in C148 (in front of heavy chain)”
atggccactactaaatcttttttaattttattttttatgatattagcaactactagttcaacatgtgct

SEQ ID NO: 35 - DNA, 642 bp, Artificial sequence
  source: /note=“Description of artificial sequence rituximab mature light chain (tobacco optimized) sequence”
cagattgtgctttctcagtctccagctattctttctgcttccccaggtgaaaaggttacaatgacttgccgtgcttcttcttctgtgtcctacattcattggttccaacagaagccaggatcttctccaaagccatggatctacgctacttctaaccttgcttctggtgttccagttaggttttctggatctggatctggtacttcttactcccttactatttctagagtggaggctgaagatgctgctacttactactgccaacagtggacttctaatccaccaactttcggaggtggaactaagcttgagatcaagaggactgttgctgctccatctgtgtttattttcccaccatctgatgagcaacttaagtctggaactgcttctgttgtgtgccttctcaacaatttctacccaagggaagctaaggttcagtggaaagtggataatgctctccagtctggaaattctcaagagtctgtgactgagcaggattctaaggattccacttactccctttcttctactcttactctctccaaggctgattatgagaagcacaaggtttacgcttgcgaagttactcatcagggactttcttcaccagtgacaaagtccttcaaccgtggagagtgttga

SEQ ID NO: 36 - protein, 213 aa, artificial sequence
  source: /note=“Description of artificial sequence rituximab mature light chain amino aicd sequence”
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 69 bp, artificial sequence
  source: /note=“Description of artificial sequence the patatin tobacco optimized sequence as in C148 (in front of light chain)”
atggccactactaagtccttccttatcctcttcttcatgatccttgctactacttcttctacatgtgct

SEQ ID NO: 38 - DNA, 1491 bp, artificial sequence
  source: /note=“Description of artificial sequence mature GBA (tobacco optimized) sequence”
gctagaccatgcattcctaagtctttcggttactcttctgttgtgtgcgtgtgcaatgctacttactgcgattctttcgatcctcctacttttcctgctcttggtactttttctaggtacgagtctaccaggtctggtagaagaatggaactttctatgggtcctatccaggctaatcatactggtactggtctgcttcttactcttcaacctgagcagaagttccaaaaggttaagggttttggtggtgctatgactgatgctgctgctcttaatattctggctctttctcctcctgctcaaaacttgctgctgaagtcttacttcagcgaagagggtatcggttacaacattattagggtgccaatggcttcctgcgatttctctattaggacttatacctacgctgatacccctgatgatttccagcttcacaactttagcctgcctgaagaggataccaagctgaagattcctcttattcatagggctctgcagcttgctcaaagacctgtttctcttttggcttctccttggacttctcctacttggcttaagactaatggtgctgtgaacggtaagggttctcttaagggtcaacctggtgatatctaccatcaaacttgggctagatacttcgtgaagttccttgatgcttacgctgagcataagttgcagttttgggctgttactgctgagaatgagccttctgctggtcttttgtctggttatccttttcagtgccttggtttcactcctgaacatcagagggatttcattgctagagatttgggtcctacccttgctaattctactcatcataacgtgaggctgctgatgcttgatgatcagagacttcttttgcctcactgggctaaggttgtgcttactgatcctgaagctgctaagtacgttcacggtattgctgttcactggtacttggattttctggctcctgctaaggctactcttggtgaaactcataggcttttccctaacaccatgctttttgcttcagaggcttgcgttggttctaagttttgggaacagtctgtgagacttggatcttgggatagaggtatgcagtacagccactctattattaccaacctgctgtaccatgtggtgggttggactgattggaatcttgctcttaatcctgagggtggtcctaattgggttaggaatttcgtggatagccctatcatcgtggatattaccaaggataccttctacaagcagcctatgttctaccatcttggtcacttcagcaagttcattccagaaggttctcagagggttggacttgttgcttctcaaaagaacgatcttgatgctgtggctcttatgcaccctgatggttctgctgttgttgttgtgcttaacaggtctagcaaggatgtgcctctgactatcaaagatcctgctgttggtttcttagagaccatttctcctggttactctattcacacctacctttggcgtcgacaa

SEQ ID NO: 39 - protein, 497 aa, artificial sequence
  source: /note=“Description of artificial sequence mature GBA amino acid sequence”
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 40 - DNA, 69 bp, artificial sequence
  source: /note=“Description of artificial sequence patatin tobacco optimized sequence”
atggctactactaagtctttcctgatcctgttcttcatgattcttgctactacctcgagcacgtgtgct